(19) United States
(12) Patent Application Publication
(10) Pub. No.: US 2024/0018228 A1
(43) Pub. Date: Jan. 18, 2024

(54) ANTIBODIES SPECIFIC TO CCL21 AND METHODS OF USE

(71) Applicants: BOARD OF REGENTS, THE UNIVERSITY OF TEXAS SYSTEM, Austin, TX (US); THE UNIVERSITY OF FLORIDA RESEARCH FOUNDATION, INC., Graineseville, FL (US)

(72) Inventor: Robert Hromas, San Antonio, TX (US)

(21) Appl. No.: 18/043,570

(22) PCT Filed: Aug. 27, 2021
(86) PCT No.: PCT/US2021/047897

§ 371 Date: Feb. 28, 2023

Related U.S. Application Data
(60) Provisional application No. 63/071,549, filed on Aug. 28, 2020.
(60) Provisional application No. 63/217,534, filed on Jul. 1, 2021.

(51) Int. Cl. C07K 16/24; A61P 1/00

(52) U.S. Cl. CPC C07K 16/24; A61P 1/00; C07K 2317/24; C07K 2317/34; C07K 2317/565; C07K 2317/76; C07K 2317/92

(57) ABSTRACT

Disclosed herein are anti-CCL21 antibodies useful in treating autoimmune diseases, treating or preventing allograft transplant rejection, and treating metastatic cancer or preventing cancer metastasis in a subject.

### CROSS REFERENCE TO RELATED APPLICATIONS

This application claims the benefit of the filing dates of U.S. Provisional Application No. 63/071,549, filed on Aug. 28, 2020; and U.S. Provisional Application No. 63/217,534, filed on Jul. 1, 2021. The content of these earlier filed applications is hereby incorporated by reference in their entirety.

### REFERENCE TO A SEQUENCE LISTING

The Sequence Listing submitted herein as a text file named “21105_0079P1_SL.txt,” created on Aug. 26, 2021, and having a size of 24,576 bytes is hereby incorporated by reference pursuant to 37 C.F.R. § 1.52(e)(5).

### BACKGROUND

As of 2012, the National Institutes of Health estimated that 24 million individuals in the United States alone have some form of autoimmune disease, with an estimated global prevalence of 5-10%. The American Autoimmune Related Diseases Association predicted that in the U.S. the top 7 diseases cost ˜$51-$70 billion annually. T cells mediate a large number of these autoimmune inflammatory diseases including type I diabetes (T1D), rheumatoid arthritis (RA), psoriasis (psoriatic arthritis), Multiple Sclerosis (MS), and inflammatory bowel disease (IBD). In these diseases, T cells escape central tolerance mechanisms and are sensitized by self-antigens (autoantigen). Thus, in these patients, their own body's immune system begins to attack normal healthy tissues, (T1D, pancreatic islets; RA, connective tissue of joints; Psoriasis, skin; MS, myelin of the brain; IBD, digestive tract) and cause autoinflammation, which over time leads to the destruction of the target tissue.

### SUMMARY

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1; a complementarity determining region light chain 2 (CDRL2) amino acid sequence of SEQ ID NO: 2; and a complementarity determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 68; a complementarity determining region light chain 2 (CDRL2) amino acid sequence of SEQ ID NO: 2; and a complementarity determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10.

Disclosed herein are isolated antibodies comprising a light chain variable region amino acid sequence of SEQ ID NO: 7 and a heavy chain variable region amino acid sequence of SEQ ID NO: 14.

Disclosed herein are isolated antibodies comprising a light chain variable region amino acid sequence of SEQ ID NO: 64 and a heavy chain variable region amino acid sequence of SEQ ID NO: 60.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 68; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 7 and a heavy chain variable region amino acid sequence of SEQ ID NO: 14, wherein the isolated antibody comprises 1, 2, 3, 4, or 5 conservative amino acid substitutions in the light or heavy chain variable region amino acid sequences.

Disclosed herein are isolated antibodies comprising a light chain variable region amino acid sequence of SEQ ID NO: 64 and a heavy chain variable region amino acid sequence of SEQ ID NO: 60, wherein the isolated antibody comprises 1, 2, 3, 4, or 5 conservative amino acid substitutions in the light or heavy chain variable region amino acid sequences.

Disclosed herein are isolated antibody variants comprising a complementarity determining region light chain 1 (CDRL1) comprising the amino acid sequence of SEQ ID NOs: 38, 39, 40, 41, 42 or 43; a complementarity determining region light chain 2 (CDRL2) comprising the amino acid sequence of SEQ ID NOs: 44, 45, 46, 47, 48, 49, 50, or 51; and a complementarity determining region light chain 3 (CDRL3) comprising the amino acid sequence of SEQ ID NOs: 52, 53, 54 or 55; and a complementarity determining region heavy chain 1 (CDRH1) comprising the amino acid sequence of SEQ ID NOs: 16, 17, 18, 19, 20 or 21; a complementarity determining region heavy chain 2 (CDRH2) comprising the amino acid sequence of SEQ ID NOs: 22, 23, 24, 25, 26, 27, 28, 29, or 30; and a complementarity determining region heavy chain 3 (CDRH3) comprising the amino acid sequence of SEQ ID NOs: 31, 32, 33, 34, 35, 36 or 37.

Disclosed herein are isolated antibodies comprising a light chain variable region and a heavy chain variable region, wherein the light chain variable region comprises: a) a variant complementarity determining region light chain 1 (CDRL1) comprising positions 24-34 of SEQ ID NO: 7, wherein the variant CDRL1 comprises one or two amino acid substitutions; b) a variant complementarity determining region light chain 2 (CDRL2) comprising positions of SEQ ID NO: 7, wherein the variant CDRL2 comprises one or two amino acid substitutions; and b) a variant complementarity determining region light chain 3 (CDRL3) comprising positions 89-97 of SEQ ID NO: 7, wherein the variant CDRL3 comprises one or two amino acid substitutions; wherein the heavy chain variable region comprises: d) a variant complementarity determining region heavy chain 1 (CDRH1) comprising positions 31-35 of SEQ ID NO: 14, wherein the variant CDRH1 comprises one or two amino acid substitutions; e) a variant complementarity determining region heavy chain 2 (CDRH2) comprising positions 50-66 of SEQ ID NO: 14, wherein the variant CDRH2 comprises one or two amino acid substitutions; and 0 a variant complementarity determining region heavy chain 3 (CDRH3) comprising positions 99-103 of SEQ ID NO: 14, wherein the variant CDRH3 comprises one or two amino acid substitutions.

Disclosed herein are isolated antibodies comprising a light chain variable region and a heavy chain variable region, wherein the light chain variable region comprises: a) a variant complementarity determining region light chain 1 (CDRL1) comprising positions 24-34 of SEQ ID NO: 64, wherein the variant CDRL1 comprises one or two amino acid substitutions when compared to positions 24-34 of SEQ ID NO: 64; b) a variant complementarity determining region light chain 2 (CDRL2) comprising positions 50-56 of SEQ ID NO: 64, wherein the variant CDRL2 comprises one or two amino acid substitutions when compared to positions 50-56 of SEQ ID NO: 64; and b) a variant complementarity determining region light chain 3 (CDRL3) comprising positions 89-97 of SEQ ID NO: 64, wherein the variant CDRL3 comprises one or two amino acid substitutions when compared to positions 89-97 of SEQ ID NO: 64; wherein the heavy chain variable region comprises: d) a variant complementarity determining region heavy chain 1 (CDRH1) comprising positions 31-35 of SEQ ID NO: 60, wherein the variant CDRH1 comprises one or two amino acid substitutions when compared to positions 31-35 of SEQ ID NO: 60; e) a variant complementarity determining region heavy chain 2 (CDRH2) comprising positions 50-66 of SEQ ID NO: 60, wherein the variant CDRH2 comprises one or two amino acid substitutions when compared to positions 50-66 of SEQ ID NO: 60; and 0 a variant complementarity determining region heavy chain 3 (CDRH3) comprising positions 99-103 of SEQ ID NO: 60, wherein the variant CDRH3 comprises one or two amino acid substitutions when compared to positions 99-103 of SEQ ID NO: 60.

### BRIEF DESCRIPTION OF THE DRAWINGS

FIGS. 1A-D show the results of screening anti-CCL21 monoclonal antibody clones against the amino terminus of CCL21. FIG. 1A shows screening of anti-CCL21 monoclonal antibody clones by western blotting for binding to the human CCL21 protein as a single band. FIG. 1B shows the cross reactivity of anti-CCL21 monoclonal antibody clones to human and mouse CCL21 protein. The clones were then screened for lack of binding to murine CCL21 by slot blot hybridization. FIG. 1C shows that the screening of a peptide from the CCR7-interacting amino terminal region of CCL21 could compete off the monoclonal from binding CCL21 using slot blot hybridization. FIG. 1D shows screening of monoclonal antibody clones for lack of binding to the related human chemokine CCL19. Clone 8 results are shown. Pep 1 is SEQ ID NO: 56. Pep 2 is SEQ ID NO: 57.

FIG. 2 shows the results of screening of C8 for immunohistologic recognition of CCL21 in a tonsilar lymphoid biopsy specimen. C8 was positive for binding to appropriate CCL21-expressing cells in human tonsil lymph node, (left panels). C8 did not recognize synovium from rheumatoid arthritis serves as a negative control (right panels).

FIGS. 3A-B shows the screening of monoclonal antibody clones against CCL21-mediated T-cell chemotaxis. FIG. 3A shows inhibition of migration in transwell chemotaxis assays of human helper T-cells (CD3⁺/CD4⁺) towards human CCL21 by the 33 murine monoclonal antibody clones that passed the screening process outlined in FIG. 1. FIG. 3B shows that C8 also blocked migration of three distinct normal donor human Th-cells towards CCL21, indicating this is a general phenomenon. For the migration assays, data are the mean±SD of triplicate wells, performed twice. Student T tests were performed for statistical analysis for this figure and FIG. 3. * p<0.05, ** p<0.01, *** p<0.001 and **** p<0.0001 when compared to control.

FIGS. 4A-F show the results of screening for inhibition of T-cell (Th) migration towards CCL21 by C8. FIG. 4A shows the flow cytometric gating strategy for isolation of human Th-cell subpopulations for testing the effects of C8 on migration towards CCL21. Migration of defined populations of T cell subsets was determined using flow cytometry of the lower versus upper chambers in transwell chemotaxis assays with background migration (cells that migrated toward media with no chemokine) subtracted from total cells. FIG. 4B shows surface biomarker identification of Th-cell subsets tested in these experiments. FIGS. 4C-F show the fractional migration towards CCL21 inhibited by C8 in these T-cell subsets. Naïve Th-cells had the greatest migration towards CCL21 and were the most inhibited. Effect of C8 on migration towards CCL21 decreases as Th-cells become more mature. Data are the mean±SD of triplicate wells, performed twice. Student T tests were performed for statistical analysis.

FIGS. 5A-H shows the immunohistology of venule endothelial expression of CCL21 in intestinal mucosal autoimmune diseases. FIG. 5A shows 40× magnification of 1 of 3 Crohn's disease intestinal mucosa biopsy immunohistologically stained with C8 (anti-CCL21 monoclonal antibody). Two of 3 had similar positive results as shown here. FIG. 5B shows 100× magnification of (FIG. 5A). FIG. 5C shows 40× magnification of 1 of 3 ulcerative colitis colonic mucosa biopsy immunohistologically stained with C8. FIG. 5D shows 100× magnification of (FIG. 5C). FIG. 5E shows 40× magnification of 1 of 6 colonic mucosal biopsies of celiac sprue disease immunohistologically stained with C8. FIG. 5F shows 100× magnification of (FIG. 5E). FIG. 5G shows that normal duodenum does not express CCL21. One of 3 biopsy samples shown here that is representative of 3. FIG. 5H shows that normal colon does not express CCL21. One representative sample of 3 biopsies shown here.

FIGS. 6A-D shows the screening of fully humanized anti-CCL21 monoclonal antibody clones derived from clone C8. FIG. 6A shows the inhibition of migration in transwell chemotaxis assays of helper T cells (CD3⁺/CD4⁺) towards 1200 ng/ml rhCCL21 by the 16 humanized clones, referred to as V1 to V16, used at 100 μg/ml. The versions of humanized clones tested inhibited T cell migration towards CCL21 to some degree. However, V6 was the most potent at inhibiting T cell migration towards CCL21 (arrow). Data are the mean±SD of triplicate wells. One-way ANOVA with post-hoc Tukey's multiple comparison tests were performed for statistical analysis. The groups had a p<0.0001 when compared to control. FIGS. 6B-D confirm V6 inhibition of CD3⁺/CD4⁺/CD8⁻/CCR7⁺/CD45RA⁺/CD27⁺ naïve Th-cell chemotaxis towards 100, 500, 800, 1200, and 1600 ng/ml rhCCL21 using CD3⁺ peripheral blood naïve Th-cells from 3 normal human donors. Data are the mean±SD of triplicate wells.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list. Further, the term “or” means “and/or” unless explicitly indicated to refer to alternatives only or the alternatives are mutually exclusive, although the disclosure supports a definition that refers to only alternatives and “and/or.”

As used herein, the term “another” means at least a second or more.

As used herein, the term “about” is used to indicate that a value includes the inherent variation of error for the device, the method being employed to determine the value, or the variation that exists among the study subjects.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In some aspects, a subject is a human. The term does not denote a particular age or sex. Thus, adult, child, adolescent and newborn subjects, as well as fetuses, whether male or female, are intended to be covered. As used herein, the term “subject” refers to either a human or a non-human animal, such as primates, mammals, and vertebrates having an autoimmune disease or organ transplant or diagnosed with an autoimmune disease. In some aspects, the subject is a transplant recipient or transplant patient. In some aspects, the subject in need will or is predicted to benefit from anti-CCL21 antibody treatment.

As used herein, the term “patient” refers to a subject afflicted with a disease or disorder or is a transplant or organ recipient. The term “patient” includes human and veterinary subjects. In some aspects of the disclosed methods, the “patient” has been diagnosed with a need for treatment for an autoimmune disease or to prevent graft rejection, such as, for example, prior to the administering step.

As used herein, the term “treat,” “treatment,” or “treating” refers to administration or application of a therapeutic agent to a subject in need thereof, or performance of a procedure or modality on a subject, for the purpose of obtaining at least one positive therapeutic effect or benefit, such as treating a disease or health-related condition. For example, a treatment can include administration of a pharmaceutically effective amount of an antibody, or a composition or formulation thereof that specifically binds to CCL21 for the purpose of treating various autoimmune diseases or preventing graft rejection. The terms “treatment regimen,” “dosing regimen,” or “dosing protocol,” are used interchangeably and refer to the timing and dose of a therapeutic agent, such as an anti-CCL21 antibody as described herein.

As used herein, the term “therapeutic benefit” or “therapeutically effective” refers the promotion or enhancement of the well-being of a subject in need (e.g., a subject with an autoimmune disease or diagnosed with an autoimmune disease or cancer or a subject undergoing an organ transplant) with respect to the medical treatment, therapy, dosage administration, of a condition, particularly as a result of the use of the anti-CCL21 antibodies and the performance of the described methods. This includes, but is not limited to, a reduction in the frequency or severity of the signs or symptoms of a disease. In some aspects, treatment of cancer or metastatic cancer may involve, for instance, a reduction in the size of a tumor, a reduction in the invasiveness or severity of a tumor, a reduction infiltration of cancer cells into a peripheral tissue or organ; a reduction in the growth rate of the tumor or cancer, or the prevention or reduction of metastasis. Treatment of cancer may also refer to achieving a sustained response in a subject or prolonging the survival of a subject with cancer.

As used herein, the term “administer” or “administration” refers to the act of physically delivering, e.g., via injection or an oral route, a substance as it exists outside the body into a patient, such as by oral, subcutaneous, mucosal, intradermal, intravenous, intramuscular delivery and/or any other method of physical delivery described herein or known in the art. When a disease, disorder or condition, or a symptom thereof, is being treated therapeutically, administration of the substance typically occurs after the onset of the disease, disorder or condition or symptoms thereof. Prophylactic treatment involves the administration of the substance at a time prior to the onset of the disease, disorder or condition or symptoms thereof.

As used herein, the term “effective amount” refers to the quantity or amount of a therapeutic (e.g., an antibody or pharmaceutical composition provided herein) which is sufficient to reduce, diminish, alleviate, and/or ameliorate the severity and/or duration of a cancer or a symptom related thereto. This term also encompasses an amount necessary for the reduction or amelioration of the advancement or progression of a cancer or an autoimmune disease; the reduction or amelioration of the recurrence, development of a cancer or onset of an autoimmune disease or disorder or graft rejection; and/or the improvement or enhancement of the prophylactic or therapeutic effect(s) of another cancer therapy. In some aspects, the effective amount of an antibody provided herein is from about or equal to 0.1 mg/kg (mg of antibody per kg weight of the subject) to about or equal to 100 mg/kg. In some aspects, an effective amount of an antibody provided therein is about or equal to 0.1 mg/kg, about or equal to 0.5 mg/kg, about or equal to 1 mg/kg, about or equal to 3 mg/kg, about or equal to 5 mg/kg, about or equal to 10 mg/kg, about or equal to 15 mg/kg, about or equal to 20 mg/kg, about or equal to 25 mg/kg, about or equal to 30 mg/kg, about or equal to 35 mg/kg, about or equal to 40 mg/kg, about or equal to 45 mg/kg, about or equal to mg/kg, about or equal to 60 mg/kg, about or equal to 70 mg/kg, 80 mg/kg, 90 mg/kg, or 100 mg/kg. These amounts are meant to include amounts and ranges therein. In some aspects, “effective amount” also refers to the amount of an antibody provided herein to achieve a specified result (e.g., preventing, blocking, or inhibiting cell surface CCL21 binding to cell surface CCR7; or preventing, blocking, or inhibiting CCL21/CCR7 mediated immune system over activity).

The term “in combination” in the context of the administration of other therapies (e.g., other agents, cancer drugs, cancer therapies, immunosuppressants) includes the use of more than one therapy (e.g., drug therapy and/or cancer therapy and/or immunosuppressants). Administration “in combination with” one or more further therapeutic agents includes simultaneous (e.g., concurrent) and consecutive administration in any order. The use of the term “in combination” does not restrict the order in which therapies are administered to a subject. By way of nonlimiting example, a first therapy (e.g., agent, such as an anti-CCL21 antibody) may be administered before (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with an autoimmune disease or disorder or an organ transplant.

In some aspects, the second therapy or agent that can be used in combination with drugs used to treat autoimmune disorders, transplant graft rejection or graft versus host diseases include but are not limited to tofacitinib, baricitinib, secukinumab, cyclosporin, tacrolimus, rapamycin, everolimus, mycophenolate, azathioprine, leflunomide, abatacept, adalimumab, anakinra, certolizumab, etanercept, golimumab, infliximab, ixekizumab, natalizumab, rituximab, tocilizumab, ustekinumab, vedolizumab, basiliximab, daclizumab, ibrutinib, acalabrutinib, ruxolitinib, and fedratinib.

In some aspects, the combination of therapies (e.g., use of agents, including therapeutic agents) may be more effective than the additive effects of any two or more single therapy (e.g., have a synergistic effect). For example, a synergistic effect of a combination of therapeutic agents frequently permits the use of lower dosages of one or more of the agents and/or less frequent administration of the agents to a cancer patient. The ability to utilize lower dosages of therapeutics and cancer therapies and/or to administer the therapies less frequently reduces the potential for toxicity associated with the administration of the therapies to a subject without reducing the effectiveness of the therapies. In addition, a synergistic effect may result in improved efficacy of therapies in the treatment or alleviation of a cancer, an autoimmune disease or graft rejection. Also, a synergistic effect demonstrated by a combination of therapies (e.g., therapeutic agents) may avoid or reduce adverse or unwanted side effects associated with the use of any single therapy.

As used herein, the term “comprising” can include the aspects “consisting of” and “consisting essentially of” “Comprising” can also mean “including but not limited to.”

“Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity. For example, determining the amount of a disclosed polypeptide, protein, gene or antibody in a sample as used herein can refer to the steps that the skilled person would take to measure or ascertain some quantifiable value of the polypeptide protein, gene or antibody in the sample. The art is familiar with the ways to measure an amount of the disclosed polypeptide, proteins, genes or antibodies in a sample.

As used herein, the terms “disease” or “disorder” or “condition” are used interchangeably referring to any alternation in state of the body or of some of the organs, interrupting or disturbing the performance of the functions and/or causing symptoms such as discomfort, dysfunction, distress, or even death to the person afflicted or those in contact with a person. A disease or disorder or condition can also related to a distemper, ailing, ailment, disorder, sickness, illness, complaint, affection. In some aspects, the disease or disorder or condition can be a cancer, metastatic cancer, an autoimmune disease or disorder, or an organ transplant. In some aspects, the autoimmune disease or disorder can be an inflammatory bowel disease such as Crohn's disease or ulcerative colitis, type 1 diabetes, psoriasis, multiple sclerosis, Systemic lupus erythromatosis, scleroderma, autoimmune diseases of the thyroid, such as Grave's or Hashimoto's disease, Sjogren's syndrome, celiac disease, autoimmune vasculitis including temporal arteritis, Addison's disease, demyelinating polyneuropathies, polymyalgia rheumatica, or ankylosing spondylitis. In some aspects, the cancer can be breast cancer, colon cancer, lymphatic system cancers, pancreatic cancer, lung cancer, skin cancer (e.g., melanoma), esophageal cancer, head and neck cancers, and stomach cancer.

As used herein, the term “chemokine (C—C motif) ligand 21” or “CCL21” refers to a polypeptide (the terms “polypeptide” and “protein” are used interchangeably herein) or any native CCL21 from any vertebrate source, including mammals such as primates (e.g., humans, cynomolgus monkey (cyno)), dogs, and rodents (e.g., mice and rats), unless otherwise indicated, and, in certain aspects, included various CCL21 isoforms, related CCL21 polypeptides, including SNP variants thereof. An exemplary amino acid sequence of human CCL21 is accession number NP_002980.1.

Abbreviations for the amino acid residues that comprise polypeptides and peptides described herein, and conservative substitutions for these amino acid residues are shown in Table 1 below. A polypeptide that contains one or more conservative amino acid substitutions or a conservatively modified variant of a polypeptide described herein refers to a polypeptide in which the original or naturally occurring amino acids are substituted with other amino acids having similar characteristics, for example, similar charge, hydrophobicity/hydrophilicity, side-chain size, backbone conformation, structure and rigidity, etc. Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity.

TABLE 1
Amino Acid Residues and Examples of
Conservative Amino Acid Substitutions

| Original residue | |
|---|---|
| Three letter code and | Conservative |
| Single letter code | substitution(s) |
| Alanine (Ala) (A) | Gly; Ser |
| Arginine (Arg) (R) | Lys; His |
| Asparagine (Asn) (N) | Gln; His |
| Aspartic Acid (Asp) (D) | Glu; Asn |
| Cysteine (Cys) (C) | Ser; Ala |
| Glutamine (Gln) (Q) | Asn |
| Glutamic Acid (Glu) (E) | Asp; Gln |
| Glycine (Gly) (G) | Ala |
| Histidine (His) (H) | Asn; Gln |
| Isoleucine (Ile) (I) | Leu; Val |
| Leucine (Leu) (L) | Ile; Val |
| Lysine (Lys) (K) | Arg; His |
| Methionine (Met) (M) | Leu; Ile; Tyr |
| Phenylalanine (Phe) (F) | Tyr; Met; Leu |
| Proline (Pro) (P) | Ala |
| Serine (Ser) (S) | Thr |
| Threonine (Thr) (T) | Ser |
| Tryptophan (Trp) (W) | Tyr; Phe |
| Tyrosine (Tyr) (Y) | Trp; Phe |
| Valine (Val) (V) | Ile; Leu |

As used herein, the term “polypeptide” or “peptide” refers to a polymer of amino acids of three or more amino acids in a serial array, linked through peptide bonds. As used herein, the term “amino acid sequence” refers to a list of abbreviations, letters, characters or words representing amino acid residues. “Polypeptides” can be proteins, protein fragments, protein analogs, oligopeptides and the like. The amino acids that comprise the polypeptide may be naturally derived or synthetic. The polypeptide may be purified from a biological sample. For example, a CCL21 polypeptide or peptide may be composed of at least 3, 4, 5, 6, 7, 8, 9, 10, 11, 12, 13, 14, 15, 16, 17, 18, 19, 20, 21, 22, 23, 24, or 25 contiguous amino acids of human CCL21. In some aspects, the polypeptide has at least 25, 30, 35, 40, 45, 50, 60, 65, 70, 75, 80, 85, 90, 95, 100, 105, 110, 115, 120, 125, 130, or 135, contiguous amino acids of human CCL21. In some aspects, the CCL21 polypeptide comprises at least contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least contiguous 100 amino acid residues, at least 125 contiguous amino acid residues, at least 134 contiguous amino acid residues of the amino acid sequence of the CCL21 polypeptide.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

As used herein, the term “analog” refers to a polypeptide that possesses a similar or identical function as a reference polypeptide but does not necessarily comprise a similar or identical amino acid sequence of the reference polypeptide, or possess a similar or identical structure of the reference polypeptide. The reference polypeptide may be a CCL21 polypeptide, a fragment of a CCL21 polypeptide, or an anti-CCL21 antibody. A polypeptide that has a similar amino acid sequence with a reference polypeptide refers to a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the reference polypeptide, which can be a CCL21 polypeptide or an anti-CCL21 antibody as described herein. A polypeptide with similar structure to a reference polypeptide refers to a polypeptide that has a secondary, tertiary, or quaternary structure similar to that of the reference polypeptide, which can be a CCL21 polypeptide or an anti-CCL21 antibody described herein. The structure of a polypeptide can determined by methods known to those skilled in the art, including, but not limited to, X-ray crystallography, nuclear magnetic resonance (NMR), and crystallographic electron microscopy.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

As used herein, the term “variant” when used in relation to a CCL21 polypeptide or to an anti-CCL21 antibody refers to a polypeptide or an anti-CCL21 antibody having one or more amino acid sequence substitutions, deletions, and/or additions as compared to a native or unmodified CCL21 sequence or anti-CCL21 antibody sequence. For example, a CCL21 polypeptide or to an anti-CCL21 antibody refers to a polypeptide or an anti-CCL21 antibody having one or more amino acid sequence substitutions, deletions, and/or additions as compared to a native or unmodified CCL21 sequence or anti-CCL21 antibody sequence can have about 1 to about 25, about 1 to about 20, about 1 to about 15, about 1 to about 10, or about 1 to about 5 amino acid sequence substitutions, deletions, and/or additions as compared to a native or unmodified CCL21 sequence or anti-CCL21 antibody sequence. A CCL21 variant can result from one or more (such as, for example, about 1 to about 25, about 1 to about 20, about 1 to about 15, about 1 to about 10, or about 1 to about 5) changes to an amino acid sequence of a native CCL21. Also by way of example, a variant of an anti-CCL21 antibody can result from one or more (such as, for example, about 1 to about 25, about 1 to about 20, about 1 to about 15, about 1 to about 10, or about 1 to about 5 changes to an amino acid sequence of a native or previously unmodified anti-CCL21 antibody. Variants can be naturally occurring, such as allelic or splice variants, or can be artificially constructed. Polypeptide variants can be prepared from the corresponding nucleic acid molecules encoding the variants.

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions can include those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, a variant may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH2(CH2)nCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

A “conservative substitution” with reference to amino acid sequence refers to replacing an amino acid residue with a different amino acid residue having a side chain with similar physiochemical properties. For example, conservative substitutions can be made among amino acid residues with hydrophobic side chains (e.g., Met, Ala, Val, Leu, and Ile), among residues with neutral hydrophilic side chains (e.g., Cys, Ser, Thr, Asn and Gln), among residues with acidic side chains (e.g., Asp, Glu), among amino acids with basic side chains (e.g., His, Lys, and Arg), or among residues with aromatic side chains (e.g., Trp, Tyr, and Phe). As known in the art, conservative substitution usually does not cause significant change in the protein conformational structure, and therefore could retain the biological activity of a protein.

The term “identity” refers to a relationship between the sequences of two or more polypeptide molecules or two or more nucleic acid molecules, as determined by aligning and comparing the sequences. “Percent identity” means the percent of identical residues between the amino acids or nucleotides in the compared molecules and is calculated based on the size of the smallest of the molecules being compared. For these calculations, gaps in alignments (if any) must be addressed by a particular mathematical model or computer program (e.g., an “algorithm”). Methods that may be used to calculate the identity of the aligned nucleic acids or polypeptides include those described in Lesk, A. M., ed., 1988, Computational Molecular Biology, New York: Oxford University Press; Smith, D. W., ed., 1993, Biocomputing Informatics and Genome Projects, New York: Academic Press; Griffin, A. M., et al., 1994, Computer Analysis of Sequence Data, Part I, New Jersey: Humana Press; von Heinje, G., 1987, Sequence Analysis in Molecular Biology, New York: Academic Press; Gribskov, M. et al., 1991, Sequence Analysis Primer, New York: M. Stockton Press; and Carillo et al., 1988, Applied Math., 48:1073.

In calculating percent identity, the sequences being compared can be aligned in a way that gives the largest match between the sequences. An example of a computer program that can be used to determine percent identity is the GCG program package, which includes GAP (Devereux et al., 1984, Nucl. Acid Res., 12:387; Genetics Computer Group, University of Wisconsin, Madison, WI), which is a computer algorithm used to align the two polypeptides or polynucleotides to determine their percent sequence identity. The sequences can be aligned for optimal matching of their respective amino acid or nucleotide sequences (the “matched span” as determined by the algorithm). A gap opening penalty (which is calculated as 3 times the average diagonal, wherein the “average diagonal” is the average of the diagonal of the comparison matrix being used, and the “diagonal” is the score or number assigned to each perfect amino acid match by the particular comparison matrix; and a gap extension penalty (which is usually 1/10 times the gap opening penalty), as well as a comparison matrix such as PAM 250 or BLOSUM 62, are used in conjunction with the algorithm. In some aspects, a standard comparison matrix (see, Dayhoff et al., 1978, Atlas of Protein Sequence and Structure 5:345-352 for the PAM 250 comparison matrix; Henikoff et al., 1992, Proc. Natl. Acad. Sci. USA 89:10915-10919 for the BLOSUM 62 comparison matrix) is also used by the algorithm. Exemplary parameters for determining percent identity for polypeptides or nucleotide sequences using the GAP program include the following: (i) Algorithm: Needleman et al., 1970, J. Mol. Biol., 48:443-453; (ii) Comparison matrix: BLOSUM 62 from Henikoff et al., Id.; (iii) Gap Penalty: 12 (but with no penalty for end gaps); (iv) Gap Length Penalty: 4; and (v) Threshold of Similarity: 0.

Certain alignment schemes for aligning two amino acid sequences can result in matching only a short region of the two sequences, and this small aligned region can have very high sequence identity even though there is no significant relationship between the two full-length sequences. Accordingly, the selected alignment method (e.g., the GAP program) can be adjusted if so desired to result in an alignment that spans a representative number of amino acids, for example, at least 50 contiguous amino acids, of the target polypeptide.

Percent (%) amino acid sequence identity with respect to a reference polypeptide sequence is defined as the percentage of amino acid residues in a candidate sequence that is identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill of the practitioner in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

As used herein, the term “derivative” refers to a polypeptide that comprises an amino acid sequence of a reference polypeptide that has been altered by the introduction of amino acid residue substitutions, deletions or additions. The reference polypeptide can be a CCL21 polypeptide or an anti-CCL21 antibody. The term “derivative” as used herein also refers to a CCL21 polypeptide or an anti-CCL21 antibody that has been chemically modified, e.g., by the covalent attachment of any type of molecule to the polypeptide. For example, a CCL21 polypeptide or an anti-CCL21 antibody can be chemically modified, e.g., by glycosylation, acetylation, pegylation, phosphorylation, amidation, derivatization by known protecting/blocking groups, proteolytic cleavage, linkage to a cellular ligand, linkage to a peptide or protein tag molecule, or other protein, etc. The derivatives are modified in a manner that is different from the naturally occurring or starting peptide or polypeptides, either in the type or location of the molecules attached. Derivatives may further include deletion of one or more chemical groups which are naturally present on the peptide or polypeptide. A derivative of a CCL21 polypeptide or an anti-CCL21 antibody may be chemically modified by chemical modifications using techniques known to those of skill in the art, including, but not limited to, specific chemical cleavage, acetylation, formulation, metabolic synthesis by tunicamycin, etc. Further, a derivative of a CCL21 polypeptide or an anti-CCL21 antibody can contain one or more non-classical amino acids. A polypeptide derivative possesses a similar or identical function as the reference polypeptide, which can be a CCL21 polypeptide or an anti-CCL21 antibody described herein, especially an anti-CCL21 monoclonal antibody.

The term “fusion protein” as used herein refers to a polypeptide that includes amino acid sequences of at least two heterologous polypeptides. The term “fusion” when used in relation to a CCL21 polypeptide or to an anti-CCL21 antibody refers to the joining, fusing, or coupling of a CCL21 polypeptide or an anti-CCL21 antibody, variant and/or derivative thereof, with a heterologous peptide or polypeptide. In some aspects, the fusion protein retains the biological activity of the CCL21 polypeptide or the anti-CCL21 antibody. In some aspects, the fusion protein includes a CCL21 antibody VH region, VL region, VH CDR (one, two or three VH CDRs), and/or VL CDR (one, two or three VL CDRs) coupled, fused, or joined to a heterologous peptide or polypeptide, wherein the fusion protein binds to an epitope on a CCL21 protein or peptide. Fusion proteins may be prepared via chemical coupling reactions as practiced in the art, or via molecular recombinant technology.

As used herein, the term “composition” refers to a product containing specified component ingredients (e.g., a polypeptide or an antibody provided herein) in, optionally, specified or effective amounts, as well as any desired product which results, directly or indirectly, from the combination or interaction of the specific component ingredients in, optionally, the specified or effective amounts.

As used herein, the term “carrier” includes pharmaceutically acceptable carriers, excipients, diluents, vehicles, or stabilizers that are nontoxic to the cell or mammal being exposed thereto at the dosages and concentrations employed. Often, the physiologically acceptable carrier is an aqueous pH buffered solution. Examples of physiologically acceptable carriers include buffers such as phosphate, citrate, succinate, and other organic acids; antioxidants including ascorbic acid; low molecular weight (e.g., less than about 10 amino acid residues) polypeptides; proteins, such as serum albumin, gelatin, or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose, sucrose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™. The term “carrier” can also refer to a diluent, adjuvant (e.g., Freund's adjuvant, complete or incomplete), excipient, or vehicle with which the therapeutic is administered. Such carriers, including pharmaceutical carriers, can be sterile liquids, such as water and oils, including those of petroleum, animal, vegetable or synthetic origin, such as peanut oil, soybean oil, mineral oil, sesame oil and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include, without limitation, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences, (1990) Mack Publishing Co., Easton, PA. Compositions, including pharmaceutical compounds, can contain a therapeutically effective amount of an anti-CCL21 antibody in isolated or purified form, together with a suitable amount of carrier so as to provide the form for proper administration to the subject (e.g., patient). The composition or formulation should suit the mode of administration.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, PA, which is hereby incorporated by reference in its entirety.

As used herein, the term “pharmaceutically acceptable” or “pharmacologically acceptable” refers to molecular entities, formulations and compositions that do not produce an adverse, allergic, or other untoward or unwanted reaction when administered, as appropriate, to an animal, such as a human. The preparation of a pharmaceutical composition comprising an antibody or additional active ingredient are known to those of skill in the art in light of the present disclosure, as exemplified by Remington's Pharmaceutical Sciences, Id. Moreover, for animal (e.g., human) administration, it will be understood that preparations should meet sterility, pyrogenicity, general safety, and purity standards as required by a regulatory agency of the Federal or a state government, such as the FDA Office of Biological Standards or as listed in the U.S. Pharmacopeia, European Pharmacopeia, or other generally recognized Pharmacopeia for use in animals, and more particularly, in humans.

The term “pharmaceutical formulation” refers to a preparation which is in such form as to permit the biological activity of the active ingredient (e.g., an isolated antibody as described herein, including, but not limited to an anti-CCL21 antibody) to be effective, and which contains no additional components that would be are unacceptably toxic to a subject to whom the formulation would be administered. Such a formulation can be sterile, i.e., aseptic or free from all living microorganisms and their spores, etc.

The term “package insert” is used to refer to instructions customarily included in commercial packages of therapeutic products, that contain information about the indications, usage, dosage, administration, contraindications and/or warnings concerning the use of such therapeutic products.

The terms “antibody,” “immunoglobulin,” and “Ig” are used interchangeably herein in a broad sense and specifically cover, for example, individual anti-CCL21 antibodies, such as the monoclonal antibodies described herein, (including agonist, antagonist, neutralizing antibodies, full length or intact monoclonal antibodies, peptide fragments of antibodies that maintain antigen binding activity); anti-CCL21 antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain anti-CCL21 antibodies, and fragments of anti-CCL21 antibodies, as described herein. An antibody can be human, humanized, chimeric and/or affinity matured. An antibody may be from other species, for example, mouse, rat, rabbit, etc. The term “antibody” is intended to include a polypeptide product of B cells within the immunoglobulin class of polypeptides that is able to bind to a specific molecular antigen. An antibody is typically composed of two identical pairs of polypeptide chains, wherein each pair has one heavy chain (about 50-70 kDa) and one light chain (about 25 kDa); and wherein the amino-terminal portion of the heavy and light chains includes a variable region of about 100 to about 130 or more amino acids and the carboxy-terminal portion of each chain includes a constant region (See, Borrebaeck (ed.), 1995, Antibody Engineering, Second Ed., Oxford University Press; Kuby, 1997 Immunology, Third Ed., W.H. Freeman and Company, New York). In some aspects, the specific molecular antigen bound by an antibody provided herein includes a CCL21 polypeptide, a CCL21 peptide fragment, or a CCL21 epitope. An antibody or a peptide fragment thereof that binds to a CCL21 antigen can be identified, for example, by immunoassays, BIAcore, or other techniques known to those of skill in the art. An antibody or a fragment thereof binds specifically to a CCL21 antigen when it binds to a CCL21 antigen with higher affinity than to any cross-reactive antigen as determined using experimental techniques, such as radioimmunoassays (RIA) and enzyme linked immunosorbent assays (ELISAs). Typically, a specific or selective binding reaction will be at least twice background signal or noise, and more typically more than 5-10 times background signal or noise. See, e.g., Paul, ed., 1989, Fundamental Immunology Second Edition, Raven Press, New York at pages 332-336 for a discussion regarding antibody specificity.

Antibodies provided herein include, but are not limited to, synthetic antibodies, monoclonal antibodies, recombinantly produced antibodies, multispecific antibodies (including bi-specific antibodies), human antibodies, humanized antibodies, camelized antibodies, chimeric antibodies, intrabodies, anti-idiotypic (anti-Id) antibodies, and functional fragments (e.g., antigen-binding fragments such as CCL21 binding fragments) of any of the above. A binding fragment refers to a portion of an antibody heavy or light chain polypeptide, such as a peptide portion, that retains some or all of the binding activity of the antibody from which the fragment is derived. Non-limiting examples of functional fragments (e.g., antigen-binding fragments such as CCL21 binding fragments) include single-chain Fvs (scFv) (e.g., including monospecific, bispecific, etc.), Fab fragments, F(ab′) fragments, F(ab)₂ fragments, F(ab)₂ fragments, disulfide-linked Fvs (sdFv), Fd fragments, Fv fragments, diabodies, triabodies, tetrabodies and minibodies. In particular, antibodies provided herein include immunoglobulin molecules and immunologically active portions of immunoglobulin molecules, for example, antigen binding domains or molecules that contain an antigen-binding site that binds to a CCL21 antigen, (e.g., one or more complementarity determining regions (CDRs) of an anti-CCL21 antibody). Description of such antibody fragments can be found in, for example, Harlow and Lane, 1989, Antibodies: A Laboratory Manual, Cold Spring Harbor Laboratory, New York; Myers (ed.), Molec. Biology and Biotechnology: A Comprehensive Desk Reference, New York: VCH Publisher, Inc.; Huston et al., 1993, Cell Biophysics, 22:189-224; Pluckthun and Skerra, 1989, Meth. Enzymol., 178:497-515 and in Day, E. D., 1990, Advanced Immunochemistry, Second Ed., Wiley-Liss, Inc., New York, NY. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule. Anti-CCL21 antibodies can be agonistic antibodies or antagonistic antibodies. In some aspects, the anti-CCL21 antibodies can be fully human, such as fully human monoclonal anti-CCL21 antibodies. In some aspects, the anti-CCL21 antibodies can be humanized, such as humanized monoclonal anti-CCL21 antibodies. In some aspects, the antibodies provided herein can be IgG antibodies, or a class (e.g., human IgG1 or IgG4) or subclass thereof, in particular, IgG1 subclass antibodies.

A four-chain antibody unit is a heterotetrameric glycoprotein composed of two identical light (L) chains and two identical heavy (H) chains. In the case of IgGs, the molecular weight of the four-chain (unreduced) antibody unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. At the N-terminus, each H chain has a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its carboxy terminus. The V_{L} domain is aligned with the V_{H} domain, and the C_{L} domain is aligned with the first constant domain of the heavy chain (C_{H1}). Particular amino acid residues are believed to form an interface between the light chain and heavy chain variable domains. The pairing of a V_{H} and V_{L} together forms a single antigen-binding site, although certain V_{H} and V_{L} domains can bind antigen without pairing with a V_{L} or V_{H} domain, respectively. The basic structure of immunoglobulin molecules is understood by those having skill in the art. For example, the structure and properties of the different classes of antibodies may be found in Terr, Abba I. et al., 1994, Basic and Clinical Immunology, 8th edition, Appleton & Lange, Norwalk, CT, page 71 and Chapter 6.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)n can be used (n=1, 2, 3 or 4).

A “fragment antigen-binding fragment (Fab)” is a region of an antibody that binds to antigen. A Fab comprises constant and variable regions from both heavy and light chains.

As used herein, the term “antigen” or “target antigen” is a predetermined molecule to which an antibody can selectively bind. A target antigen can be a polypeptide, peptide, carbohydrate, nucleic acid, lipid, hapten, or other naturally occurring or synthetic compound. In some aspects, a target antigen can be a small molecule. In some aspects, the target antigen can a polypeptide or peptide, e.g., CCL21.

As used herein, the term “antigen binding fragment,” “antigen binding domain,” “antigen binding region,” and similar terms refer to that portion of an antibody which includes the amino acid residues that interact with an antigen and confer on the antibody as binding agent its specificity and affinity for the antigen (e.g., the CDRs of an antibody are antigen binding regions). The antigen binding region can be derived from any animal species, such as rodents (e.g., rabbit, rat, or hamster) and humans. In some aspects, the antigen binding region can be of human origin.

An “isolated” antibody is substantially free of cellular material or other contaminating proteins from the cell or tissue source and/or other contaminant components from which the antibody is derived, or is substantially free of chemical precursors or other chemicals when chemically synthesized. The language “substantially free of cellular material” includes preparations of an antibody in which the antibody is separated from cellular components of the cells from which it is isolated or recombinantly produced. Thus, an antibody that is substantially free of cellular material includes preparations of an antibody that have less than about 30%, 25%, 20%, 15%, 10%, 5%, or 1% (by dry weight) of heterologous protein (also referred to herein as a “contaminating protein”). In some aspects, when the antibody is recombinantly produced, it is substantially free of culture medium, e.g., culture medium represents less than about 20%, 15%, 10%, 5%, or 1% of the volume of the protein preparation. In some aspects, when the antibody is produced by chemical synthesis, it is substantially free of chemical precursors or other chemicals, for example, it is separated from chemical precursors or other chemicals which are involved in the synthesis of the protein. Accordingly such preparations of the antibody have less than about 30%, 25%, 20%, 15%, 10%, 5%, or 1% (by dry weight) of chemical precursors or compounds other than the antibody of interest. Contaminant components can also include, but are not limited to, materials that would interfere with therapeutic uses for the antibody, and can include enzymes, hormones, and other proteinaceous or nonproteinaceous solutes. In some aspects, the antibody is purified (1) to greater than or equal to 95% by weight of the antibody, as determined by the Lowry method (Lowry et al., 1951, J Bio. Chem., 193: 265-275), such as 95%, 96%, 97%, 98%, or 99%, by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or silver stain. Isolated antibody also includes the antibody in situ within recombinant cells since at least one component of the antibody's natural environment will not be present. An isolated antibody is typically prepared by at least one purification step. In some aspects, the antibodies provided herein are isolated.

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods.

As used herein, the term “binds” or “binding” refers to an interaction between molecules including, for example, to form a complex. Illustratively, such interactions embrace non-covalent interactions, including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site of an antibody and its epitope on a target (antigen) molecule, such as CCL21, is the affinity of the antibody or functional fragment for that epitope. The ratio of association (kₒₙ) to dissociation (k_{off}) of an antibody to a monovalent antigen (kₒₙ/k_{off}) is the association constant Kₐ, which is a measure of affinity. The value of K varies for different complexes of antibody and antigen and depends on both kₒₙ and k_{off}. The association constant Kₐ for an antibody provided herein may be determined using any method provided herein or any other method known to those skilled in the art. The affinity at one binding site does not always reflect the true strength of the interaction between an antibody and an antigen. When complex antigens containing multiple, repeating antigenic determinants come into contact with antibodies containing multiple binding sites, the interaction of antibody with antigen at one site will increase the probability of an interaction at a second binding site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K d). Affinity can be measured by common methods known in the art, including those described herein. Low-affinity antibodies generally bind antigen slowly and tend to dissociate readily, while high-affinity antibodies generally bind antigen faster and tend to remain bound longer to antigen. A variety of methods for measuring binding affinity are known in the art, any of which may be used for purposes of the present disclosure. Specific illustrative aspects include the following: In some aspects, the “K_{d}” or “K_{d} value” is measured by assays known in the art, for example, by a binding assay. The K_{d} can be measured in a radiolabeled antigen binding assay (RIA), for example, performed with the Fab portion of an antibody of interest and its antigen (Chen, et al., 1999, J Mol. Biol., 293:865-881). The K_{d} or K_{d} value may also be measured by using surface plasmon resonance (SPR) assays (by BIAcore) using, for example, a BIAcore™-2000 or a BIAcore™-3000 (BIAcore, Inc., Piscataway, NJ), or by biolayer interferometry (BLI) using, for example, the OctetQK384 system (ForteBio, Menlo Park, CA), or by quartz crystal microbalance (QCM) technology. An “on-rate” or “rate of association” or “association rate” or “kₒₙ” can also be determined with the same surface plasmon resonance or biolayer interferometry techniques described above, using, for example, a BIAcore™-2000 or a BIAcore™-3000 (BIAcore, Inc., Piscataway, NJ), or the OctetQK384 system (ForteBio, Menlo Park, CA).

Disclosed herein are isolated antibodies including, but not limited to, anti-CCL21 antibodies, antibodies that specifically bind to CCL21, antibodies that are specific for CCL21, antibodies that specifically bind to a CCL21 epitope, antibodies that selectively bind to a CCL21 epitope, and antibodies that preferentially binds to CCL21. The terms “anti-CCL21 antibody,” “anti-CCL21 monoclonal antibody”, “monoclonal CCL21”, “an antibody that specifically binds to CCL21,” or “antibody that is specific for CCL21,” “antibodies that specifically bind to a CCL21 epitope,” “an antibody that selectively binds to CCL21,” “antibodies that selectively bind to a CCL21 epitope,” “an antibody that preferentially binds to CCL21”, and analogous terms refer to antibodies capable of binding CCL21, i.e., WT CCL21, with sufficient affinity and specificity, particularly compared with mutants of CCL21.

By “specifically binds” is meant that an antibody recognizes and physically interacts with its cognate antigen (for example, CCL21) and does not significantly recognize and interact with other antigens; such an antibody may be a polyclonal antibody or a monoclonal antibody, which are generated by techniques that are well known in the art.

“Preferential binding” of the anti-CCL21 antibodies as provided herein may be determined or defined based on the quantification of fluorescence intensity of the antibodies' binding to CCL21, i.e., CCL21 polypeptide, or CCL21 WT, or CCL21 expressed on cells versus an appropriate control, such as binding to variant CCL21, or to cells expressing a variant form of CCL21, for example, molecularly engineered cells, cell lines or tumor cell isolates. Preferential binding of an anti-CCL21 antibody as described to a CCL21 WT-expressing cell is indicated by a measured fluorescent binding intensity (MFI) value, as assessed by cell flow cytometry, of at least 2-fold, at least 3-fold, at least 4-fold, at least 5-fold, at least 6-fold, at least 7-fold, at least 8-fold, at least 10-fold, at least 15-fold, at least 20-fold or greater, as compared with binding of the antibody to a mutant CCL21 polypeptide or a mutant CCL21-expressing cell, wherein the antibody to be assayed is directly or indirectly detectable by a fluorescent label or marker, such as FITC. In some aspects, the antibody to be assayed is directly labeled with a fluorescent marker, such as FITC. In some aspects, an anti-CCL21 antibody that preferentially or selectively binds CCL21 exhibits an MFI value of from 1.5-fold to 25-fold, or from 2-fold to 20-fold, or from 3-fold to 15-fold, or from 4-fold to 8-fold, or from 2-fold to 10-fold, or from 2-fold to 5-fold or more greater than the MFI value of the same antibody for binding a CCL21 or a CCL21 variant. Fold-fluorescence intensity values between and equal to all of the foregoing are intended to be included. In some aspects, the anti-CCL21 antibodies specifically and preferentially bind to a CCL21 polypeptide, such as a CCL21 antigen, peptide fragment, or epitope (e.g., human CCL21 such as a human CCL21 polypeptide, antigen or epitope). An antibody that specifically binds to CCL21, (e.g., wild type human CCL21) can bind to the extracellular domain (ECD) or a peptide derived from the ECD of CCL21. An antibody that specifically binds to a CCL21 antigen (e.g., human CCL21) can be cross-reactive with related antigens (e.g., cynomolgus (cyno) CCL21). In some aspects, an antibody that specifically binds to a CCL21 antigen does not cross-react with other antigens. An antibody that specifically binds to a CCL21 antigen can be identified, for example, by immunofluorescence binding assays, immunohistochemistry assay methods, immunoassay methods, Biacore, or other techniques known to those of skill in the art.

In some aspects, an antibody that binds to CCL21, as described herein, has a dissociation constant (K_{d}) of less than or equal to 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 0.9 nM, nM, 0.7 nM, 0.6 nM, 0.5 nM, 0.4 nM, 0.3 nM, 0.2 nM, or 0.1 nM, and/or is greater than or equal to 0.1 nM. In some aspects, an antibody that binds to CCL21, as described herein, has a dissociation constant (K_{d}) of less than or equal to 50 nM, 45 nM, 44 nM, 43 nM, 42 nM, 41 nM, 40 nM, 35 nM, 30 nM, 25 nM, 24 nM, 23 nM, 22 nM, 21 nM, and/or is greater than or equal to 20 nM. In some aspects, an anti-CCL21 antibody binds to an epitope of CCL21 that is conserved among CCL21 proteins from different species (e.g., between human and mouse CCL21). An antibody binds specifically to a CCL21 antigen when it binds to a CCL21 antigen with higher affinity than to any cross reactive antigen as determined using experimental techniques, such as radioimmunoassays (RIA) and enzyme linked immunosorbent assays (ELISAs). Typically a specific or selective reaction will be at least twice background signal or noise and can be more than 10 times background. See, e.g., Paul, ed., 1989, Fundamental Immunology Second Edition, Raven Press, New York at pages 332-336 for a discussion regarding antibody specificity. In some aspects, the extent of binding of the antibody to a “non-target” protein will be less than about 10% of the binding of the antibody to its particular target protein, for example, as determined by fluorescence activated cell sorting (FACS) analysis or radioimmunoprecipitation (RIA).

As used herein, in reference to an antibody, the term “heavy (H) chain” refers to a polypeptide chain of about 50-70 kDa, wherein the amino-terminal portion includes a variable (V) region (also called V domain) of about 115 to 130 or more amino acids and a carboxy-terminal portion that includes a constant (C) region. The constant region (or constant domain) can be one of five distinct types, (e.g., isotypes) referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes (e.g., isotypes) of antibodies, namely, IgA, IgD, IgE, IgG and IgM, respectively, including four subclasses of IgG, namely IgG1, IgG2, IgG3 and IgG4. An antibody heavy chain can be a human antibody heavy chain.

As used herein in reference to an antibody, the term “light (L) chain” refers to a polypeptide chain of about 25 kDa, wherein the amino-terminal portion includes a variable domain of about 100 to about 110 or more amino acids and a carboxy-terminal portion that includes a constant region. The approximate length of a light chain (both the V and C domains) is 211 to 217 amino acids. There are two distinct types of light chains, referred to as kappa (κ) and lambda (λ), based on the amino acid sequence of the constant domains. Light chain amino acid sequences are well known in the art. An antibody light chain can be a human antibody light chain.

As used herein, the term “variable (V) region” or “variable (V) domain” refers to a portion of the light (L) or heavy (H) chains of an antibody polypeptide that is generally located at the amino-terminus of the L or H chain. The H chain V domain has a length of about 115 to 130 amino acids, while the L chain V domain is about 100 to 110 amino acids in length. The H and L chain V domains are used in the binding and specificity of each particular antibody for its particular antigen. The V domain of the H chain can be referred to as “VH.” The V region of the L chain can be referred to as “VL.” The term “variable” refers to the fact that certain segments of the V domains differ extensively in sequence among different antibodies. While the V domain mediates antigen binding and defines specificity of a particular antibody for its particular antigen, the variability is not evenly distributed across the 110-amino acid span of antibody V domains. Instead, the V domains consist of less variable (e.g., relatively invariant) stretches called framework regions (FRs) of about 15-30 amino acids separated by shorter regions of greater variability (e.g., extreme variability) called “hypervariable regions” or “complementarity determining regions” (CDRs) that are each about 9-12 amino acids long or 3-17 amino acids long. The V domains of antibody H and L chains each comprise four FRs, largely adopting a β sheet configuration, connected by three hypervariable regions, called, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., 1991, Sequences of Proteins of Immunological Interest, 5th Ed. Public Health Service, National Institutes of Health, Bethesda, MD). The C domains are not involved directly in binding an antibody to an antigen, but exhibit various effector functions, such as antibody dependent cellular cytotoxicity (ADCC) and complement dependent cytotoxicity (CDC). The V domains differ extensively in sequence among different antibody classes or types. The variability in sequence is concentrated in the CDRs, which are primarily responsible for the interaction of the antibody with antigen. In some aspects, the variable domain of an antibody is a human or humanized variable domain.

As used herein, the terms “complementarity determining region,” “CDR,” “hypervariable region,” “HVR,” and “HV” are used interchangeably. A “CDR” or “complementarity determining region” is a region of hypervariability interspersed within regions that are more conserved, termed “framework regions” (FR). A “CDR” refers to one of three hypervariable regions (H1, H2 or H3) within the non-framework region of the antibody VH β-sheet framework, or to one of three hypervariable regions (L1, L2 or L3) within the non-framework region of the antibody VL β-sheet framework. The term, when used herein, refers to the regions of an antibody V domain that are hypervariable in sequence and/or form structurally defined loops. Generally, antibodies comprise six hypervariable regions: three (H1, H2, H3) in the VH domain and three (L1, L2, L3) in the VL domain. Accordingly, CDRs are typically highly variable sequences interspersed within the framework region sequences of the V domain. “Framework” or “FR” residues are those variable region residues flanking the CDRs. FR residues are present, for example, in chimeric, humanized, human, domain antibodies, diabodies, linear antibodies, and bispecific antibodies.

A number of hypervariable region delineations are in use and are encompassed herein. CDR regions are well known to those skilled in the art and have been defined by, for example, Kabat as the regions of most hypervariability within the antibody V domains (Kabat et al., 1977, J. Biol. Chem., 252:6609-6616; Kabat, 1978, Adv. Prot. Chem., 32:1-75). The Kabat CDRs are based on sequence variability and are the most commonly used (see, e.g., Kabat et al., 1991, Sequences of Proteins of Immunological Interest, 5th Ed. Public Health Service, National Institutes of Health, Bethesda, MD). CDR region sequences also have been defined structurally by Chothia as those residues that are not part of the conserved β-sheet framework, and thus are able to adopt different conformations (Chothia et al., 1987, J. Mol. Biol., 196:901-917). Chothia refers instead to the location of the structural loops. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both numbering systems and terminologies are well recognized in the art.

Recently, a universal numbering system has been developed and widely adopted, ImMunoGeneTics (IMGT) Information System® (Lafranc et al., 2003, Dev. Comp. Immunol., 27(1):55-77). IMGT is an integrated information system specializing in immunoglobulins (Ig), T cell receptors (TR) and the major histocompatibility complex (MHC) of human and other vertebrates. Herein, the CDRs are referred to in terms of both the amino acid sequence and the location within the light or heavy chain. As the “location” of the CDRs within the structure of the immunoglobulin V domain is conserved between species and present in structures called loops, by using numbering systems that align variable domain sequences according to structural features, CDR and framework residues and are readily identified. This information can be used in grafting and in the replacement of CDR residues from immunoglobulins of one species into an acceptor framework from, typically, a human antibody. An additional numbering system (AHon) has been developed by Honegger et al., 2001, J. Mol. Biol., 309: 657-670. Correspondence between the numbering system, including, for example, the Kabat numbering and the IMGT unique numbering system, is well known to one skilled in the art (see, e.g., Kabat, Id; Chothia et al., Id.; Martin, 2010, Antibody Engineering, Vol. 2, Chapter 3, Springer Verlag; and Lefranc et al., 1999, Nuc. Acids Res., 27:209-212).

CDR region sequences have also been defined by AbM, Contact and IMGT. The AbM hypervariable regions represent a compromise between the Kabat CDRs and Chothia structural loops, and are used by Oxford Molecular's AbM antibody modeling software (see, e.g., Martin, 2010, Antibody Engineering, Vol. 2, Chapter 3, Springer Verlag). The “contact” hypervariable regions are based on an analysis of the available complex crystal structures. The residues from each of these hypervariable regions or CDRs are noted below.

Exemplary delineations of CDR region sequences are illustrated in Table 2. The positions of CDRs within a canonical antibody variable region have been determined by comparison of numerous structures (Al-Lazikani et al., 1997, J. Mol. Biol., 273:927-948); Morea et al., 2000, Methods, 20:267-279). Because the number of residues within a hypervariable region varies in different antibodies, additional residues relative to the canonical positions are conventionally numbered with a, b, c and so forth next to the residue number in the canonical variable region numbering scheme (Al-Lazikani et al., Id). Such nomenclature is similarly well known to those skilled in the art.

TABLE 2
Exemplary Delineations of CDR Region Sequences

| | IMGT | Kabat |
|---|---|---|
| V_{H} CDR1 | 28-35 | 31-35 |
| V_{H} CDR2 | 51-58 | 47-56 |
| V_{H} CDR3 | 97-103 | 99-103 |
| V_{L} CDR1 | 27-32 | 24-34 |
| V_{L} CDR2 | 50-52 | 50-56 |
| V_{L} CDR3 | 89-97 | 89-97 |

An “affinity matured” antibody is one with one or more alterations (e.g., amino acid sequence variations, including changes, additions and/or deletions) in one or more HVRs thereof that result in an improvement in the affinity of the antibody for antigen, compared to a parent antibody which does not possess those alteration(s). In some aspects, affinity matured antibodies will have nanomolar or even picomolar affinities for the target antigen, such as CCL21. Affinity matured antibodies are produced by procedures known in the art. For reviews, see Hudson and Souriau, 2003, Nature Medicine, 9:129-134; Hoogenboom, 2005, Nature Biotechnol., 23:1105-1116; Quiroz and Sinclair, 2010, Revista Ingeneria Biomedia, 4: 39-51.

A “chimeric” antibody is one in which a portion of the H and/or L chain, e.g., the V domain, is identical with or homologous to a corresponding amino acid sequence in an antibody derived from a particular species or belonging to a particular antibody class or subclass, while the remainder of the chain(s), e.g., the C domain, is identical with or homologous to corresponding sequences in antibodies derived from another species or belonging to another antibody class or subclass, as well as a fragment of such an antibody, so long as it exhibits the desired biological activity (see, e.g., U.S. Pat. No. 4,816,567; and Morrison et al., 1984, Proc. Natl. Acad. Sci. USA, 81:6851-6855).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033). The terms “human antibody” and “fully human antibody” are used interchangeably herein and refer to an antibody that possesses an amino acid sequence which corresponds to that of an antibody produced by a human and/or has been made using any of the techniques for making human antibodies as practiced by those skilled in the art. This definition of a human antibody specifically excludes a humanized antibody comprising non-human antigen-binding residues. Human antibodies can be produced using various techniques known in the art, including phage-display libraries (Hoogenboom et al., 1991, J. Mol. Biol., 227:381; Marks et al., 1991, J. Mol. Biol., 222:581 and yeast display libraries (Chao et al., 2006, Nature Protocols, 1:755-768). Also available for the preparation of human monoclonal antibodies are methods described in Cole et al., 1985 Monoclonal Antibodies and Cancer Therapy, Alan R. Liss, p. 77; Boemer et al., 1991, J. Immunol., 147(1):86-95. See also van Dijk et al., 2001, Curr. Opin. Pharmacol., 5: 368-74. Human antibodies can be prepared by administering an antigen to a transgenic animal whose endogenous Ig loci have been disabled, e.g., a mouse, and that has been genetically modified to harbor human immunoglobulin genes which encode human antibodies, such that human antibodies are generated in response to antigenic challenge (see, e.g., Jakobovits, A., 1995, Curr. Opin. Biotechnol. 6(5):561-566; Brüggemann et al., 1997 Curr. Opin. Biotechnol., 8(4):455-8; and U.S. Pat. Nos. 6,075,181 and 6,150,584 regarding XENOMOUSE™ technology). See also, for example, Li et al., 2006, Proc. Natl. Acad. Sci. USA, 103:3557-3562 regarding human antibodies generated via a human B-cell hybridoma technology. In some aspects, human antibodies comprise a variable region and constant region of human origin. “Fully human” anti-CCL21 antibodies, in some aspects, can also encompass antibodies which bind CCL21 polypeptides and are encoded by nucleic acid sequences which are naturally occurring somatic variants of human germline immunoglobulin nucleic acid sequence. In some aspects, the anti-CCL21 antibodies provided herein are fully human antibodies. The term “fully human antibody” includes antibodies having variable and constant regions corresponding to human germline immunoglobulin sequences as described by Kabat et al. (See Kabat et al., 1991, Sequences of Proteins of Immunological Interest, Fifth Edition, U.S. Department of Health and Human Services, NIH Publication No. 91-3242). The phrase “recombinant human antibody” includes human antibodies that are prepared, expressed, created, or isolated by recombinant means, such as antibodies expressed using a recombinant expression vector transfected into a host cell; antibodies isolated from a recombinant, combinatorial human antibody library; antibodies isolated from an animal (e.g., a mouse or cow) that is transgenic and/or transchromosomal for human immunoglobulin genes (see e.g., Taylor, L. D. et al., 1992, Nucl. Acids Res. 20:6287-6295); or antibodies prepared, expressed, created or isolated by any other means that involves splicing of human immunoglobulin gene sequences to other DNA sequences. Such recombinant human antibodies can have variable and constant regions derived from human germline immunoglobulin sequences (See Kabat et al., 1991, Sequences of Proteins of Immunological Interest, Fifth Edition, U.S. Department of Health and Human Services, NIH Publication No. 91-3242). In some aspects, however, such recombinant human antibodies are subjected to in vitro mutagenesis (or, when an animal transgenic for human Ig sequences is used, in vivo somatic mutagenesis) and, thus, the amino acid sequences of the VH and VL regions of the recombinant antibodies are sequences that, while derived from and related to human germline VH and VL sequences, may not naturally exist within the human antibody germline repertoire in vivo.

As used herein, the term “epitope” is the site(s) or region(s) on the surface of an antigen molecule to which a single antibody molecule binds, such as a localized region on the surface of an antigen, e.g., a CCL21 polypeptide that is capable of being bound by one or more antigen binding regions of an anti-CCL21 antibody. An epitope can be immunogenic and capable of eliciting an immune response in an animal. Epitopes need not necessarily be immunogenic. Epitopes often consist of chemically active surface groupings of molecules such as amino acids or sugar side chains and have specific three dimensional structural characteristics as well as specific charge characteristics. An epitope can be a linear epitope and a conformational epitope. A region of a polypeptide contributing to an epitope can be contiguous amino acids of the polypeptide, forming a linear epitope, or the epitope can be formed from two or more non-contiguous amino acids or regions of the polypeptide, typically called a conformational epitope. The epitope may or may not be a three-dimensional surface feature of the antigen. In some aspects, a CCL21 epitope is a three-dimensional surface feature of a CCL21 polypeptide. In some aspects, a CCL21 epitope is linear feature of a CCL21 polypeptide.

An antibody binds “an epitope” or “essentially the same epitope” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies to antigen is measured using a detectable signal, e.g., radioactive, fluorescent or enzyme labels.

The term “compete” when used in the context of anti-CCL21 antibodies that compete for the same epitope or binding site on a CCL21 target protein or peptide thereof means competition as determined by an assay in which the antibody under study, or binding fragment thereof, prevents, blocks, or inhibits the specific binding of a reference molecule (e.g., a reference ligand, or reference antigen binding protein, such as a reference antibody) to a common antigen (e.g., CCL21 or a fragment thereof). Numerous types of competitive binding assays can be used to determine if a test antibody competes with a reference antibody for binding to CCL21 (e.g., human CCL21). Examples of assays that can be employed include solid phase direct or indirect radioimmunoassay (RIA); solid phase direct or indirect enzyme immunoassay (EIA); sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using labeled iodine (1125 label) (see, e.g., Morel et al., 1988, Molec. Immunol. 25:7-15); solid phase direct biotin-avidin EIA (see, e.g., Cheung, et al., 1990s, Virology 176:546-552); and direct labeled RIA (Moldenhauer et al., 1990, Scand. J. Immunol. 32:77-82). Typically, such an assay involves the use of a purified antigen (e.g., CCL21) bound to a solid surface, or cells bearing either of an unlabeled test antigen binding protein (e.g., test anti-CCL21 antibody) or a labeled reference antigen binding protein (e.g., reference anti-CCL21 antibody). Competitive inhibition can be measured by determining the amount of label bound to the solid surface or cells in the presence of a known amount of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference antibody causing steric hindrance to occur. Additional details regarding methods for determining competitive binding are described herein. Usually, when a competing antibody protein is present in excess, it will inhibit specific binding of a reference antibody to a common antigen by at least 15%, or at least 20%, for example, without limitation, 25%, 30%, 35%, 40%, 45%, 50%, 55%, 60%, 65%, 70% or 75% or greater, as well as percent amounts between the amounts stated. In some aspects, binding can be inhibited by at least 80%, 85%, 90%, 95%, 96% or 97%, 98%, 99% or more.

As used herein, the term “blocking” antibody or an “antagonist” antibody refers to an antibody that prevents, inhibits, blocks, or reduces biological or functional activity of the antigen to which it binds. Blocking antibodies or antagonist antibodies can substantially or completely prevent, inhibit, block, or reduce the biological activity or function of the antigen. For example, a blocking anti-CCL21 antibody can prevent, inhibit, block, or reduce the binding interaction between CCL21 and CCR7, thus preventing, blocking, inhibiting, or reducing the immune system functions associated with the CCR7/CCL21 interaction. The terms block, inhibit, and neutralize are used interchangeably herein and refer to the ability of the anti-CCL21 antibodies to prevent or otherwise disrupt or reduce the CCL21/CCR7 interaction.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Chemokines are a family of small proteins that share structural and functional elements. They share cysteine-mediated covalent bonds in the amino terminus and mediation of leukocyte migration (Hughes C E, Nibbs R J B. FEBS J. 2018; 285(16):2944-2971; and Hernández-Ruiz M, Zlotnik A. Mucosal Chemokines. J Interferon Cytokine Res. 2017; 37(2):62-70). Chemokines and their receptors are important in many types of human diseases, playing important roles in inflammatory tissue destruction seen in atherosclerosis, adult respiratory distress syndrome, cerebral vascular events, and myocardial infarction (Bryant V L, Slade C A. Immunol Cell Biol. 2015; 93(4):364-7; Kolaczkowska E, Kubes P. Nat Rev Immunol. 2013; 13(3):159-75; and Sakai Y, Kobayashi M. Pathol Int. 2015; 65(7):344-54). It has been shown that chemokines can mediate the inhibition of hematopoiesis seen during systemic inflammation (Broxmeyer H E, et al. Ann N Y Acad Sci. 1999; 872:142-62). In addition, chemokines can promote the aberrant migration of leukocytes into target organs during autoimmune diseases, such as lupus, rheumatoid arthritis, and inflammatory bowel disease (Sakai Y, Kobayashi M. Pathol Int. 2015; 65(7):344-54; Wang L, et al. J Intern Med. 2015; 278(4):369-95; and Singh U P, et al. Chemokine and cytokine levels in inflammatory bowel disease patients. Cytokine. 2016; 77:44-9).

The CC chemokine designated CCL21 is a potent chemo-attractant for naïve T-cells and immature dendritic cells (Hromas R, et al. J Immunol. 1997; 159:2554-2558; Hedrick J A, Zlotnik A. J Immunol. 1997; 159:1589-1593; and Nagira M, et al. J Biol Chem. 1997; 272:19518-19524). CCL21 directed migration of naive B-cells and natural killer (NK) cells, although to a lesser extent than T-cells. It does not direct migration of monocytes or granulocytes (Hromas R, et al. J Immunol. 1997; 159:2554-2558; Hedrick J A, Zlotnik A. J Immunol. 1997; 159:1589-1593; and Nagira M, et al. J Biol Chem. 1997; 272:19518-19524). CCL21 also promotes naïve lymphocyte adhesion to the endothelium of small venules, most notably in lymph nodes where naïve lymphocytes can be presented with antigen, but also in primary inflamed tissues as well (Comerford I, et al. Cytokine Growth Factor Rev. 2013; 24(3):269-83; Förster R, et al. Nat Rev Immunol. 2008; 8(5):362-71; and Gunn M D, et al. Proc Natl Acad Sci USA. 1998; 95:258-263). The expression of CCL21 in the high endothelial venules of lymph nodes therefore mediates naïve T-cell trafficking to secondary lymphoid organs for antigen presentation.

In skin autoimmune disease, CCL21 was highly induced in the venule endothelium of the inflamed tissue but not in normal tissue (Christopherson K W 2nd, et al. Blood. 2003; 101(3):801-6). In addition, the vast majority of the infiltrating T-cells expressed CCR7, the receptor for CCL21. This raised the possibility that local auto-antigen presentation in the primary inflamed tissue could be more important than secondary lymphoid tissue auto-antigen presentation in generating local tissue damaging effector T-cells (Comerford I, et al. Cytokine Growth Factor Rev. 2013; 24(3):269-83; Förster R, et al. Nat Rev Immunol. 2008; 8(5):362-71; Gunn M D, et al. Proc Natl Acad Sci USA. 1998; 95:258-263; and Christopherson K W 2nd, et al. Blood. 2003; 101(3):801-6).

While progress has been made, changing the course of inflammatory bowel diseases (IBD) is still problematic because the initiators of the disease are imperfectly defined (Yao D, et al. Inflamm Bowel Dis. 2019; 25(10): 1595-1602; and Zhang Y Z, Li Y Y. World J Gastroenterol. 2014; 20(1):91-9). The goal of interrupting the signaling cascade that leads to the destruction of the intestinal mucosa in these diseases has been hampered not just by the lack of targets but by the ineffectiveness of therapies against the targets that are known (Sakai Y, Kobayashi M. Pathol Int. 2015; 65(7):344-54; Singh U P, et al. Cytokine. 2016; 77:44-9; Yao D, et al. Inflamm Bowel Dis. 2019; 25(10): 1595-1602; Zhang Y Z, Li Y Y. World J Gastroenterol. 2014; 20(1):91-9; Zhang H, et al. Genet Mol Res. 2014; 13(2):3337-45; and Danese S, Fiocchi C. Dig Dis. 2016; 34(1-2):43-50). Since the gut is its own secondary lymphoid organ where naïve lymphocytes can be presented antigen locally instead of maturing in a secondary lymphoid organ and then migrating to target inflamed/infected tissues, an IBD therapeutic target could be interrupting the migration of naïve lymphoid cells into the inflamed gut (Sakai Y, Kobayashi M. Pathol Int. 2015; 65(7):344-54; Wang L, et al. J Intern Med. 2015; 278(4):369-95; Singh U P, et al. Cytokine. 2016; 77:44-9; and Yao D, et al. Inflamm Bowel Dis. 2019; 25(10): 1595-1602). CCL21 expression correlated with induction of ulcerative colitis in mice, and when that colitis was treated CCL21 expression decreased (Singh U P, et al. Cytokine. 2016; 77:44-9; and Zhang H, et al. Genet Mol Res. 2014; 13(2):3337-45). Therefore, it was tested whether blocking CCL21-directed migration of naïve immune cells alters IBD (Danese S, Fiocchi C. Dig Dis. 2016; 34(1-2):43-50; and Fritsch R D, et al. J Immunol 2005; 175 (10) 6489-6497). While there is at least one commercially available neutralizing monoclonal antibody against human CCL21 (R&D Systems, Minneapolis, MN), its activity in immunohistology of endothelial CCL21 in T-cell autoimmune diseases and its activity against Th-cell subsets has not been characterized. In addition, this commercially available neutralizing monoclonal antibody against human CCL21 has not been humanized and thus would not be appropriate for clinical development as a therapeutic agent in T-cell autoimmune diseases.

Although the foregoing disclosure has been described in some detail by way of illustration and example for purposes of clarity of understanding, certain changes and modifications may be practiced within the scope of the appended claims.

Compositions

Anti-CCL21 antibodies. Disclosed herein are isolated antibodies, including, but not limited to, anti-CCL21 antibodies or binding fragments thereof. Disclosed herein are anti-CCL21 antibodies or binding fragments thereof that bind to CCL21. Disclosed herein are anti-CCL21 antibodies or binding fragments thereof that bind to CCL21 and block or inhibit the immune suppressive function of the CCL21/CCR7 interaction (e.g., block or inhibit the binding of CCL21 to CCR7). Disclosed herein are anti-CCL21 antibodies or binding fragments thereof useful in treating autoimmune disorders and preventing graft rejection. Also disclosed herein are anti-CCL21 antibodies or binding fragments thereof useful in the treating cancer and inhibiting or preventing tumor or cancer metastases.

The anti-CCL21 antibodies disclosed herein can be of the IgG, IgM, IgA, IgD, and IgE Ig classes, as well as polypeptides comprising one or more antibody CDR domains that retain antigen binding activity. Illustratively, the anti-CCL21 antibodies may be chimeric, affinity matured, humanized, or human antibodies. In some aspects, the anti-CCL21 antibodies can be monoclonal antibodies. In some aspects, the monoclonal anti-CCL21 antibody can be a humanized antibody. By known means and as described herein, polyclonal or monoclonal antibodies, antibody fragments, binding domains and CDRs (including engineered forms of any of the foregoing) may be created that are specific for CCL21 antigen, one or more of its respective epitopes, or conjugates of any of the foregoing, whether such antigens or epitopes are isolated from natural sources or are synthetic derivatives or variants of the natural protein.

Also disclosed herein are compositions comprising the disclosed isolated antibodies, including, but not limited to anti-CCL21 antibodies. In some aspects, the antibodies disclosed herein can be isolated antibodies. Examples of the CDR sequences and heavy or light chain variable region sequences of anti-CCL21 antibodies are shown in Tables 3, 4 and 5.

Disclosed herein are isolated antibodies comprising a light chain variable region and a heavy chain variable region. In some aspects, the light chain variable region can comprise a complementarity determining region light chain 1 (CDRL1) amino acid sequence of SEQ ID NO: 1; a complementarity determining region light chain 2 (CDRL2) amino acid sequence of SEQ ID NO: 2; and a complementarity determining region light chain 3 (CDRL3) amino acid sequence of SEQ ID NO: 3. In some aspects, the light chain variable region can comprise a complementarity determining region light chain 1 (CDRL1) amino acid sequence of SEQ ID NO: 68; a complementarity determining region light chain 2 (CDRL2) amino acid sequence of SEQ ID NO: 2; and a complementarity determining region light chain 3 (CDRL3) amino acid sequence of SEQ ID NO: 3. In some aspects, the heavy chain variable region can comprise a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10.

Also disclosed herein, are isolated antibodies comprising a light chain variable region amino acid sequence of SEQ ID NO: 7 and a heavy chain variable region amino acid sequence of SEQ ID NO: 14.

In some aspects, any of the antibodies disclosed herein can comprise a light chain variable region amino acid sequence comprising SEQ ID NO: 7. In some aspects, any of the antibodies disclosed herein can comprise a heavy chain variable region amino acid sequence comprising SEQ ID NO: 14. In some aspects, a light chain variable region has an amino acid sequence that is at least 90% identical to amino acid sequence SEQ ID NO: 7. In some aspects, a heavy chain variable region has an amino acid sequence that is at least 90% identical to amino acid sequence SEQ ID NO: 14.

Also disclosed herein are isolated antibodies comprising a light chain variable region amino acid sequence of SEQ ID NOs: 63, 64, 65 or 66 and a heavy chain variable region amino acid sequence of SEQ ID NOs: 59, 60, 62 or 62.

Also disclosed herein, are isolated antibodies comprising a light chain variable region amino acid sequence of SEQ ID NO: 64 and a heavy chain variable region amino acid sequence of SEQ ID NO: 60.

In some aspects, any of the antibodies disclosed herein can comprise a light chain variable region amino acid sequence comprising SEQ ID NO: 64. In some aspects, any of the antibodies disclosed herein can comprise a heavy chain variable region amino acid sequence comprising SEQ ID NO: 60. In some aspects, a light chain variable region has an amino acid sequence that is at least 90% identical to amino acid sequence SEQ ID NO: 64. In some aspects, a heavy chain variable region has an amino acid sequence that is at least 90% identical to amino acid sequence SEQ ID NO: 60.

Also disclosed herein are isolated antibodies comprising any of the light chain variable region amino acid sequences of SEQ ID NOs: 63, 64, 65 or 66 and any of the heavy chain variable region amino acid sequences of SEQ ID NOs: 59, 60, 62 or 62.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 1; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region and a heavy chain variable region, wherein the light chain variable region comprises a complementarity determining region light chain 1 (CDRL1) amino acid sequence of SEQ ID NO: 68; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10, wherein one or more of the CDRL1, CDRL2, CDRL3, CDRH1, CDRH2, or CDRH3 comprise 1, 2, 3, 4, or 5 conservative amino acid substitutions.

Disclosed herein are isolated antibodies comprising a light chain variable region amino acid sequence of SEQ ID NO: 7 and a heavy chain variable region amino acid sequence of SEQ ID NO: 14, wherein the isolated antibody comprises 1, 2, 3, 4, or 5 conservative amino acid substitutions in the light or heavy chain variable region amino acid sequences.

Disclosed herein are isolated antibodies comprising a light chain variable region amino acid sequence of SEQ ID NOs: 63, 64, 65 or 66 and a heavy chain variable region amino acid sequence of SEQ ID NOs: 59, 60, 62 or 62, wherein the isolated antibody comprises 1, 2, 3, 4, or 5 conservative amino acid substitutions in the light or heavy chain variable region amino acid sequences.

Disclosed herein are isolated antibodies comprising a light chain variable region amino acid sequence of SEQ ID NO: 64 and a heavy chain variable region amino acid sequence of SEQ ID NO: 60, wherein the isolated antibody comprises 1, 2, 3, 4, or 5 conservative amino acid substitutions in the light or heavy chain variable region amino acid sequences.

TABLE 3
Exemplary Amino Acid Sequences of anti-CCL21
antibody

| SEQ | | Des- |
|---|---|---|
| ID NO: | Sequence | cription |
| 1 | HASQNINVWLS | CDRL1, |
| | | Kabat |
| 2 | KASNLHT | CDRL2, |
| | | Kabat |
| 3 | HQGHSFPLT | CDRL3, |
| | | Kabat |
| 4 | QNINVW | CDRL1, |
| | | IMGT |
| 5 | KAS | CDRL2, |
| | | IMGT |
| 6 | HQGHSFPLT | CDRL3, |
| | | IMGT |
| 7 | DIQMNQSPSSLSASLGDTVTITCHASQN | Kappa |
| | INVWLSWYQQKPGNIPKLLIYKASNLHT | Light |
| | GVPSRFSGSGSGTGFTLTISSLHPEDIATY | Chain |
| | YCHQGHSFPLTFGAGTKLELK | Variable |
| | | Domain, |
| | | Kabat |
| 58 | DIQMNQSPSSLSASLGDTVTITCHASQN | Kappa |
| | INVWLSWYQQKPGNIPKLLIYKASNLH | Light |
| | TGVPSRFSGSGSGTGFTLTISSLHPEDIA | Chain |
| | TYYCHQGHSFPLTFGAGTKLELK | Variable |
| | | Domain, |
| | | IMGT |
| 8 | DFYLN | CDRH1, |
| | | Kabat |
| 9 | WIDPENGDTEYAPKFQG | CDRH2, |
| | | Kabat |
| 10 | FDYDY | CDRH3, |
| | | Kabat |
| 11 | GFNIKDFY | CDRH1, |
| | | IMGT |
| 12 | IDPENGDT | CDRH2, |
| | | IMGT |
| 13 | NAFDYDY | CDRH3, |
| | | IMGT |
| 14 | EVQLQQSGAEIVRSGASVRLSCTASGF | Heavy |
| | NIKDFYLNWVKQRPEQGLDWIGWIDP | Chain |
| | ENGDTEYAPKFQGKATMTADTSSNTI | Variable |
| | YLHLSSLTSEDTAVYYCNAFDYDYW | Domain, |
| | GQGTTLTVSS | Kabat |
| 15 | EVQLQQSGAEIVRSGASVRLSCTASGF | Heavy |
| | NIKDFYLNWVKQRPEQGLDWIGWIDP | Chain |
| | ENGDTEYAPKFQGKATMTADTSSNTI | Variable |
| | YLHLSSLTSEDTAVYYCNAFDYDYW | Domain, |
| | GQGTTLTVSS | IMGT |
| 68 | RASQNINVWLS | hCDRL1 |
| 69 | QASQNINVWLS | hCDRL1 |
| 70 | WIDPENGDTEYAPGFTG | hCDRH2 |

TABLE 4
Exemplary Delineations of CDR Region Sequences

| Description | IMGT | Kabat |
|---|---|---|
| VH CDR1 | GFNIKDFY (SEQ ID NO: 11) | DFYLN (SEQ ID NO: 8) |
| VH CDR2 | IDPENGDT (SEQ ID NO: 12) | WIDPENGDTEYAPKFQG |
| | | (SEQ ID NO: 9) |
| VH CDR3 | NAFDYDY (SEQ ID NO: 13) | FDYDY (SEQ ID NO: 10) |
| VL CDR1 | QNINVW (SEQ ID NO: 4) | HASQNINVWLS |
| | | (SEQ ID NO: 1) |
| VL CDR2 | KAS (SEQ ID NO: 5) | KASNLHT (SEQ ID NO: 2) |
| VL CDR3 | HQGHSFPLT (SEQ ID NO: 6) | HQGHSFPLT (SEQ ID NO: 3) |
| VL CDR1 | RASQNINVWLS (SEQ ID NO: | RASQNINVWLS (SEQ ID NO: 68) |
| | 68) | |
| VL CDR1 | QASQNINVWLS (SEQ ID NO: | QASQNINVWLS (SEQ ID NO: 69) |
| | 69) | |

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, a “variant CDR” can be a sequence that contains 1, 2, 3, 4 or 5 amino acid changes as compared to the parent or reference CDR of the invention, and shares or improves biological function, specificity and/or activity of the parent CDR.

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 1, 2, 3, 4, 5, 6, 8, 9, 10, 11, 12, 13, 68, 69 and/or 70. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 1, 2, 3, 4, 5, 6, 8, 9, 10, 11, 12, 13, 68, 69, and/or 70. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) non-polar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, an isolated replacement of a leucine with an isoleucine or valine, an aspartate with a glutamate, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physicochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteines on a peptide or in one of the disclosed antibodies or fragments thereof may be susceptible to forming disulfide linkages unless a reducing agent such as dithiothreitol (DTT) is added to the buffer or the cysteines can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein.

By “Fv” or “Fv fragment” or “Fv region” as used herein is meant a polypeptide that comprises the VL and VH domains of a single antibody.

By “framework” as used herein is meant the region of an antibody variable domain exclusive of those regions defined as CDRs. Each antibody variable domain framework can be further subdivided into the contiguous regions separated by the CDRs (FR1, FR2, FR3 and FR4).

Disclosed herein are isolated antibodies comprising a light chain variable region, wherein the light chain variable region comprises a variant complementarity determining region light chain 1 (CDRL1), positions 24-34 of SEQ ID NOs: 7, 63, 64, 65 or 66. In some aspects, the variant CDRL1 can comprise one or two amino acid substitutions when compared to positions 24-34 of SEQ ID NOs: 7, 63, 64, 65 or 66. Also, disclosed herein are isolated antibodies comprising a light chain variable region, wherein the light chain variable region comprises a variant CDRL2, positions 50-56 of SEQ ID NOs: 7, 63, 64, 65 or 66. In some aspects, the variant CDRL2 can comprise one or two amino acid substitutions when compared to positions 50-56 of SEQ ID NOs: 7, 63, 64, 65 or 66. Further disclosed herein are isolated antibodies comprising a light chain variable region, wherein the light chain variable region comprises a variant CDRL3, 89-97 positions of SEQ ID NOs: 7, 63, 64, 65 or 66. In some aspects, the variant CDRL3 can comprise one or two amino acid substitutions when compared to positions 89-97 of SEQ ID NOs: 7, 63, 64, 65 or 66.

Disclosed herein are isolated antibodies comprising a heavy chain variable region, wherein the heavy chain variable region comprises a variant complementarity determining region heavy chain 1 (CDRH1), positions 31-35 of SEQ ID NOs: 14, 59, 60, 61 or 62. In some aspects, the variant CDRH1 can comprise one or two amino acid substitutions when compared to positions 31-35 of SEQ ID NOs: 14, 59, 60, 61 or 62. Also disclosed herein are isolated antibodies comprising a heavy chain variable region, wherein the heavy chain variable region comprises a variant CDRH2, positions 50-66 of SEQ ID NOs: 14, 59, 60, 61 or 62. In some aspects, the variant CDRH2 can comprise one or two amino acid substitutions when compared to positions 50-66 of SEQ ID NOs: 14, 59, 60, 61 or 62. Further disclosed herein are isolated antibodies comprising a heavy chain variable region, wherein the heavy chain variable region can comprise a variant CDRH3, positions 99-103 of SEQ ID NOs: 14, 59, 60, 61 or 62. In some aspects, the variant CDRH3 can comprise one or two amino acid substitutions when compared to positions 99-103 of SEQ ID NOs: 14, 59, 60, 61 or 62.

Disclosed herein are isolated antibodies comprising a light chain variable region and a heavy chain variable region, wherein the light chain variable region comprises a variant complementarity determining region light chain 1 (CDRL1) comprising positions 24-34 of SEQ ID NOs: 7, 63, 64, 65 or 66, wherein the variant CDRL1 comprises one or two amino acid substitutions when compared to positions 24-34 of SEQ ID NOs: 7, 63, 64, 65 or 66; a variant complementarity determining region light chain 2 (CDRL2) comprising positions 50-56 of SEQ ID NOs: 7, 63, 64, 65 or 66, wherein the variant CDRL2 comprises one or two amino acid substitutions when compared to positions 50-56 of SEQ ID NOs: 7, 63, 64, 65 or 66; and a variant complementarity determining region light chain 3 (CDRL3) comprising 89-97 positions of SEQ ID NOs: 7, 63, 64, 65 or 66, wherein the variant CDRL3 comprises one or two amino acid substitutions when compared to positions 89-97 of SEQ ID NOs: 7, 63, 64, or 66; wherein the heavy chain variable region comprises a variant complementarity determining region heavy chain 1 (CDRH1) comprising positions 31-35 of SEQ ID NOs: 14, 59, 60, 61 or 62, wherein the variant CDRH1 comprises one or two amino acid substitutions when compared to positions 31-35 of SEQ ID NOs: 14, 59, 60, 61 or 62; a variant complementarity determining region heavy chain 2 (CDRH2) comprising positions 50-66 of SEQ ID NOs: 14, 59, 60, 61 or 62, and wherein the variant CDRH2 comprises one or two amino acid substitutions when compared to positions 50-66 of SEQ ID NOs: 14, 59, 60, 61 or 62; and a variant complementarity determining region heavy chain 3 (CDRH3) comprising positions 99-103 of SEQ ID NOs: 14, 59, 60, 61 or 62, wherein the variant CDRH3 comprises one or two amino acid substitutions when compared to positions 99-103 of SEQ ID NOs: 14, 59, 60, 61 or 62.

In some aspects, the amino acid substitution of the variant CDRL1 is at positions 24, 29, 30, 31, 32 of SEQ ID NOs: 7, 63, 64, 65 or 66 or a combination thereof. In some aspects, the amino acid substitution of the variant CDRL1 at positions 24, 29, 30, 31, 32 of SEQ ID NOs: 7, 63, 64, 65 or 66 or a combination thereof can be tryptophan, valine, leucine, glutamine, or isoleucine. In some aspects, the amino acid substitution of the variant CDRL2 is at positions 50, 51, 52, 53, 54, 55, 56 of SEQ ID NOs: 7, 63, 64, 65 or 66 or a combination thereof. In some aspects, the amino acid substitution of the variant CDRL2 at positions 50, 51, 52, 53, 54, 55, 56 of SEQ ID NOs: 7, 63, 64, 65 or 66 or a combination thereof can be arginine, leucine, threonine, glutamine, serine, or tryptophan. In some aspects, the amino acid substitution of the variant CDRL3 can be at positions 90, 93, 96, 97 of SEQ ID NOs: 7, 63, 64, 65 or 66 or a combination thereof. In some aspects, the amino acid substitution of variant CDRL3 at positions 90, 93, 96, 97 of SEQ ID NOs: 7, 63, 64, 65 or 66 or a combination thereof can be asparagine, threonine, isoleucine, or serine. In some aspects, the amino acid substitution of the variant CDRH1 can be at positions 31, 34, 35 of SEQ ID NOs: 14, 59, 60, 61 or 62 or a combination thereof. In some aspects, the amino acid substitution of the variant CDRH1 at positions 31, 34, 35 of SEQ ID NOs: 14, 59, 60, 61 or 62 or a combination thereof can be glutamic acid, isoleucine, or glycine. In some aspects, the amino acid substitution of the variant CDRH2 can be at positions 51, 52, 53, 54, 55, 59, 63, 65 of SEQ ID NOs: 14, 59, 60, 61 or 62 or a combination thereof. In some aspects, the amino acid substitution of the variant CDRH2 at positions 51, 52, 53, 54, 55, 59, 63, 65 of SEQ ID NOs: 14, 59, 60, 61 or 62 or a combination thereof can be glutamic acid, aspartic acid, leucine, arginine, or asparagine. In some aspects, the amino acid substitution of the variant CDRH3 can be at positions 99, 100, 101, 102 of SEQ ID NOs: 14, 59, 60, 61 or 62 or a combination thereof. In some aspects, the amino acid substitution of the variant CDRH3 at positions 99, 100, 101, 102 of SEQ ID NOs: 14, 59, 60, 61 or 62 or a combination thereof can be tyrosine, aspartic acid, phenylalanine, glutamic acid.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are as described in Table 1, supra. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

In some aspects, the CDRs can be defined according to the Kabut definition. In some aspects, the CDRs can be defined according to the IMGT definition.

Disclosed herein are isolated antibody variants comprising a complementarily determining region light chain 1 (CDRL1) amino acid sequence of SEQ ID NOs: 38, 39, 40, 41, 42 or 43; a CDRL2 amino acid sequence of SEQ ID NOs: 44, 45, 46, 47, 48, 49, 50, or 51; and a CDRL3 amino acid sequence of SEQ ID NOs: 52, 53, 54 or 55. Also, disclosed herein are isolated antibody variants comprising a complementarity determining region heavy chain 1 (CDRH1) amino acid sequence of SEQ ID NOs: 16, 17, 18, 19, 20 or 21; a CDRH2 amino acid sequence of SEQ ID NOs: 22, 23, 24, 25, 26, 27, 28, 29, or 30; and a CDRH3 amino acid sequence of SEQ ID NOs: 31, 32, 33, 34, 35, 36 or 37. Disclosed herein are isolated antibody variants comprising a complementarity determining region light chain 1 (CDRL1) amino acid sequence of SEQ ID NOs: 38, 39, 40, 41, 42 or 43; a CDRL2 amino acid sequence of SEQ ID NOs: 44, 45, 46, 47, 48, 49, 50, or 51; and a CDRL3 amino acid sequence of SEQ ID NOs: 52, 53, 54 or 55; and a complementarity determining region heavy chain 1 (CDRH1) amino acid sequence of SEQ ID NOs: 16, 17, 18, 19, 20 or 21; a CDRH2 amino acid sequence of SEQ ID NOs: 22, 23, 24, 25, 26, 27, 28, 29, or 30; and a CDRH3 amino acid sequence of SEQ ID NOs: 31, 32, 33, 34, 35, 36 or 37.

TABLE 5
Exemplary Amino acid sequences of variant CDRs
of an anti-CCL21 antibody

| SEQ | | |
|---|---|---|
| ID NO: | Sequence | Description |
| 16 | EFYLN | CDRH1, Kabat |
| 17 | DFYIN | CDRH1, Kabat |
| 18 | EFYIN | CDRH1, Kabat |
| 19 | DFYLQ | CDRH1, Kabat |
| 20 | EFYLQ | CDRH1, Kabat |
| 21 | EFYIQ | CDRH1, Kabat |
| 22 | WIEPENGDTEYAPKFQG | CDRH2, Kabat |
| 23 | WIDPDNGDTEYAPKFQG | CDRH2, Kabat |
| 24 | WLDPENGDTEYAPKFQG | CDRH2, Kabat |
| 25 | WIDPEQGDTEYAPKFQG | CDRH2, Kabat |
| 26 | WIDPENGDTDYAPKFQG | CDRH2, Kabat |
| 27 | WEDPENGDTEYAPKFNG | CDRH2, Kabat |
| 28 | WIDPENGDTEYAPRFQG | CDRH2, Kabat |
| 29 | WIDPEQGDTEYAPKFQG | CDRH2, Kabat |
| 30 | WIEPDNGDTDYAPKFQG | CDRH2, Kabat |
| 31 | FEYDY | CDRH3, Kabat |
| 32 | FDYEY | CDRH3, Kabat |
| 33 | FDFDY | CDRH3, Kabat |
| 34 | FDYDF | CDRH3, Kabat |
| 35 | YDYDY | CDRH3, Kabat |
| 36 | FDFDY | CDRH3, Kabat |
| 37 | FEYEY | CDRH3, Kabat |
| 38 | HASQNIQVWLS | CDRL1, Kabat |
| 39 | HASQNINVWIS | CDRL1, Kabat |
| 40 | HASQNLNVWLS | CDRL1, Kabat |
| 41 | HVSQNINLWLS | CDRL1, Kabat |
| 42 | HASQNINVWLS | CDRL1, Kabat |
| 43 | WASQNINVWLS | CDRL1, Kabat |
| 44 | RASNLHT | CDRL2, Kabat |
| 45 | KLSNLHT | CDRL2, Kabat |
| 46 | KASNIHT | CDRL2, Kabat |
| 47 | KASQLHT | CDRL2, Kabat |
| 48 | KASNLWT | CDRL2, Kabat |
| 49 | KASNLHS | CDRL2, Kabat |
| 50 | KATNLHT | CDRL2, Kabat |
| 51 | RASQLHT | CDRL2, Kabat |
| 52 | HNGHSFPLT | CDRL3, Kabat |
| 53 | HQGHTFPLT | CDRL3, Kabat |
| 54 | HQGHSFPIT | CDRL3, Kabat |
| 55 | HQGHSFPLS | CDRL3, Kabat |

In some aspects, the antibodies disclosed herein can be recombinantly engineered, chimerized, or humanized. Table 5 shows examples of amino acid sequences of humanized heavy chain variable domains and humanized light chain variable domains of an anti-CCL21 antibody. In some aspects, the antibodies disclosed herein can be affinity matured or human antibodies. In some aspects, the antibodies disclosed herein can be a Fab, an Fab′, an F(ab′)2, a Fv, a scFv, a diabody or fragments thereof. In some aspects, the antibody can be a monoclonal antibody. In some aspects, the monoclonal antibodies can be humanized or chimeric forms thereof. In some aspects, the monoclonal antibody can be a humanized antibody. By known means and as described herein, polyclonal or monoclonal antibodies, antibody fragments, binding domains and CDRs (including engineered forms of any of the foregoing) may be created that are specific for CCL21 antigen, one or more of its respective epitopes, or conjugates of any of the foregoing, whether such antigens or epitopes are isolated from natural sources or are synthetic derivatives or variants of the natural protein.

TABLE 6
Exemplary Amino acid sequences of humanized heavy chain variable
domains, humanized light chain variable domains, and humanized
CDRs of an anti-CCL21 antibody

| SEQ ID NO: | Sequence | Description |
|---|---|---|
| 59 | EVQLVQSGAEVKKPGASVKVSCKASGF | Hu-VH1, IMGT |
| | NIKDFYLNWVRQAPGQGLEWIGWIDPE | |
| | NGDTEYAPKFQGRVTMTADTSSNTIYM | |
| | ELSSLRSEDTAVYYCNAFDYDYWGQGT | |
| | TVTVSS | |
| 60 | QVQLVQSGAEVKKPGASVKVSCKASGF | Hu-VH2, IMGT |
| | NIKDFYLNWVRQAPGQGLEWMGWIDPE | |
| | NGDTEYAPKFQGRVTMTADTSTNTIYM | |
| | ELSSLRSEDTAVYYCNAFDYDYWGQGT | |
| | TVTVSS | |
| 61 | EVQLVQSGSELKKPGASVKVSCKASGF | Hu-VH3, IMGT |
| | NIKDFYLNWVRQAPGQGLEWIGWIDPE | |
| | NGDTEYAPGFTGRFVMSADTSSNTIYL | |
| | QISSLKAEDTAVYYCNAFDYDYWGQGT | |
| | TVTVSS | |
| 62 | QVQLVQSGSELKKPGASVKVSCKASGF | Hu-VH4, IMGT |
| | NIKDFYLNWVRQAPGQGLEWMGWIDPE | |
| | NGDTEYAPGFTGRFVFSADTSVNTIYL | |
| | QISSLKAEDTAVYYCNAFDYDYWGQGT | |
| | TVTVSS | |
| 63 | DIQMTQSPSSLSASVGDRVTITCQASQNIN | Hu-VL1-v1, |
| | VWLSWYQQKPGKAPKLLIYKASNLHTGV | IMGT |
| | PSRFSGSGSGTDFTFTISSLQPEDIATYYCH | |
| | QGHSFPLTFGGGTKLEIK | |
| 64 | DIQMTQSPSSVSASVGDRVTITCRASQNIN | Hu-VL2-v2, |
| | VWLSWYQQKPGKAPKLLIYKASNLHTGV | IMGT |
| | PSRFSGSGSGTDFTLTISSLQPEDFATYYCH | |
| | QGHSFPLTFGGGTKLEIK | |
| 65 | DIQMTQSPSSVSASVGDRVTITCRASQNIN | Hu-VL3-v3, |
| | VWLSWYQQKPGKAPKLLIYKASNLHSGV | IMGT |
| | PSRFSGSGSGTDFTLTISSLQPEDFATYYCH | |
| | QGHSFPLTFGGGTKLEIK | |
| 66 | DIVMTQSPATLSLSPGERATLSCRASQNIN | Hu-VL4-v4, |
| | VWLSWYQQKPGQAPRLLIYKASNLHTGIP | IMGT |
| | ARFSGSGSGTDFTLTISSLEPEDFAVYYCH | |
| | QGHSFPLTFGGGTKLEIK | |

Disclosed herein are humanized antibodies comprising a light chain variable region amino acid sequence of SEQ ID NOs: 63, 64, 65 or 66. Disclosed herein are humanized antibodies comprising a heavy chain variable region amino acid sequence of SEQ ID NOs: 59, 60, 61 or 62. In some aspects, the humanized antibody can comprise a light chain variable region amino acid sequence of any of SEQ ID NOs: 63, 64, 65 or 66 and a heavy chain variable region amino acid sequence of SEQ ID NOs: 59, 60, 61 or 62. In some aspects, the humanized antibody can comprise a light chain variable region amino acid sequence of SEQ ID NO: 64 and a heavy chain variable region amino acid sequence of SEQ ID NO: 60.

A monoclonal antibody is a single, clonal species of antibody wherein every antibody molecule recognizes the same epitope because all antibody producing cells are derived from a single, antibody-producing B-lymphocyte (or other clonal cell, such as a cell that recombinantly expresses the antibody molecule). The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions. Hybridoma technology as used in monoclonal antibody production involves the fusion of a single, antibody-producing B lymphocyte isolated from a mouse previously immunized with a CCL21 protein or peptide with an immortalized cell, e.g., a mouse cell line. This technology provides a method to propagate a single antibody-producing cell for an indefinite number of generations, such that unlimited quantities of structurally identical antibodies having the same antigen or epitope specificity, i.e., monoclonal antibodies, may be produced.

Methods have been developed to replace light and heavy chain constant domains of the monoclonal antibody with analogous domains of human origin, leaving the variable regions of the foreign antibody intact. Alternatively, “fully human” monoclonal antibodies are produced in mice or rats that are transgenic for human immunoglobulin genes. Methods have also been developed to convert variable domains of monoclonal antibodies to more human form by recombinantly constructing antibody variable domains having both rodent and human amino acid sequences. In “humanized” monoclonal antibodies, only the hypervariable CDRs are derived from non-human (e.g., mouse, rat, chicken, llama, etc.) monoclonal antibodies, and the framework regions are derived from human antibody amino acid sequences. The replacement of amino acid sequences in the antibody that are characteristic of rodents with amino acid sequences found in the corresponding positions of human antibodies reduces the likelihood of adverse immune reaction to foreign protein during therapeutic use in humans. A hybridoma or other cell producing an antibody may also be subject to genetic mutation or other changes, which may or may not alter the binding specificity of antibodies produced by the hybridoma.

Engineered antibodies may be created using monoclonal and other antibodies and recombinant DNA technology to produce other antibodies or chimeric molecules that retain the antigen or epitope binding specificity of the original antibody, i.e., the molecule has a specific binding domain. Such techniques may involve introducing DNA encoding the immunoglobulin variable region or the CDRs of an antibody into the genetic material for the framework regions, constant regions, or constant regions plus framework regions, of a different antibody. See, for instance, U.S. Pat. Nos. 5,091,513 and 6,881,557, which are incorporated herein by reference.

By known means as described herein, polyclonal or monoclonal antibodies, antibody fragments having binding activity, binding domains and CDRs (including engineered forms of any of the foregoing), may be created that specifically bind to CCL21 protein, one or more of its respective epitopes, or conjugates of any of the foregoing, whether such antigens or epitopes are isolated from natural sources or are synthetic derivatives or variants of the natural compounds.

Antibodies may be produced from any animal source, including birds and mammals. In some aspects, the antibodies can be ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in Marks et al., 1992, Bio/Technol., 10:779-783; Stemmer, 1994, Nature, 370:389-391; Gram et al., 1992, Proc. Natl. Acad. Sci. USA, 89:3576-3580; Barbas et al., 1994, Proc. Natl. Acad. Sci. USA, 91:3809-3813; and Schier et al., 1996, Gene, 169(2):147-155.

Methods for producing polyclonal antibodies in various animal species, as well as for producing monoclonal antibodies of various types, including humanized, chimeric, and fully human, are well known in the art and are highly reproducible. For example, the following U.S. patents provide descriptions of such methods and are herein incorporated by reference: U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,420,253; 5,565,332; 5,571,698; 5,627,052; 5,656,434; 5,770,376; 5,789,208; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; 6,891,024; 7,407,659; and 8,178,098.

In some aspects, the antibody can be a single chain antibody. In some aspects, the antibody can be linked to a detectable label. In some aspects, antibody can be a monovalent or a bivalent antibody.

In some aspects, the antibodies disclosed herein can be an IgG, an IgM, an IgA, an IgD, or an IgE antibody or antigen binding fragment thereof. In some aspects, the antibodies can be of the IgG, IgM, IgA, IgD, and IgE Ig classes or a genetically modified IgG class antibody, as well as polypeptides comprising one or more antibody CDR regions that retain antigen binding activity. In some aspects, the antibody can be an IgG class of antibody. In some aspects, the IgG class antibody can be an IgG1, IgG2, IgG3, or IgG4 class antibody.

In some aspects, the antibody can be a bispecific antibody. Unifying two antigen binding sites of different specificity into a single construct, bispecific antibodies have the ability to bring together two discreet antigens with exquisite specificity and therefore have great potential as therapeutic agents. Bispecific antibodies were originally made by fusing two hybridomas, each capable of producing a different immunoglobulin. Bispecific antibodies can also be produced by joining two scFv antibody fragments while omitting the Fc portion present in full immunoglobulins. Each scFv unit in such constructs can contain one variable domain from each of the heavy (V_{H}) and light (V_{L}) antibody chains, joined with one another via a synthetic polypeptide linker, the latter often being genetically engineered so as to be minimally immunogenic while remaining maximally resistant to proteolysis. Respective scFv units may be joined by a number of known techniques, including incorporation of a short (usually less than 10 amino acids) polypeptide spacer bridging the two scFv units, thereby creating a bispecific single chain antibody. The resulting bispecific single chain antibody is therefore a species containing two V_{H}/V_{L} pairs of different specificity on a single polypeptide chain, in which the V_{H} and V_{L} domains in a respective scFv unit are separated by a polypeptide linker long enough to allow intramolecular association between these two domains, such that the so-formed scFv units are contiguously tethered to one another through a polypeptide spacer kept short enough to prevent unwanted association between, for example, the V_{H} domain of one scFv unit and the V_{L} of the other scFv unit.

Examples of antibody fragments suitable for use include, without limitation: (i) the Fab fragment, consisting of V_{L}, V_{H}, C_{L}, and C_{H1} domains; (ii) the “Fd” fragment consisting of the V_{H} and C_{H1} domains; (iii) the “Fv” fragment consisting of the V_{L} and V_{H} domains of a single antibody; (iv) the “dAb” fragment, which consists of a V_{H} domain; (v) isolated CDR regions; (vi) F(ab′)2 fragments, a bivalent fragment comprising two linked Fab fragments; (vii) single chain Fv molecules (“scFv”), in which a V_{H} domain and a V_{L} domain are linked by a peptide linker that allows the two domains to associate to form a binding domain; (viii) bi-specific single chain Fv dimers (see U.S. Pat. No. 5,091,513); and (ix) diabodies, multivalent, or multispecific fragments constructed by gene fusion (U.S. Patent Appln. Pub. No. 20050214860). Fv, scFv, or diabody molecules may be stabilized by the incorporation of disulfide bridges linking the V_{H} and V_{L} domains. Minibodies comprising a scFv joined to a C_{H3} domain (Hu et al., 1996, Cancer Res., 56:3055-3061) may also be useful. In addition, antibody-like binding peptidomimetics are also contemplated. “Antibody like binding peptidomimetics” (ABiPs), which are peptides that act as pared-down antibodies and have certain advantages of longer serum half-life as well as less cumbersome synthesis methods, have been reported by Liu et al., 2003, Cell Mol. Biol., 49:209-216.

Animals may be inoculated with an antigen, such as a CCL21 polypeptide or peptide to generate an immune response and produce antibodies specific for the CCL21 polypeptide. Frequently, an antigen is bound or conjugated to another molecule to enhance the immune response. As used herein, a conjugate can be any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

The antibodies described herein directed to CCL21 will have the ability to neutralize, block, inhibit, or counteract the effects of CCL21 binding to CCR7 regardless of the animal species, monoclonal cell line or other source of the antibody. Certain animal species may be less preferable for generating therapeutic antibodies because they may be more likely to cause an immune or allergic response due to activation of the complement system through the “Fc” portion of the antibody. However, whole antibodies may be enzymatically digested into the “Fc” (complement binding) fragment, and into peptide fragments having the binding domains or CDRs. Removal of the Fc portion reduces the likelihood that this antibody fragment will elicit an undesirable immunological response and, thus, antibodies without an Fc portion may be preferential for prophylactic or therapeutic treatments. As described above, antibodies may also be constructed so as to be chimeric, humanized, or partially or fully human, so as to reduce or eliminate potential adverse immunological effects resulting from administering to an animal an antibody that has been produced in, or has amino acid sequences from, another species.

In some aspects, the antibodies disclosed herein bind to human CCL21 and does not cross-react with mouse CCL21. In some aspects, the antibody binds to CCL21 with an affinity of greater than or equal to 42 nM. In some aspects, the antibody selectively binds to CCL21 and inhibits binding of CCL21 to CCR7. In some aspects, the antibody selectively binds to human CCL21 and inhibits binding of human CCL21 to human CCR7.

The term “specifically binds” (or “immunospecifically binds”) is not intended to indicate that an antibody binds exclusively to its intended target. Rather, an antibody “specifically binds” if its affinity for its intended target is about, for example, 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some aspects, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁹ M⁻¹, or about 10⁻¹⁰ M⁻¹ to about 10¹¹ M⁻¹. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the antibodies described herein comprise a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 8, SEQ ID NO: 9 or SEQ ID NO: 70, and SEQ ID NO: 10, respectively; and/or a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 1 or SEQ ID NO: 68, SEQ ID NO: 2, and SEQ ID NO: 3, respectively.

In some aspects, the antibodies disclosed herein can specifically bind to an epitope of CCL21. In some aspects, the epitope of CCL21 can be KYSQRKIPAKVVR (SEQ ID NO: 67).

In some aspects, the antibodies disclosed herein can prevent, inhibit or block CCL21 binding to CCR7. In some aspects, the antibodies disclosed herein can inhibit the binding of human CCL21 to human CCR7. In some aspects, the antibodies disclosed herein can prevent, inhibit or block T-cell chemotaxis, and/or block MHC-incompatible T-cell migration to lymph nodes. In some aspects, the antibodies disclosed herein can prevent, inhibit or block T-cell adherence to endothelium, prevent, inhibit or block T-cell migration to lymph nodes, and/or intestinal mucosa, and skin. In some aspects, the antibodies disclosed herein can prevent, inhibit or prevent, inhibit or dendritic cell adherence to endothelium, and/or prevent, inhibit or block dendritic cell migration to lymph nodes, intestinal mucosa and skin. In some aspects, the antibodies disclosed herein can prevent, inhibit or block tumor metastases generally and, in some aspects, specifically, for example, prevent, inhibit or block metastatic spread of cancers of the breast, colon, lymphatic system, pancreatic, lung, skin including melanoma, esophageal, head and neck, and stomach.

Antibody proteins may be recombinant, or synthesized in vitro. It is contemplated that in anti-CCL21 antibody-containing compositions as described herein can comprise between about 0.001 mg and about 10 mg of total antibody polypeptide per ml. Thus, the concentration of antibody protein in a composition can be about, at least about or at most about or equal to 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, at most about, or equal to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds CCL21.

Disclosed herein are compositions comprising any of the antibodies or isolated antibodies described herein. In some aspects, the compositions can further comprise at least one pharmaceutically acceptable carrier or diluent.

In some aspects, the compositions described herein can comprise a detectable label or reporter. An antibody or an immunological portion of an antibody that retains binding activity, can be chemically conjugated to, or recombinantly expressed as, a fusion protein with other proteins. For the purposes as described herein, all such fused proteins are included in the definition of antibodies or an immunological portion of an antibody. In some aspects, antibodies and antibody-like molecules generated against CCL21 or polypeptides that are linked to at least one agent to form an antibody conjugate or payload are encompassed. In order to increase the efficacy of antibody molecules as diagnostic or therapeutic agents, it is conventional to link or covalently bind or complex at least one desired molecule or moiety to the antibody. Such a linked molecule or moiety may be, but is not limited to, at least one effector, detectable label or reporter molecule. Effector molecules comprise molecules having a desired activity, e.g., cytotoxic activity. Non-limiting examples of effector molecules that may be attached to antibodies include toxins, therapeutic enzymes, antibiotics, radio-labeled nucleotides and the like. By contrast, a reporter molecule or detectable label is defined as any moiety that may be detected using an assay. Non-limiting examples of reporter molecules and detectable labels that can be conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin, and the like. Several methods are known in the art for attaching or conjugating an antibody to a conjugate molecule or moiety. Some attachment methods involve the use of a metal chelate complex, employing by way of nonlimiting example, an organic chelating agent such a diethylenetriaminepentaacetic acid anhydride (DTPA); ethylenetriaminetetraacetic acid; N-chloro-p-toluenesulfonamide; and/or tetrachloro-3-6α-diphenylglycouril-3 attached to the antibody. Antibodies, particularly the monoclonal antibodies as described herein, may also be reacted with an enzyme in the presence of a coupling agent such as glutaraldehyde or periodate. Conjugates with fluorescein markers are conventionally prepared in the presence of these coupling agents or by reaction with an isothiocyanate. In some aspects, an anti-CCL21 antibody as described herein, particularly a binding fragment thereof, may be coupled or linked to a compound or substance, such as polyethylene glycol (PEG), to increase its in vivo half-life in plasma, serum, or blood following administration.

In some aspects, the antibodies described herein can be specifically bind to their intended target. In some aspects, the antibodies described herein have no off site binding.

Methods

Disclosed herein are methods for treating an autoimmune disease in a subject. The methods can comprise administering to the subject a therapeutically effective amount of any of the isolated antibodies described herein or any of the compositions described herein. In some aspects, the autoimmune disease can be Crohn's disease, ulcerative colitis, inflammatory bowel disease, scleroderma, atopic dermatitis, vitiligo, or psoriasis. In some aspects, the autoimmune disease can be a T-cell mediated autoimmune disease.

Disclosed herein are methods of treating or preventing allograft transplant rejection in a subject. In some aspects, the methods can comprise administering to the subject (e.g., a transplant recipient) a therapeutically effective amount of any of the isolated antibodies described herein or any of the composition disclosed herein. In some aspects, the subject can be a transplant recipient. In some aspects, the allograft transplant rejection can be an acute rejection. In some aspects, the allograft transplant rejection can be a chronic rejection. In some aspects, the rejection can be a host versus graft reaction (HVGR) or a graft versus host reaction (GVHR).

In some aspects, the antibodies disclosed herein can be used to treat and/or prevent rejection in organ and/or tissue transplant recipients (i.e., treat and/or prevent allograft rejection). In some aspects, the antibodies disclosed herein can be used to treat and/or prevent host immunologic rejection of one or more allografts. Allografts can be rejected through either a cell-mediated or humoral immune reaction of the recipient against transplant (histocompatibility) antigens present on the membranes of the donor's cells. The strongest antigens are governed by a complex of genetic loci termed human leukocyte group A (HLA) antigens. Together with the ABO blood groups antigens, they are the chief transplantation antigens detectable in humans. In some aspects, the antibodies disclosed herein can be used in conjunction with transplant of a kidney, heart, lung, liver, pancreas, small intestine, large intestine, or skin to prevent or ameliorate a host versus graft reaction (HVGR) or a graft versus host reaction (GVHR). In some aspects, the antibodies disclosed herein can be used in conjunction with an allogeneic hematopoietic stem cell transplant.

Rejection following transplantation can generally be broken into three categories: hyperacute, occurring hours to days following transplantation; acute, occurring days to months following transplantation; and chronic, occurring months to years following transplantation.

Hyperacute rejection is caused mainly by the production of host antibodies that attack the graft tissue. In a hyperacute rejection reaction, antibodies are observed in the transplant vascular very soon after transplantation. Shortly thereafter, vascular clotting occurs, leading to ischemia, eventual necrosis and death. The graft infarction is unresponsive to known immunosuppressive therapies. Because HLA antigens can be identified in vitro, pre-transplant screening is used to significantly reduce hyperacute rejection. As a consequence of this screening, hyperacute rejection is relative uncommon today.

Acute rejection is thought to be mediated by the accumulation of antigen specific cells in the graft tissue. The T-cell-mediated immune reaction against these antigens (i.e., HVGR or GVHR) is the principle mechanism of acute rejection. Accumulation of these cells leads to damage of the graft tissue. It is believed that both CD4+ helper T-cells and CD8+ cytotoxic T-cells are involved in the process, and that the antigen is presented by donor and host dendritic cells. The CD4+ helper T-cells help recruit other effector cells, such as macrophages and eosinophils, to the graft. Accessing T-cell activation signal transduction cascades (for example, CD28, CD40L and CD2 cascades) are also involved.

The cell-mediated acute rejection may be reversed in many cases by intensifying immunotherapy. After successful reversal, severely damaged elements of the graft heal by fibrosis and the remainder of the graft appears normal. After resolution of acute rejection, dosages of immunosuppressive drugs can be reduced to very low levels.

Chronic rejection, which is a particular problem in renal transplants, often progresses insidiously despite increased immunosuppressive therapy. It is thought to be due, in large part, to cell-mediated Type IV hypersensitivity. The pathologic profile differs from that of acute rejection. The arterial endothelium is primarily involved, with extensive proliferation that may gradually occlude the vessel lumen, leading to ischemia, fibrosis, a thickened intima and atherosclerotic changes. Chronic rejection is mainly due to a progressive obliteration of graft vasculature, and resembles a slow, vasculitic process.

Disclosed herein are methods of inducing immunosuppression or reducing immune system over activation or reducing immune system activation associated with CC chemokine ligand 21 (CCL21) levels in a subject. In some aspects, the CCL21 levels can be increased compared to a reference sample or control subject. In some aspects, the method comprises administering therapeutically effective amount of any of the isolated antibodies disclosed herein or any of the compositions disclosed herein. In some aspects, the method comprises administering therapeutically effective amount of any of the anti-CCL21 antibodies disclosed herein. In some aspects, the therapeutically effective amount of the anti-CCL21 antibodies can be an amount to form a complex between the anti-CCL21 antibody and CCL21 in the subject, thereby inducing immunosuppression or reducing immune system over activation or reducing immune system activation associated with CCL21 levels in the subject. In some aspects, the subject can suffer from a cancer, an autoimmune disease or an allograft rejection.

Further disclosed herein are methods of treating or preventing a relapse associated with an autoimmune disease in a subject. In some aspects, the methods can comprise administering to the subject (e.g., a patient in remission for an autoimmune disease) a therapeutically effective amount of any of the isolated antibodies described herein or any of the composition disclosed herein. In some aspects, the subject can be in a remission stage of an autoimmune disease. The remission stage of the autoimmune disease can mean that the autoimmune disease is no longer active. In some aspects, the autoimmune disease can be Crohn's disease, ulcerative colitis, inflammatory bowel disease, scleroderma, atopic dermatitis, vitiligo, or psoriasis. In some aspects, the autoimmune disease can be a T-cell mediated autoimmune disease. In some aspects, the subject can be a transplant recipient, wherein the subject was diagnosed with graft versus host disease, and the graft versus host disease is in remission.

Also disclosed herein are methods of treating or reducing metastatic cancer in a subject or preventing metastasis in a subject having cancer at risk for metastasis. Also disclosed herein are methods of preventing reoccurrence of a tumor in a subject. In some aspects, the method comprises administering therapeutically effective amount of any of the isolated antibodies disclosed herein or any of the compositions disclosed herein. In some aspects, the cancer can be a cancer of breast, colon, lymphatic system, pancreas, lung, skin (including melanoma), esophagus, head and neck, or stomach. In some aspects, the subject has cancer. In some aspects, the subject has metastatic cancer. In some aspects, the subject can have cancer or be a cancer patient and is at risk for cancer metastasis. In some aspects, administration of any of the antibodies disclosed herein can reduce the number of metastases. In some aspects, administration of any of the antibodies disclosed herein can prevent the occurrence or reoccurrence of metastasis. In some aspects, administration of any of the antibodies disclosed herein can increase the subject's or patient's survival time. In some aspects, administration of any of the antibodies disclosed herein can prevent the reoccurrence of a tumor in the subject.

In some aspects, the subject can be identified in need of treatment before the administering step. In some aspects, the antibody can be administered in a pharmaceutically acceptable composition. In some aspects, the antibody can be administered systemically, intravenously, intradermally, intramuscularly, intraperitoneally, subcutaneously or locally into inflamed tissues, organs or tumors.

In some aspects, the methods can further comprising administering one or more drugs or therapeutic agents to the subject. Examples of drugs or therapeutic agents that can be administered in combination with any of the antibodies described herein include but are not limited to prednisone, mesalamine, budesonide (Entocort EC), sulfasalazine (Azulfidine®), mesalamine (Asacol, Asacol HD, Lialda, Pentasa®, Apriso), azathioprine (Imuran), sulfazine, Remicade® (infliximab), dexamethasone, mercaptopurine, Acthar®, cyclosporine, tacrolimus, rapamycin, mycophenolate mofetil, rituximab, obinutuzumab, fedratinib, ruxolitinib, idelalisib, alpelisib, duvelisib, copanlisib, ibrutinib, zanubrutinib, or acalabrutinib.

Examples of drugs or therapeutic agents that can be administered in combination with any of the antibodies described herein, and in some aspects, to transplant recipient subjects, include but are not limited to cyclosporine, tacrolimus, sirolimus, an inhibitor of IMPDH, mycophenolate, mycophenolate mofetil, an anti-T-Cell antibody and OKT3.

Treatment of diseases. Disclosed herein are antibodies or antigen binding fragments thereof, as described herein (e.g., an antibody that specifically and preferentially binds to CCL21 and blocks or inhibits binding of CCL21 to CCR7) that can be used in treatment methods and administered to treat or prevent an autoimmune disease or disorder, allograft transplant rejection or metastatic cancer. Accordingly, provided herein are methods of treating an autoimmune disease, treating or preventing allograft transplant rejection, and treating metastatic cancer or preventing metastasis in a subject having cancer at risk for metastasis. In some aspects, the methods can comprise administering to a subject a therapeutically effective amount of any of the antibodies described herein or any of the compositions comprising at least one of antibodies as described herein. In some aspects, the drug or therapeutic agent can be an anti-CCL21 antibody or a composition comprising at least one anti-CCL21 antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with an autoimmune disease or cancer, or undergoing or have undergone an allograft transplant in an amount sufficient to at least partially improve a sign or symptom or to inhibit the progression of (and preferably arrest) the symptoms of the disease or condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the autoimmune disease or cancer or allograft rejection is delayed, hindered, or prevented, or the autoimmune disease or cancer or allograft rejection or a symptom of the autoimmune disease or disorder or allograft rejection is ameliorated or its frequency can be reduced. One or more of the symptoms can be less severe. Recovery can be accelerated in an individual who has been treated. For example, treatment of metastatic cancer may involve, for example, a reduction in the size of a tumor, a reduction in the invasiveness of a tumor, reduction in the growth rate of the cancer, or prevention of metastasis. Treatment of metastatic cancer may also refer to prolonging survival of a subject with cancer. In some aspects, the antibodies described herein can prolong the lifespan of a subject with cancer. In some aspects, the antibodies described herein can reduce or inhibit tumor cell growth.

Treatment of these subjects with an effective amount of at least one of the anti-CCL21 antibodies as described herein can result in binding of one or more of the disclosed antibodies to CCL21, thereby preventing, blocking or inhibiting CCL21 from binding to its cognate receptor, CCR7, and thereby preventing or avoiding immune system (over) activity or activation of T-cells (or inducing suppression of T-cell activity). Accordingly, the methods as provided are advantageous for a subject who is in need of, capable of benefiting from, or who is desirous of receiving the benefit of, the anti-cancer results, the amelioration of one or more autoimmune symptoms, or the anti-tumor rejection results achieved by the practice of the present methods. A subject's seeking the therapeutic benefits of the methods involving administration of at least one anti-CCL21 antibody in a therapeutically effective amount, or receiving such therapeutic benefits offer advantages to the art. In addition, the present methods offer the further advantages of eliminating or avoiding side effects, adverse outcomes, contraindications, and the like, or reducing the risk or potential for such issues to occur compared with other treatments and treatment modalities.

Autoimmune diseases for which the present methods are useful include but are not limited to Crohn's disease, ulcerative colitis, inflammatory bowel disease, scleroderma, atopic dermatitis, vitiligo, or psoriasis.

Cancers for which the present methods are useful include but are not limited to breast cancer, colon cancer, lymphatic system cancers, pancreatic cancer, lung cancer, skin cancers (including melanoma), esophageal cancer, head and neck cancer and stomach cancer.

Allograft rejections for which the present methods are useful include but are not limited to acute rejection, chronic rejection, host versus graft reaction, graft versus host reaction and allogeneic hematopoietic stem cell transplant. In some aspects, the present methods are useful in conjunction with transplants of a kidney, heart, lung, liver, pancreas, small intestine, large intestine, or skin.

The anti-CCL21 antibodies, such as monoclonal antibodies, can be used as immunosuppressant agents in a variety of modalities. In some aspects, the methods described herein use the antibodies disclosed herein as immunosuppressant agents, and, thus, comprise contacting a population of cells with a therapeutically effective amount of one or more of the antibodies, or a composition containing one or more of the antibodies, for a time period sufficient to block or inhibit one or more of the following: T-cell chemotaxis, MHC-incompatible T-cell migration to lymph nodes, T-cell adherence to endothelium, T-cell migration to lymph nodes, intestinal mucosa, and skin, dendritic cell adherence to endothelium, dendritic cell migration to lymph nodes, and/or intestinal mucosa and skin. In some aspects, contacting a cell in vivo is accomplished by administering to a subject in need, for example, by intravenous, subcutaneous, intraperitoneal, or intratumoral injection, a therapeutically effective amount of a physiologically tolerable composition comprising an anti-CCL21 antibody as described herein. The antibody may be administered parenterally by injection or by gradual infusion over time. Useful administration and delivery regimens include intravenous, intraperitoneal, oral, intramuscular, subcutaneous, intracavity, transdermal, dermal, peristaltic means, or direct injection into the tissue containing the cells.

Therapeutic compositions comprising antibodies are conventionally administered intravenously, such as by injection of a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required diluent, i.e., carrier, or vehicle. The compositions comprising any of the anti-CCL21 antibodies disclosed herein can be administered in a manner compatible with the dosage formulation, and in a therapeutically effective amount. The quantity to be administered depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimens for initial and booster administration are also contemplated and may typically involve an initial administration followed by repeated doses at one or more intervals (hours) by a subsequent injection or other administration. In some aspects, multiple administrations can be suitable for maintaining continuously high serum and tissue levels of antibody. Alternatively, continuous intravenous infusion sufficient to maintain concentrations in the blood in the ranges specified for in vivo therapies are contemplated.

It is contemplated that an anti-CCL antibody as described herein can be administered systemically or locally to treat disease, such as to inhibit tumor cell growth or to kill cancer cells in cancer patients with locally advanced or metastatic cancers or at risk for metastatic cancers. The antibodies can be administered alone or in combination with anti-proliferative drugs or anticancer drugs. In some aspects, the anti-CCL21 antibodies can be administered to reduce the cancer load in the patient prior to surgery or other procedures. Alternatively, they can be administered at periodic intervals after surgery to ensure that any remaining cancer (e.g., cancer that the surgery failed to eliminate) is reduced in size or growth capacity and/or does not survive. As noted herein, a therapeutically effective amount of an antibody can be a predetermined amount calculated to achieve the desired effect. Thus, the dosage ranges for the administration of an anti-CCL21 antibody are those large enough to produce the desired effect in which the symptoms of tumor cell division and cell cycling are reduced. Optimally, the dosage should not be so large as to cause adverse side effects, such as hyperviscosity syndromes, pulmonary edema, congestive heart failure, neurological effects, and the like. Generally, the dosage will vary with age of, condition of, size and gender of, and extent of the disease in the subject or patient and can be determined by one of skill in the art such as a medical practitioner or clinician. Of course, the dosage may be adjusted by the individual physician in the event of any complication.

It is also contemplated that an anti-CCL antibody as described herein can be administered systemically or locally to treat an autoimmune disease or allograft transplant rejection, such as to block, inhibit or prevent CCL21 binding to CCR7, block, inhibit or prevent T-cell chemotaxis, block, inhibit or prevent MHC-incompatible T-cell migration to lymph nodes, block, inhibit or prevent T-cell adherence to endothelium, block, inhibit or prevent T-cell migration to lymph nodes, intestinal mucosa, and skin, block, inhibit or prevent dendritic cell adherence to endothelium, and block, inhibit or prevent dendritic cell migration to lymph nodes, intestinal mucosa and skin patients with autoimmune diseases and/or transplant recipients. The antibodies can be administered alone or in combination with other drugs or therapeutic agents.

In some aspects, the anti-CCL21 antibodies can be administered to induce immunosuppression in the patient prior to surgery or allograft transplantation or other procedures. Alternatively, they can be administered at periodic intervals after surgery or transplantation to facilitate allograft survival. As noted herein, a therapeutically effective amount of an antibody can be a predetermined amount calculated to achieve the desired effect. Thus, the dosage ranges for the administration of an anti-CCL21 antibody are those large enough to produce the desired effect in which one or more symptoms of an autoimmune disease and allograft rejection are reduced. Optimally, the dosage should not be so large as to cause adverse side effects. Generally, the dosage will vary with age of, condition of, size and gender of, and extent of the disease or symptoms in the subject or patient and can be determined by one of skill in the art such as a medical practitioner or clinician. Of course, the dosage may be adjusted by the individual physician in the event of any complication.

Treatment methods. In some aspects, the compositions and methods as described herein comprise the administration of an anti-CCL21 antibody as described herein, alone, or in combination with a second or additional drug or therapy. Such drug or therapy may be applied in the treatment of any disease that is associated with CCL21, and in some aspects, the interaction of human CCL21 or with human CCR7. For example, the disease can be an autoimmune disease, cancer, metastatic cancer or allograft transplant rejection. The compositions and methods described herein can comprise at least one anti-CCL21 antibody that preferentially binds to CCR7 protein and has a therapeutic or protective effect in the treatment of an autoimmune disease, metastatic cancer or transplant rejection, particularly by preventing, reducing, blocking, or inhibiting the CCR7/CCL21 interaction, thereby providing a therapeutic effect and treatment.

The compositions and methods, including combination therapies, have a therapeutic or protective effect and may enhance the therapeutic or protective effect, and/or increase the therapeutic effect of another drug, therapy or therapeutic agent (e.g., anti-cancer or anti-hyperproliferative therapy).

Therapeutic and prophylactic methods and compositions can be provided in a combined amount effective to achieve the desired effect, such as the killing of a cancer cell and/or the inhibition of cellular hyperproliferation; reducing one or more symptoms of an autoimmune disease, reducing CCL21 levels, preventing allograft transplant rejection). This process may involve administering an anti-CCL21 antibody or a binding fragment thereof and a second therapy. The second therapy may or may not have a direct cytotoxic effect. A tissue, tumor, and/or cell can be exposed to one or more compositions or pharmacological formulation(s) comprising one or more of the agents (e.g., an antibody or an anti-cancer agent), or by exposing the tissue, tumor, and/or cell with two or more distinct compositions or formulations, wherein one composition provides, for example, 1) an antibody, 2) an anti-cancer agent, 3) both an antibody and an anti-cancer agent, or 4) two or more antibodies. In some aspects, the second therapy can be also an anti-CCL21 antibody. Also, it is contemplated that such a combination therapy can be used in conjunction with chemotherapy, radiotherapy, surgical therapy, or immunotherapy.

By way of example, the terms “contacted” and “exposed,” when applied to a cell, are used herein to describe a process by which a therapeutic polypeptide, for example, an anti-CCL21 antibody as described herein, is delivered to a target cell or is placed in direct juxtaposition with the target cell, particularly to bind specifically to the target antigen, e.g., CCL21, expressed or highly expressed on the surface of endothelial venules, T-cell zones in lymph nodes and other secondary lymphoid organs. Such binding by a therapeutic anti-CCL21 antibody or binding fragment thereof prevents, blocks, inhibits, or reduces the interaction of CCL21 with CCR7 on an effector T-cell, thereby preventing immune system activation associated with the CCL21/CCR7 interaction. In some aspects, a chemotherapeutic or radiotherapeutic agent can also be administered or delivered to the subject in conjunction with the anti-CCL21 antibody or binding fragment thereof. To achieve cell killing, for example, one or more agents are delivered to a cell in a combined amount effective to kill the cell or prevent it from dividing.

Any of the anti-CCL21 antibodies disclosed herein may be administered before, during, after, or in various combinations relative to another treatment (e.g., anti-cancer, or immunosuppressant agent). The administrations may be in intervals ranging from concurrently to minutes to days to weeks before or after one another. In some aspects, in which the antibody is provided to a patient separately from an anti-cancer agent or immunosuppressant agent, it would be generally ensured that a significant period of time did not expire between the time of each delivery, such that the administered compounds would still be able to exert an advantageously combined effect for the patient. Illustratively, in such instances, it is contemplated that one may provide a patient with the antibody and the anti-cancer therapy or immunosuppressant agent within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment or treatment cycle will last 1-90 days or more (this range includes intervening days and the last day). It is contemplated that one agent may be given on any day of day 1 to day 90 (this such range includes intervening days and the last day) or any combination thereof, and another agent is given on any day of day 1 to day 90 (this such range includes intervening days and the last day) or any combination thereof Within a single day (24-hour period), the patient may be given one or multiple administrations of the agent(s). Moreover, after a course of treatment, it is contemplated that there may be a period of time at which no second agent (e.g., anti-cancer treatment or immunosuppressant agent) is administered. This time period may last, for example, for 1-7 days, and/or 1-5 weeks, and/or 1-12 months or more (this such range includes intervening days and the upper time point), depending on the condition of the patient, such as prognosis, strength, health, etc. Treatment cycles would be repeated as necessary. Various combinations of treatments may be employed. In the representative examples of combination treatment regimens shown below, an antibody, such as an anti-CCL21 antibody or binding fragment thereof is represented by “A” and an anti-cancer therapy is represented by “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A.

Administration of any antibody or therapy as described herein to a patient will follow general protocols for the administration of such compounds, taking into account the toxicity, if any, of the agents. Therefore, in some aspects there is a step of monitoring adverse events and toxicity, particularly those that may be attributable to combination therapy.

In some aspects, methods are disclosed comprising administering an anti-CCL21 antibody alone or in combination with another agent (e.g., anticancer agent or immunosuppressant agent) to a subject in need thereof, i.e., a subject with a cancer or a tumor, an autoimmune disease, or an allograft transplant recipient). Prior to administration of the anti-CCL21 antibody, a sample of the subject's tumor or cancer or one or more symptoms associated with the autoimmune disease or rejection of the allograft may be evaluated for the presence or level of CCL21. If the results of such an evaluation reveals that the subject's tumor or cancer or symptoms associated with the autoimmune disease or rejection of the allograft is positive for CCL21 or the level of CCL21 is increased compared to a reference sample or prior sample from the same subject, the subject would be selected for treatment based on the likelihood that subject's CCL21+ tumor or cancer or disease state or condition would be more amenable to treatment with the anti-CCL21 antibody and treatment may proceed with a more likely beneficial outcome. A medical professional or physician may advise the subject to proceed with the anti-CCL21 antibody treatment method, and the subject may decide to proceed with treatment based on the advice of the medical professional or physician. In addition, during the course of treatment, the subject's tumor or cancer cells or blood cells may be assayed for the presence of CCL21 as a way to monitor the progress or effectiveness of treatment. If the assay shows a change, loss, or decrease, for example, in CCL21 on the subject's tumor or cancer cells or blood cells, a decision may be taken by the medical professional in conjunction with the subject as to whether the treatment should continue or be altered in some fashion, e.g., a higher dosage, the addition of another anti-cancer agent or therapy or immunosuppressant, and the like.

Chemotherapy. A wide variety of chemotherapeutic agents may be used in accordance with the treatment or therapeutic methods as described herein. The term “chemotherapy” refers to the use of drugs to treat cancer. A “chemotherapeutic agent” connotes a compound or composition that is administered in the treatment of cancer. Such agents or drugs are categorized by their mode of activity within a cell, for example, whether and at what stage they affect the cell cycle and cell growth and proliferation. Alternatively, a chemotherapeutic agent may be characterized based on its ability to directly cross-link DNA, to intercalate into DNA, or to induce chromosomal and mitotic aberrations by affecting nucleic acid synthesis in a cell.

Nonlimiting examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabine, navelbine, farnesyl-protein transferase inhibitors, transplatinum, and pharmaceutically acceptable salts, acids, or derivatives of any of the above.

Radiotherapy. Radiotherapy includes treatments with agents that cause DNA damage. Radiotherapy has been used extensively in cancer and disease treatments and embraces what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA itself, on the precursors of DNA, on the replication and repair of DNA, and on the assembly and maintenance of chromosomes. Exemplary dosage ranges for X-rays range from daily doses of 50 to 200 roentgens for prolonged periods of time (3 to 4 weeks) to single doses of 2000 to 6000 roentgens. Dosage ranges for radioisotopes vary widely and depend on the half-life of the isotope, the strength and type of radiation emitted, the uptake by the neoplastic cells, and tolerance of the subject undergoing treatment.

Immunotherapy. In some aspects of the methods, immunotherapies may be used in combination or in conjunction with administration of anti-CCL21 antibodies as described herein. In the context of cancer treatment, immunotherapeutics generally rely on the use of immune effector cells and molecules to target and destroy cancer cells. Rituximab (RITUXAN®) is such an example. In some aspects, checkpoint inhibitors can also be administered in combination, including ipilimumab. The anti-CCL21 antibodies can be administered in combination with anti-PD-1 or anti-PD-L1 inhibitors, such as antibodies against PD-L1, which include atezolizumab, durvalumab, or avelumab, or antibodies against PD-1, including nivolumab, pembrolizumab, or pidilizumab. In addition, one or more of the anti-CCL21 antibodies as described herein may be administered in combination with each other. In some aspects, the anti-CCL21 antibodies can also be administered in combination with CCL21 inhibitors. The antibody alone may serve as an effector of therapy or it may recruit other cells to actually affect cell killing or suppress the immune system. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target, e.g., the PD-1 on T-cells/PD-L1 on tumor cells interaction. Various effector cells include cytotoxic T cells and natural killer (NK) cells.

In the context of treating an autoimmune disease or disorder or treating an allograft transplant recipient, one or more of the immunotherapeutics can be administered to the subject.

In some aspects of immunotherapy, the tumor cell must bear some marker (protein/receptor) that is amenable to targeting. Optimally, the tumor marker protein/receptor is not present on the majority of other cells, such as non-cancer cells or normal cells. Many tumor markers exist and any of these may be suitable for targeting by another drug or therapy administered with an anti-CCL21 antibody as disclosed herein. Common tumor markers include, for example, CD20, carcinoembryonic antigen (CEA), tyrosinase (p97), gp68, TAG-72, HMFG, Sialyl Lewis Antigen, MucA, MucB, PLAP, laminin receptor, erbB, and p155. An alternative aspect of immunotherapy is to combine anticancer effects with immune stimulatory effects. Immune stimulating molecules also exist and include cytokines, such as IL-2, IL-4, IL-12, GM-CSF, gamma-IFN; chemokines, such as MIP-1, MCP-1, IL-8; and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui et al., 1998, Infection Immun., 66(11):5329-5336; Christodoulides et al., 1998, Microbiology, 144(Pt 11):3027-3037); cytokine therapy, e.g., α, β, and γ interferons; IL-1, GM-CSF, and TNF (Bukowski et al., 1998, Clinical Cancer Res., 4(10):2337-2347; Davidson et al., 1998, J. Immunother., 21(5):389-398; Hellstrand et al., 1998, Acta Oncologica, 37(4):347-353); gene therapy, e.g., TNF, IL-1, IL-2, and p53 (Qin et al., 1998, Proc. Natl. Acad. Sci. USA, 95(24):14411-14416; Austin-Ward and Villaseca, 1998, Revista Medica de Chile, 126(7):838-845; U.S. Pat. Nos. 5,830,880 and 5,846,945); and monoclonal antibodies, e.g., anti-CD20, anti-ganglioside GM2, and anti-p185 (Hollander, 2012, Front. Immun., 3:3; Hanibuchi et al., 1998, Int. J. Cancer, 78(4):480-485; U.S. Pat. No. 5,824,311). It is contemplated that one or more anti-cancer therapies may be employed with the antibody therapies described herein.

Surgery. Approximately 60% of individuals with cancer undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as anti-CCL21 antibody treatment as described herein, chemotherapy, radiotherapy, hormonal therapy, gene therapy, immunotherapy, and/or alternative therapies, as well as combinations thereof. Tumor resection refers to physical removal of at least part of a tumor. In addition to tumor resection, treatment by surgery includes laser surgery, cryosurgery, electrosurgery, and microscopically-controlled surgery (Mohs' surgery). Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 6, or 7 days, or every 1, 2, 3, 4, and 5 weeks or every 1, 2, 3, 4, 5, 6, 7, 8, 9, 10, 11, or 12 months. These treatments may be of varying dosages as well.

Protein Purification. Protein, including antibody and, particularly, anti-CCL21 antibody, purification techniques are well known to those of skill in the art. These techniques involve, at one level, the homogenization and crude fractionation of the cells, tissue, or organ into polypeptide and non-polypeptide fractions. The protein or polypeptide of interest may be further purified using chromatographic and electrophoretic techniques to achieve partial or complete purification (or purification to homogeneity) unless otherwise specified. Analytical methods particularly suited to the preparation of a pure protein or peptide are ion-exchange chromatography, size-exclusion chromatography, reverse phase chromatography, hydroxyapatite chromatography, polyacrylamide gel electrophoresis, affinity chromatography, immunoaffinity chromatography, and isoelectric focusing. A particularly efficient method of purifying peptides is fast-performance liquid chromatography (FPLC) or even high-performance liquid chromatography (HPLC). As is generally known in the art, the order of conducting the various purification steps may be changed, and/or certain steps may be omitted, and still result in a suitable method for the preparation of a substantially purified polypeptide.

A purified polypeptide, such as an anti-CCL21 antibody as described herein, refers to a polypeptide which is isolatable or isolated from other components and purified to any degree relative to its naturally-obtainable state. An isolated or purified polypeptide, therefore, also refers to a polypeptide free from the environment in which it may naturally occur, e.g., cells, tissues, organs, biological samples, and the like. Generally, “purified” will refer to a polypeptide composition that has been subjected to fractionation to remove various other components, and which composition substantially retains its expressed biological activity. A “substantially purified” composition refers to one in which the polypeptide forms the major component of the composition, and as such, constitutes about 50%, about 60%, about 70%, about 80%, about 90%, about 95%, or more of the protein component of the composition.

Various methods for quantifying the degree of purification of polypeptides, such as antibody proteins, are known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity therein, assessed by a “fold purification number.” The actual units used to represent the amount of activity will, of course, be dependent upon the particular assay technique chosen to follow the purification, and whether or not the expressed polypeptide exhibits a detectable activity.

There is no general requirement that the polypeptide will always be provided in its most purified state. Indeed, it is contemplated that less substantially purified products may have utility in some aspects. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “fold” purification than the same technique utilizing a low pressure chromatography system. Methods exhibiting a lower degree of relative purification may have advantages in total recovery of protein product, or in maintaining the activity of an expressed protein.

Affinity chromatography is a chromatographic procedure that relies on the specific affinity between a substance (protein) to be isolated and a molecule to which it can specifically bind, e.g., a receptor-ligand type of interaction. The column material (resin) is synthesized by covalently coupling one of the binding partners to an insoluble matrix. The column material is then able to specifically adsorb the substance from the solution that is passed over the column resin. Elution occurs by changing the conditions to those in which binding will be disrupted/will not occur (e.g., altered pH, ionic strength, temperature, etc.). The matrix should be a substance that does not adsorb molecules to any significant extent and that has a broad range of chemical, physical, and thermal stability. The ligand should be coupled in such a way as to not affect its binding properties. The ligand should also provide relatively tight binding; however, elution of the bound substance should occur without destroying the sample protein desired or the ligand.

Size-exclusion chromatography (SEC) is a chromatographic method in which molecules in solution are separated based on their size, or in more technical terms, their hydrodynamic volume. It is usually applied to large molecules or macromolecular complexes, such as proteins and industrial polymers. Typically, when an aqueous solution is used to transport the sample through the column, the technique is known as gel filtration chromatography, versus the name gel permeation chromatography, which is used when an organic solvent is used as a mobile phase. The underlying principle of SEC is that particles of different sizes will elute (filter) through a stationary phase at different rates, resulting in the separation of a solution of particles based on size. Provided that all of the particles are loaded simultaneously or near simultaneously, particles of the same size should elute together.

High-performance (aka high-pressure) liquid chromatography (HPLC) is a form of column chromatography used frequently in biochemistry and analytical chemistry to separate, identify, and quantify compounds. HPLC utilizes a column that holds chromatographic packing material (stationary phase), a pump that moves the mobile phase(s) through the column, and a detector that shows the retention times of the molecules. Retention time varies depending on the interactions between the stationary phase, the molecules being analyzed, and the solvent(s) used

Pharmaceutical Preparations. Where clinical application of a pharmaceutical composition comprising an anti-CCL21 antibody is undertaken, it is generally beneficial to prepare a pharmaceutical or therapeutic composition appropriate for the intended application. In general, pharmaceutical compositions can comprise an effective amount of one or more polypeptides or additional agents dissolved or dispersed in a pharmaceutically acceptable carrier. In some aspects, pharmaceutical compositions may comprise, for example, at least about 0.1% of a polypeptide or antibody. In some aspects, a polypeptide or antibody may comprise between about 2% to about 75% of the weight of the unit, or between about 25% to about 60%, for example, and any range derivable there between, including the upper and lower values. The amount of active compound(s) in each therapeutically useful composition may be prepared in such a way that a suitable dosage will be obtained in any given unit dose. Factors, such as solubility, bioavailability, biological half-life, route of administration, product shelf life, as well as other pharmacological considerations, are contemplated by one skilled in the art of preparing such pharmaceutical formulations, and as such, a variety of dosages and treatment regimens may be desirable.

Further in some aspects, the composition suitable for administration can be provided in a pharmaceutically acceptable carrier with or without an inert diluent. The carrier should be assimilable and include liquid, semi-solid, e.g., gels or pastes, or solid carriers. Examples of carriers or diluents include but are not limited to fats, oils, water, saline solutions, lipids, liposomes, resins, binders, fillers, and the like, or combinations thereof. As used herein, “pharmaceutically acceptable carrier” includes any and all aqueous solvents (e.g., water, alcoholic/aqueous solutions, ethanol, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings (e.g., lecithin), surfactants, antioxidants, preservatives (e.g., antibacterial or antifungal agents, anti-oxidants, chelating agents, inert gases, parabens (e.g., methylparabens, propylparabens), chlorobutanol, phenol, sorbic acid, thimerosal), isotonic agents (e.g., sugars, sodium chloride), absorption delaying agents (e.g., aluminum monostearate, gelatin), salts, drugs, drug stabilizers (e.g., buffers, amino acids, such as glycine and lysine, carbohydrates, such as dextrose, mannose, galactose, fructose, lactose, sucrose, maltose, sorbitol, mannitol, etc.), gels, binders, excipients, disintegration agents, lubricants, sweetening agents, flavoring agents, dyes, fluid and nutrient replenishers, such like materials and combinations thereof, as would be known to one of ordinary skill in the art. Except insofar as any conventional media, agent, diluent, or carrier is detrimental to the recipient or to the therapeutic effectiveness of the composition contained therein, its use in an administrable composition for the practice of the methods is appropriate. The pH and exact concentration of the various components in a pharmaceutical composition are adjusted according to well-known parameters. In some aspects, the composition can be combined with the carrier in any convenient and practical manner, i.e., by solution, suspension, emulsification, admixture, encapsulation, absorption, grinding, and the like. Such procedures are routine for those skilled in the art.

In some aspects, the compositions may comprise different types of carriers depending on whether they are to be administered in solid, liquid, or aerosol form, and whether it needs to be sterile for the route of administration, such as injection. The compositions can be formulated for administration intravenously, intradermally, transdermally, intrathecally, intra-arterially, intraperitoneally, intranasally, intravaginally, intrarectally, intramuscularly, subcutaneously, mucosally, orally, topically, locally, by inhalation (e.g., aerosol inhalation), by injection, by infusion, by continuous infusion, by localized perfusion bathing target cells directly, via a catheter, via a lavage, in lipid compositions (e.g., liposomes), or by other methods or any combination of the forgoing as would be known to one of ordinary skill in the art. See, for example, Remington's Pharmaceutical Sciences, 18th Ed., 1990. Typically, such compositions can be prepared as either liquid solutions or suspensions; solid or reconstitutable forms suitable for use to prepare solutions or suspensions upon the addition of a liquid prior to injection can also be prepared; and, the preparations can also be emulsified.

The antibodies may be formulated into a composition in a free base, neutral, or salt form. Pharmaceutically acceptable salts include the acid addition salts, e.g., those formed with the free amino groups of a proteinaceous composition, or which are formed with inorganic acids, such as, for example, hydrochloric or phosphoric acids, or such organic acids as acetic, oxalic, tartaric, or mandelic acid. Salts formed with the free carboxyl groups may also be derived from inorganic bases, such as, for example, sodium, potassium, ammonium, calcium, or ferric hydroxides; or such organic bases as isopropylamine, trimethylamine, histidine, or procaine.

In some aspects, a pharmaceutical lipid vehicle composition that includes polypeptides, one or more lipids, and an aqueous solvent may be used. As used herein, the term “lipid” refers to any of a broad range of substances that are characteristically insoluble in water and extractable with an organic solvent. This broad class of compounds is well known to those of skill in the art, and as the term “lipid” is used herein, it is not limited to any particular structure. Examples include compounds that contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether- and ester-linked fatty acids, polymerizable lipids, and combinations thereof. Of course, compounds other than those specifically described herein that are understood by one of skill in the art as lipids are also encompassed by the compositions and methods. One of ordinary skill in the art would be familiar with the range of techniques that can be employed for dispersing a composition in a lipid vehicle. For example, the antibody may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The term “unit dose” or “dosage” refers to physically discrete units suitable for use in a subject, each unit containing a predetermined quantity of the therapeutic antibody or composition containing the therapeutic antibody calculated to produce the desired responses discussed above in association with its administration, i.e., the appropriate route and treatment regimen. The quantity to be administered, both according to number of treatments and unit dose, depends on the effect desired. The actual dosage amount of a composition as described herein that can be administered to a patient or subject can be determined by physical and physiological factors, such as body weight, the age, health, and sex of the subject, the type of disease being treated, the extent of disease penetration, previous or concurrent therapeutic interventions, idiopathy of the subject, the route of administration, and the potency, stability, and toxicity of the particular therapeutic substance. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 milligram/kg/body weight or more per administration, and any range derivable therein. In non-limiting examples of a derivable range from the numbers listed herein, a range of about 5 milligram/kg/body weight to about 100 milligram/kg/body weight, about 5 microgram/kg/body weight to about 500 milligram/kg/body weight, etc., can be administered, based on the numbers described above. The foregoing doses include amounts between those indicated and are intended to also include the lower and upper values of the ranges. The practitioner responsible for administration will, in any event, determine the concentration of active ingredient(s) in a composition and appropriate dose(s) for the individual subject.

The particular nature of the therapeutic composition or preparation is not intended to be limiting. For example, suitable compositions may be provided in formulations together with physiologically tolerable liquid, gel, or solid carriers, diluents, and excipients. In some aspects, the therapeutic preparations may be administered to mammals for veterinary use, such as with domestic animals, and clinical use in humans in a manner similar to other therapeutic agents. In general, the dosage required for therapeutic efficacy will vary according to the type of use and mode of administration, as well as the particularized requirements of individual subjects, as described supra.

CCL21 as a biomarker. Disclosed herein are methods comprising the use of at least one anti-CCL21 antibody as described herein. In some aspects, the methods can comprise detecting the amount of level of CCL21 gene or protein in a sample obtained from a subject who has a cancer or tumor or is exhibiting one or more symptoms of an autoimmune disease or disorder or is an allograft transplant recipient. Such methods may be useful in biomarker evaluations of the level of CCL21 in a sample obtained from a subject who has a cancer or tumor or is exhibiting one or more symptoms of an autoimmune disease or disorder or is an allograft transplant recipient. In some aspects, the autoimmune disease or disorder is inflammatory bowel disease. For example, if the subject's sample is tested and determined to comprise a higher level of CCL21 compared to a reference sample, then the subject is a candidate for treatment with an anti-CCL21 antibody as described herein, alone, or in combination with another agent, for example, would benefit from the treatment. Such methods comprise obtaining a sample from a subject having a cancer or tumor (or exhibiting one or more symptoms of an autoimmune disease or disorder or is an allograft transplant recipient), testing the sample for the presence of CCL21 derived from the subject's sample using binding methods known and used in the art and as described herein, for example, using an anti-CCL21 antibody as described herein, and administering to the subject an effective amount of an anti-CCL21 antibody alone, or in combination with another agent, if the subject's sample is found to have a higher level of CCL21 when compared to a reference sample. Diagnosing the subject as having a cancer or tumor or an autoimmune disease prior to treatment allows for more effective treatment and benefit to the subject, as the administered anti-CCL21 antibody is more likely to block or inhibit the interaction of the subject's CCL21 with the subject's CCR7, thereby inducing immunosuppression of the T-cell activity or reducing T-cell activation or blocking T-cell migration or adherence. In some aspects, the methods can involve first selecting a subject whose cancer or tumor or disease state or condition may be amenable to testing for the presence of CCL21 levels.

Similar methods may be used to monitor the presence of CCL21 levels during a course of treatment or therapy, including combination treatments with an anti-CCL21 antibody and another anticancer drug or treatment or another immunosuppressant, over time, as well as after treatment has ceased. Such methods may also be used in companion diagnostic methods in which a treatment regimen or combination treatment, involves testing or assaying a sample obtained from the subject for CCL21 levels, prior to treatment and during the course of treatment, e.g., monitoring, to determine a successful outcome or the likelihood thereof.

Other Agents. It is contemplated that other agents may be used in combination with certain aspects of the compositions and methods disclosed herein to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions may increase the anti-hyperproliferative effects on the neighboring hyperproliferative cell population. In some aspects, cytostatic or differentiation agents may be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

Fusions and Conjugates

The anti-CCL21 antibodies or polypeptides disclosed herein can also be expressed as fusion proteins with other proteins or chemically conjugated to another moiety. In some aspects, the antibodies or polypeptides can have an Fc portion that can be varied by isotype or subclass, can be a chimeric or hybrid, and/or can be modified, for example to improve effector functions, control half-life or tissue accessibility, augment biophysical characteristics, such as stability, and improve efficiency of production, which can be associated with cost reductions. Many modifications useful in the construction of fusion proteins and methods for making them are known in the art, for example, as reported by Mueller, J. P. et al., 1997, Mol. Immun. 34(6):441-452; Swann, P. G., 2008, Curr. Opin. Immunol., 20:493-499; and Presta, L. G., 2008, Curr. Opin. Immunol., 20:460-470. In some aspects, the Fc region can be the native IgG1, IgG2, or IgG4 Fc region of the antibody. In some aspects, the Fc region can be a hybrid, for example, a chimera containing IgG2/IgG4 Fc constant regions. Modifications to the Fc region include, but are not limited to, IgG4 modified to prevent binding to Fc gamma receptors and complement; IgG1 modified to improve binding to one or more Fc gamma receptors; IgG1 modified to minimize effector function (amino acid changes); and IgG1 with altered pH-dependent binding to FcRn. The Fc region can include the entire hinge region, or less than the entire hinge region of the antibody.

In some aspects, IgG2-4 hybrids and IgG4 mutants have reduced binding to FcR which can increase their half-life. Representative IG2-4 hybrids and IgG4 mutants are described, for example, in Angal et al., 1993, Molec. Immunol., 30(1):105-108; Mueller et al., 1997, Mol. Immun., 34(6):441-452; and U.S. Pat. No. 6,982,323; all of which are hereby incorporated by references in their entireties. In some aspects, the IgG1 and/or IgG2 domain can be deleted. For example, Angal et al., Id., describe proteins in which IgG1 and IgG2 domains have serine 241 replaced with a proline. In some aspects, fusion proteins or polypeptides having at least 10, at least 20, at least 30, at least 40, at least 50, at least 60, at least 70, at least 80, at least 90 or at least 100 amino acids are contemplated.

In some aspects, anti-CCL21 antibodies or polypeptides can be linked to or covalently bind or form a complex with at least one moiety. Such a moiety may be, but is not limited to, one that increases the efficacy of the antibody as a diagnostic or a therapeutic agent. In some aspects, the moiety can be an imaging agent, a toxin, a therapeutic enzyme, an antibiotic, a radio-labeled nucleotide, a chemotherapeutic agent, and the like.

In some aspects, antibodies and polypeptides as described herein may be conjugated to a marker, such as a peptide, to facilitate purification. In some aspects, the marker can be a hexa-histidine peptide, i.e., the hemagglutinin “HA” tag, which corresponds to an epitope derived from the influenza hemagglutinin protein (Wilson, I. A. et al., Cell, 37:767-778 (1984)), or the “flag” tag (Knappik, A. et al., Biotechniques 17(4):754-761 (1994)).

In some aspects, the moiety conjugated to the antibodies and polypeptides as described herein can be an imaging agent that can be detected in an assay. Such imaging agents can be enzymes, prosthetic groups, radiolabels, nonradioactive paramagnetic metal ions, haptens, fluorescent labels, phosphorescent molecules, chemiluminescent molecules, chromophores, luminescent molecules, bioluminescent molecules, photoaffinity molecules, or colored particles or ligands, such as biotin. In some aspects, suitable enzymes include, but are not limited to, horseradish peroxidase, alkaline phosphatase, beta-galactosidase, or acetylcholinesterase; prosthetic group complexes include, but are not limited to, streptavidin/biotin and avidin/biotin; fluorescent materials include, but are not limited to, umbelliferone, fluorescein, fluorescein isothiocyanate, rhodamine, dichlorotriazinylamine fluorescein, dansylchloride or phycoerythrin; luminescent materials include, but are not limited to, luminol; bioluminescent materials include, but are not limited to, luciferase, luciferin, and aequorin; radioactive materials include, but are not limited to, bismuth (²¹³Bi), carbon (¹⁴C), chromium (⁵¹Cr), cobalt (⁵⁷Co), fluorine (¹⁸F), gadolinium (¹⁵³Gd, ¹⁵⁹Gd), gallium (⁶⁸Ga, ⁶⁷Ga), germanium (⁶⁸Ge), holmium (¹⁶⁶Ho) indium (¹¹⁵In, ¹¹³In, ¹¹²In, ¹¹¹In), iodine (¹³¹I, ¹²⁵I, ¹²³I, ¹²¹I), lanthanium (¹⁴⁰La), lutetium (¹⁷⁷Lu), manganese (⁵⁴Mn), molybdenum (⁹⁹Mo), palladium (¹⁰³Pd), phosphorous (³²P), praseodymium (¹⁴²Pr), promethium (¹⁴⁹Pm), rhenium (¹⁸⁶Re, ¹⁸⁸Re), rhodium (¹⁰⁵Rh), ruthenium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹Yb, ¹⁷⁵Yb), yttrium (⁹⁰Y), zinc (⁶⁵Zn); positron emitting metals using various positron emission tomographies, and nonradioactive paramagnetic metal ions.

The imaging agent can be conjugated to the antibodies or polypeptides described herein either directly or indirectly through an intermediate (such as, for example, a linker known in the art) using techniques known in the art. See, for example, U.S. Pat. No. 4,741,900 which reports on metal ions that can be conjugated to antibodies and other molecules as described herein for use as diagnostics. Some conjugation methods involve the use of a metal chelate complex employing, for example, an organic chelating agent, such as diethylenetriaminepentaacetic acid anhydride (DTPA); ethylenetriaminetetraacetic acid; N-chloro-p-toluenesulfonamide; and/or tetrachloro-3-6α-diphenylglycouril-3, attached to the antibody. Monoclonal antibodies can also be reacted with an enzyme in the presence of a coupling agent such as glutaraldehyde or periodate. Conjugates with fluorescein markers can be prepared in the presence of these coupling agents or by reaction with an isothiocyanate.

In some aspects, the anti-CCL21 antibodies polypeptides as described herein can be conjugated to a second antibody to form an antibody heteroconjugate, for example, as described in U.S. Pat. No. 4,676,980. Such heteroconjugate antibodies can additionally bind to haptens (e.g., fluorescein), or to cellular markers.

In some aspects, the anti-CCL21 antibodies or polypeptides described herein can also be attached to solid supports, which can be useful for carrying out immunoassays or purification of the target antigen or of other molecules that are capable of binding to the target antigen that has been immobilized to the support via binding to an antibody or antigen binding fragment as described herein. Such solid supports include, but are not limited to, glass, cellulose, polyacrylamide, nylon, polystyrene, polyvinyl chloride or polypropylene.

Kits and Diagnostics

Disclosed herein are kits comprising therapeutic agents and/or other therapeutic and delivery agents. In some aspects, the kits can be used for preparing and/or administering a therapy involving the anti-CCL21 antibodies described herein. The kits can comprise one or more sealed vials containing any of the pharmaceutical compositions as described herein. The kits can include, for example, at least one CCL2 antibody, as well as reagents to prepare, formulate, and/or administer one or more anti-CCL21 antibodies or to perform one or more steps of the described methods. In some aspects, the kits can also comprise a suitable container means, which is a container that will not react with components of the kit, such as an Eppendorf tube, an assay plate, a syringe, a bottle, or a tube. The container may be made from sterilizable materials, such as plastic or glass.

The kits can further include an instruction sheet that outlines the procedural steps of the methods set forth herein, and will follow substantially the same procedures as described herein or are known to those of ordinary skill. The instruction information may be in a computer readable medium containing machine-readable instructions that, when executed using a computer, cause the display of a real or virtual procedure of delivering a pharmaceutically effective amount of the therapeutic agent.

### EXAMPLES

### Example 1: Targeting Chemokine CCL21 with a Monoclonal Antibody in Inflammatory Bowel Diseases

Also disclosed herein is a mouse monoclonal antibody (mAb) that neutralizes human CCL21. CCL21 is a chemokine that is used to attract naïve T cells, B cells and dendritic cells to sites of inflammation. These cells are recruited by CCL21 interaction with the receptor CCR7 on the surface of responding cells, guiding these cells to the site of inflammation. By neutralizing CCL21, tissue infiltration and destruction mediated by T cells, such as that seen in autoimmune disorders, can be limited.

To mediate disease progression T-cells need to recognize the specific auto-antigen, however T cells are unable to recognize these peptides on their own. Rather, they need the autoantigen presented to them by professional antigen-presenting cells (APCs) like dendritic cells (DCs). This autoantigen presentation typically occurs at the target-tissue site or its respective draining lymph node. Once these autoreactive T cells are primed, they proliferate and develop inflammatory or cytotoxic phenotypes which can mediate tissue destruction and recruit other immune cells to the target tissue. This creates a cyclical process as the disease progresses, releasing more autoantigen, priming or re-priming T cells and perpetuating inflammation as well as tissue destruction. The infiltration and recruitment of immune cells into the target-tissue microenvironment is an important step of many autoimmune diseases that is directed by a network of chemotactic cytokines (chemokines) that guide specific cells.

Chemokines are a family of small proteins that share structural and functional elements. They share cysteine-mediated covalent bonds in the amino terminus and mediation of leukocyte migration (Hughes C E, Nibbs R J B. FEBS J. 2018 August; 285(16):2944-2971; and Hernandez-Ruiz M, Zlotnik A. J Interferon Cytokine Res. 2017 February; 37(2):62-70). Chemokines and their receptors are important in many types of human diseases, playing important roles in inflammatory tissue destruction seen in atherosclerosis, adult respiratory distress syndrome, cerebral vascular events, and myocardial infarction (Bryant V L, Slade C A. Immunol Cell Biol. 2015 April; 93(4):364-7; Kolaczkowska E, Kubes P. Nat Rev Immunol. 2013 March; 13(3):159-75; and Sakai Y, Kobayashi M. Pathol Int. 2015 July; 65 (7):344-54). Chemokines have been shown to mediate the inhibition of hematopoiesis during systemic inflammation (Broxmeyer H E, et al. Ann N Y Acad Sci. 1999 Apr. 30; 872:142-62). In addition, chemokines can also promote the aberrant migration of leukocytes into target organs during autoimmune diseases, such as lupus, rheumatoid arthritis, and inflammatory bowel disease (Sakai Y, Kobayashi M. Pathol Int. 2015 July; 65 (7):344-54; Wang L, et al. J Intern Med. 2015 October; 278(4):369-95; and Singh U P, et al. Cytokine. 2016 January; 77: 44-9).

A CC chemokine designated CCL21 has been cloned and characterized and shown to be a potent chemo-attractant for naïve T-cells and immature dendritic cells (Hromas R, et al. J Immunol. 1997; 159:2554-2558; Hedrick J A, Zlotnik A. J Immunol. 1997; 159:1589-1593; and Nagira M, et al. J Biol Chem. 1997; 272:19518-19524). The carboxy terminus comprises 37 amino acids that is acidic in nature, and interacts with heparinoids on endothelial cell surfaces to immobilize it. The amino terminus then can interact with CCR7, the cognate receptor for CCL21. This chemokine is specific for attracting naïve B- and T-cells and immature dendritic cells, for antigen presentation by dendritic cells. CCL21 directed migration of naive B-cells and natural killer (NK) cells, although to a lesser extent than T-cells (Hromas R, et al. J Immunol. 1997; 159:2554-2558; Hedrick J A, Zlotnik A. J Immunol. 1997; 159:1589-1593; and Nagira M, et al. J Biol Chem. 1997; 272:19518-19524). It does not direct migration of monocytes or granulocytes (Hromas R, et al. J Immunol. 1997; 159:2554-2558; Hedrick J A, Zlotnik A. J Immunol. 1997; 159:1589-1593; and Nagira M, et al. J Biol Chem. 1997; 272:19518-19524). CCL21 also promotes naïve lymphocyte adhesion to the endothelium of small venules, most notably in lymph nodes where naïve lymphocytes can be presented with antigen, but also in primary inflamed tissues as well (Comerford I, et al. Cytokine Growth Factor Rev. 2013 June; 24(3):269-83; Förster R, et al. Nat Rev Immunol. 2008 May; 8(5):362-71; and Gunn M D, et al. Proc Natl Acad Sci USA. 1998; 95:258-263). The expression of CCL21 in the high endothelial venules of lymph nodes therefore mediates naïve T-cell trafficking to secondary lymphoid organs for antigen presentation.

CCL21 has been shown to be over-expressed in the endothelial cells of tissue inflamed by auto-immune infiltration of T-cells. Thus, preventing its interaction with its cellular receptor, CCR7, might decrease the lymphocytic infiltration that causes tissue damage in auto-immune diseases. Targeting it with a humanized monoclonal antibody may prevent auto-immune disease damage. In skin autoimmune disease, CCL21 has been shown to be was highly induced in the venule endothelium of the inflamed tissue but not in normal tissue (Christopherson K W 2nd, et al. Blood. 2003 Feb. 1; 101(3):801-6). For example, it has been shown that CCL21 is not expressed in samples of healthy skin but is expressed in venule endothelial cells of atopic dermatitis, lichen planus, and graft-versus-host disease by immunohistology.

In addition, the vast majority of the infiltrating T-cells expressed CCR7, the receptor for CCL21. This raised the possibility that local auto-antigen presentation in the primary inflamed tissue could be more important than secondary lymphoid tissue auto-antigen presentation in generating local tissue damaging effector T-cells (Comerford I, et al. Cytokine Growth Factor Rev. 2013 June; 24(3):269-83; Förster R, et al. Nat Rev Immunol. 2008 May; 8(5):362-71; Gunn M D, et al. Proc Natl Acad Sci USA. 1998; 95:258-263; and Christopherson K W 2nd, et al. Blood. 2003 Feb. 1; 101(3):801-6).

Using a murine skin hypersensitivity model, it was found that endothelial CCL21 was induced rapidly in skin venule endothelial cells after re-challenge with the topical antigen. For these experiments, unchallenged normal ear skin small vessels and endothelium, and immunohistologic analysis of CCL21 expression in the challenged hyper-sensitized skin were used. The results demonstrated that small venule endothelium has induced expression of CCL21 upon antigen stimulation of inflammation. These studies imply that expression of venule CCL21 is an important chemoattractant for helper T-cells to enter inflamed tissue in autoimmune diseases. Thus, interrupting this T-cell chemoattraction could improve the biology of these auto-immune diseases, limiting disease progression and restricting tissue damage.

A large need exists for directed therapies with specific mechanisms of action in the field of autoimmunity and immune over activation. Standard treatment options include broadly immunosuppressive drugs that leave patients susceptible to numerous infections and viruses. mAbs against specific immune targets have begun to address these needs. T cell mediated autoimmunity and inflammation characterize a large class of diseases and disorders that require targeted pharmaceuticals in order to increase patient responsiveness, maximize their quality of life and minimize the risk of cancer (Gajendran, Mahesh, et al. Disease-a-Month, vol. 65, no. 12, 2019, p. 100851). Inflammatory bowel diseases (IBDs) broadly describe a cluster of T cell mediated disorders, primarily including Crohn's disease (CD) and ulcerative colitis (UC). As of 2019, IBD was still one of the most pressing T-cell mediated autoimmune diseases, collectively impacting ˜4.65 million patients in the U.S. and 13.76 million patients in the seven major markets (7MM; US, 5EU, Japan). These disorders have a higher incidence rate in industrialized countries such as the U.S. and EU, as primary environmental risk factors include the adoption of a western diet, smoking and ethnicity (Feuerstein, Joseph D., and Adam S. Cheifetz. Mayo Clinic Proceedings, Vol. 92, No. 7, 2017, pp. 1088-1103).

While progress has been made, changing the history of inflammatory bowel diseases (IBD) is still problematic because the initiators of the disease are imperfectly defined (Yao D, et al. Inflamm Bowel Dis. 2019 Jul. 9. pii: izz149; and Zhang Y Z, Li Y Y. World J Gastroenterol. 2014 Jan. 7; 20(1):91-9). The goal of interrupting the signaling cascade that leads to the lymphocytic destruction of the intestinal mucosa in these diseases has been hampered not just by the lack of targets but by the ineffectiveness of therapies against the targets that are known (Sakai Y, Kobayashi M. Pathol Int. 2015 July; 65 (7):344-54; Singh U P, et al. Cytokine. 2016 January; 77: 44-9; Yao D, et al. Inflamm Bowel Dis. 2019 Jul. 9. pii: izz149; and Zhang Y Z, Li Y Y. World J Gastroenterol. 2014 Jan. 7; 20(1):91-9). Since the gut is its own secondary lymphoid organ where naïve lymphocytes can be presented antigen locally (instead of maturing in a secondary lymphoid organ and then migrating to target inflamed/infected tissues), one potential IBD therapeutic target could be interrupting the migration of naïve lymphoid cells into the inflamed gut (Sakai Y, Kobayashi M. Pathol Int. 2015 July; 65 (7):344-54; Wang L, et al. J Intern Med. 2015 October; 278(4):369-95; and Singh U P, et al. Cytokine. 2016 January; 77: 44-9; and Yao D, et al. Inflamm Bowel Dis. 2019 Jul. 9. pii: izz149). CCL21 expression correlated with induction of ulcerative colitis in mice, and when that colitis was treated CCL21 expression decreased (Singh U P, et al. Cytokine. 2016 January; 77: 44-9; and Zhang H, et al. Genet Mol Res. 2014 Apr. 29; 13(2):3337-45). Therefore, it was tested whether blocking CCL21-directed migration of naïve immune cells might alter the course of IBD (Danese S, Fiocchi C. Dig Dis. 2016; 34(1-2):43-50).

As described herein, a series of monoclonal antibodies to the amino terminus of human CCL21 were generated and one or more clones that completely block naïve T-cell migration towards CCL21 were identified. However, migration of memory T-cell towards CCL21 was less affected. Using this monoclonal CCL21 antibody, the results demonstrated that CCL21 is expressed in the mucosal venule endothelium of the majority of inflammatory bowel diseases, including Crohn's disease, ulcerative colitis, and celiac disease. Thus, in some aspects, the CCL21 antibody described herein could be used to prevent initiation or recurrence of inflammatory bowel diseases by selectively interfering with recruitment of naïve immune effector cells to sites of antigen presentation, without harming overall memory immunity (Yao D, et al. Inflamm Bowel Dis. 2019 Jul. 9. pii: izz149; Zhang Y Z, Li Y Y. World J Gastroenterol. 2014 Jan. 7; 20(1):91-9; Zhang H, et al. Genet Mol Res. 2014 Apr. 29; 13(2):3337-45; and Danese S, Fiocchi C. Endothelial Cell-Immune Cell Interaction in IBD. Dig Dis. 2016; 34(1-2):43-50).

The current standard of care for IBD includes broadly immunosuppressive treatments as 5-ASAs, steroids, thiopurines, or directed biologics (anti-TNF anti-interleukin or anti-integrin mAbs). However, there are no currently FDA approved biologics available for the treatment of Crohn's disease. In 2019, the global Crohn's disease and ulcerative colitis biologics market in the 7MM were valued at $5.89 billion and $3.55 billion, respectively, and are estimated to reach $10.06 billion by 2026, which is largely driven by increase in disease prevalence, high treatment rates, the uptake of oral therapies and the approval of 9 pipeline therapies.

Currently, there are no anti-CCL21 agents approved by the FDA nor are there any ongoing clinical trials using anti-human CCL21 mAbs. The anti-human CCL21 mAb disclosed herein can be used in the treatment of T-cell autoimmune diseases including but not limited to IBD, Crohn's disease, T1D, RA, Psoriasis, and MS. The monoclonal antibodies described herein can decrease the tissue destruction seen in multiple autoimmune diseases where T-cells play the major role in mediating tissue destruction. Examples include but are not limited to Crohn's disease, ulcerative colitis, the two types of inflammatory bowel disease, where T-cell promote the severity of the erosion of the gut mucosa, Graft Versus Host Disease, where T-cells destroy the gut mucosa, the hepatic biliary tree, the skin, scleroderma, where T-cells may mediate the epidermal destruction and subsequent fibrosis, and psoriasis, where T-cell infiltration of the skin produces the tissue damage seen in this disease. In addition, some types of rejection of solid organ transplants such as kidney or liver transplantation that may be mediated by T-cell cytotoxicity, may also respond to CCL21 monoclonal antibodies. Blocking T-cell infiltration into the sites of organ damage may slow or stop such damage in these diseases. In the past, treatment for these diseases has resulted in general immunosuppression of the patient, with many morbid side-effects, especially life-threatening infections. Steroids commonly used in these diseases can also produce muscle wasting, osteoporosis, and decreased general immunity leading to infections. An agent that could specifically inhibit naive T-cell movement into the site of inflammation and their maturation into tissue destroying T-cells, but not inhibit mature T-cell function or movement in non-inflamed regions, can be effective in treating these diseases. The selectivity of CCL21 for naïve T-cells means that inhibiting it would not harm the mature immune system, reducing the risk of infections for this therapy. Anti-CCL21 may also be important for sustaining remissions.

Materials and Methods

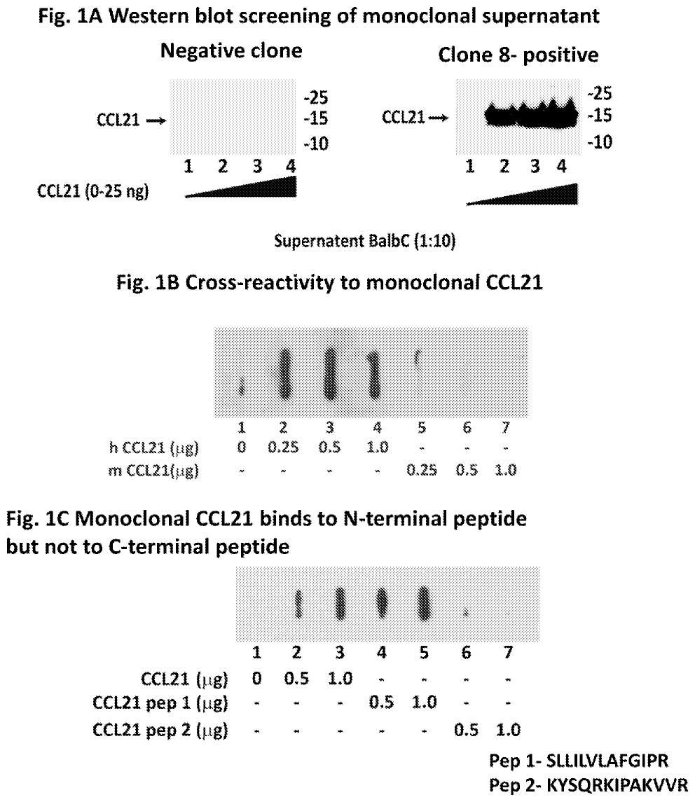

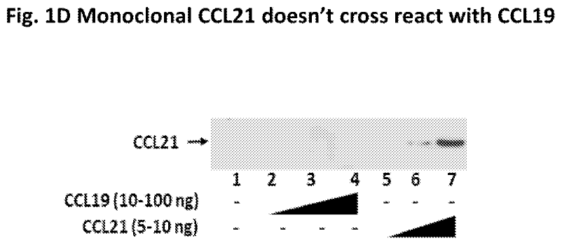

Monoclonal antibody generation. BALB/C mice were subcutaneously immunized with 2 mg each of human CCL21 recombinant full-length protein in complete Freund's adjuvant and boosted three times with the equivalent preparation before splenic harvest. 196 fused plasma cell clones screened by ELISA that produced an antibody that reacted with human CCL21 for binding to the amino terminus of human CCL21 protein. The monoclonal antibody clones were screened for whether a peptide from the CCR7-interacting region of CCL21 could compete off the monoclonal from binding CCL21. More specifically, it was tested whether two peptides from the amino terminus of hCCL21, which interacts with CCR7, could compete off the antibody clone binding to CCL21 using slot blots (FIG. 1).

Human Peripheral Blood T Cell Isolation. Normal donor human peripheral blood (PB) was collected. Mononuclear cell layer was collected using Ficoll-Paque PLUS (GE Healthcare Bio-Sciences AB; Pittsburgh, PA) density gradient centrifugation. The CD3+ PB cells were then isolated using immunoaffinity selection with MiniMACS paramagnetic CD3 microbeads (Miltenyi Biotec; Auburn, CA) using two sequential LS columns (Miltenyi Biotec; Auborn, CA).

T-Cell Chemotaxis Assay. T-cell chemotaxis was measured (Hromas R, et al. J Immunol. 1997; 159:2554-2558). Human PB T cells acclimated to 37° C. were suspended in prewarmed RPMI (37° C.) with 0.5% bovine serum albumin (BSA; Sigma-Aldrich; St. Louis, MO). Costar 24-well transwell plates with 6.5 mm diameter inserts with 5.0 μm pores (Sigma-Aldrich; St. Louis, MO) were prepared by placing 650 μl of prewarmed RPMI with BSA that contained 0, 1200 ng/mL rhCCL21 (R&D Systems, Minneapolis, MN) or 1200 ng/mL rhCCL21 pretreated for 1 hour with sample clones #7-39 in the bottom well and allowing plates to acclimate at 37° C. for half an hour prior to chemotaxis assay. Cells were suspended at 300,000 cells/100 μl prewarmed RPMI with 0.5% BSA and loaded to the top chamber of the transwell assay. Transwell plates were placed in a 37° C. incubator (95% humidity, 5% CO₂) for 4 hours. Percent migration was determined using flow cytometry (counts determined by running samples for the same amount of time at the same speed) with background migration (cells that migrated toward media alone; always <4%) subtracted from total migrated cells.

Flow Cytometry. To analyze the T cell populations which migrated in the chemotaxis assays, after counting by flow (see herein), cells were washed in PBS, incubated in fluorescently conjugated anti-human antibody cocktail for 20 minutes at room temperature, washed in PBS, and then fixed in 1% formaldehyde. The samples were analyzed on an LSR II flow cytometer (BD Biosciences). Antibody concentrations were used as stated by manufacturer's instructions. Data analysis was performed using FlowJo 7.6.3 software (Tree Star). Gates were determined using fluorescence minus one control. The following markers were used: APC-H7 conjugated anti-human CD3 (clone SK7), PerCP-Cy5.5 conjugated anti-human CD4 (clone SK3), FITC conjugated anti-human CD45RA (clone HI100), Alexa Fluor® 647 conjugated anti-human CD197 (CCR7; clone 150503) [components of a Human Naïve/Memory T cell Panel Kit from BD Biosciences cat. #561438; San Jose, CA], BV421 conjugated anti-human CD8 (clone RPA-T8; BD Biosciences; San Jose, CA), PE conjugated anti-human CD27 (clone M-T271; BD Biosciences; San Jose, CA). For the antibodies used in these studies, the validation for the relevant species and applications can be found on the indicated manufacturer's website.

T-cell chemotaxis. T-cell chemotaxis was measured (Hromas R, et al. J Immunol. 1997; 159:2554-2558). Briefly, human peripheral blood (PB) was separated for mononuclear cells via Ficoll gradient separation then CD3+ cells were isolated via positive selection using a MACs magnetic bead kit. 24-well Transwell® plates with 6.5 mm diameter inserts and 5.0 μM pore size were prepared by placing 650 μL of pre-warmed (37° C.) serum-free RMPI with BSA that contained no chemokine, 1200 ng/mL rhCCL21 or 1200 ng/mL rhCCL21 pretreated for 1 hour with clone #8 in the bottom well and allowing plates to acclimate at 37° C. for half an hour prior to chemotaxis assay. PB CD3+ cells were resuspended at 300,000 cells/100 μL prewarmed RPMI with 0.5% BSA and loaded to the top chamber of the Transwell® assay. Plates were placed in a 37° C. incubator for 4 hours. Percent migration of CD3+, CD3+CD4+, CD3+CD4+CD8−, and then CCR7/CD45RA/CD27 subsets of the above were determined using flow cytometry (20). Background migration (cells that migrated toward media with no chemokine) was subtracted from total cell migration. Data are the mean±SD of triplicate wells. Data is the representative of two experiments.

Immunohistology. Immunohistology was performed (Christopherson K W 2nd, et al. Blood. 2003 Feb. 1; 101(3):801-6). The anatomic pathology database was searched for cases of psoriasis (positive controls), inflammatory bowel disease (Crohn's disease and ulcerative colitis), celiac sprue, and rheumatoid arthritis (negative controls). Hematoxylin and eosin-stained slides were reviewed to confirm diagnoses in order to select blocks for immunohistochemical staining. For the cases, 4-micron thick sections were cut from the paraffin-embedded formalin-fixed tissue blocks and placed on charged slides. Sections were deparaffinized in xylene and rehydrated through graded alcohols to distilled water before undergoing antigen retrieval. Immunohistochemistry was performed using the Discovery Ultra automated instrument (Roche, Indianapolis, IN) per the manufacturer's instructions, with CCL21-1E7 antibody clone at a 1:1000 dilution, and anti-mouse conjugated with DAB. Positive controls of normal human lymph node and negative controls of like tissue with mouse IgG were also performed. Immunoreactivity was qualitatively assessed by 2 pathologists. Counter-staining was performed with hematoxylin and eosin. Reactions were judged negative if there was no capillary endothelial expression, just rare capillaries expressing, or weak or blush discontinuous expression. Staining was read blind to diagnoses, and classified as positive if there was multifocal, strong, and nearly circumferential capillary endothelial expression.

Results

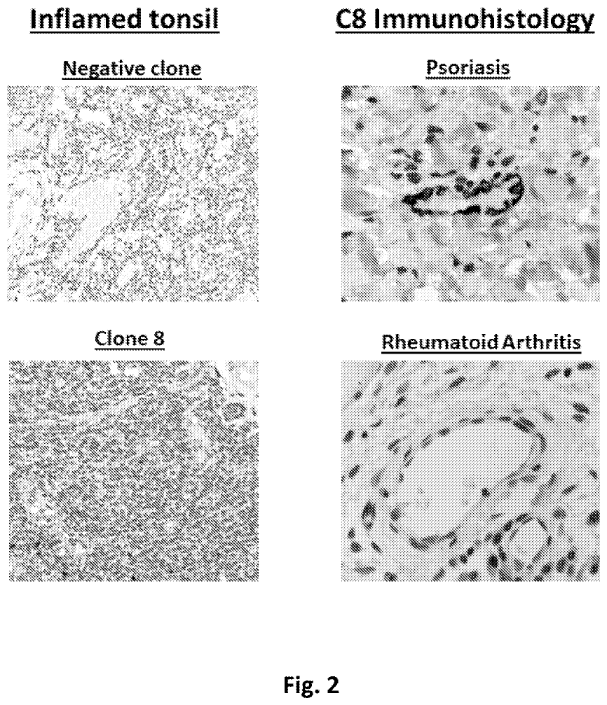

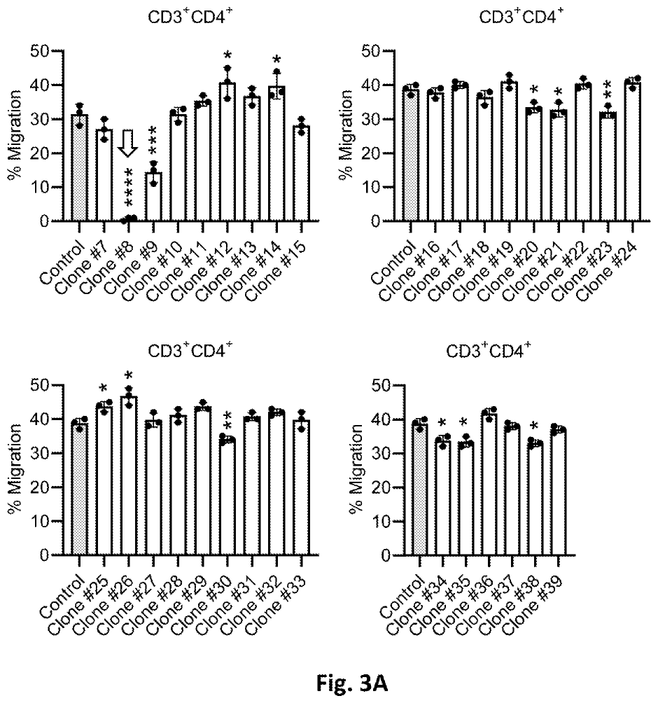

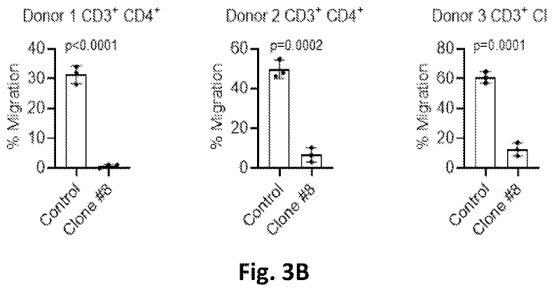

Generation of monoclonal antibodies against the amino terminus of human CCL21. BALB/C mice were subcutaneously immunized with human CCL21 recombinant full-length protein in adjuvant and boosted three times before splenic harvest. 196 fused plasma cell clones were screened by ELISA that produced an antibody that reacted with human CCL21 for western blot recognition of human CCL21 protein as a single band (FIG. 1). Using slot blot analysis, titer and specificity of these antibodies was screened (FIG. 1). High titer antibodies that bound specifically to human CCL21 and not mouse CCL21 were assessed. Next, clones were screened for whether they interacted with the amino terminal CCR7-binding region of CCL21 using slot blot analysis. The amino terminus of CCL21 is the protein region that interacts with its receptor CCR7 (21). Further screening was performed by assessing whether peptides homologous to the amino terminus of CCL21 could compete off the monoclonal antibody clones in the slot blot analysis. Peptides containing amino acids 8-20 (Pep 1) and 43-56 (Pep 2) of CCL21 were used to assess binding of the monoclonal antibody clones to regions of CCL21 that interact with the CCR7 receptor (an example slot blot is provided in FIG. 1) (21). Clones were then subjected to immunohistologic analysis for binding to the appropriate regions of an inflamed lymph node (FIG. 2). After these screens were complete, 33 clones were left for analysis that selectively bound to the regions of CCL21 that interacted with CCR7 at a high titer. These clones were then assessed for inhibition of T-cell migration in transwell chemotaxis assays shown in FIG. 3. Clone 8 as well as 32 other clones were positive in binding to CCL21 in human tonsil lymph node and also fulfilled the screening criteria in FIG. 1.

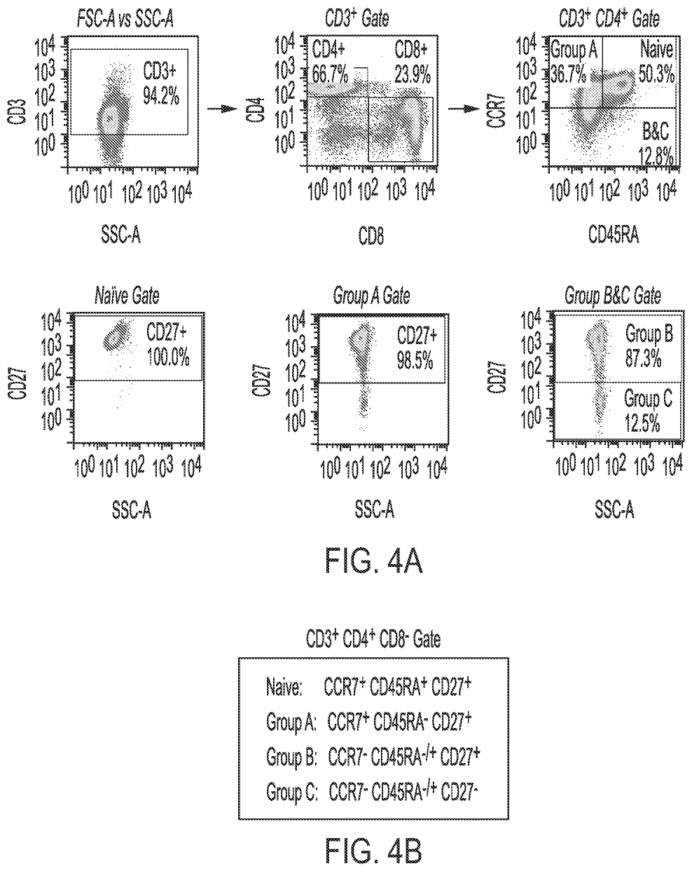

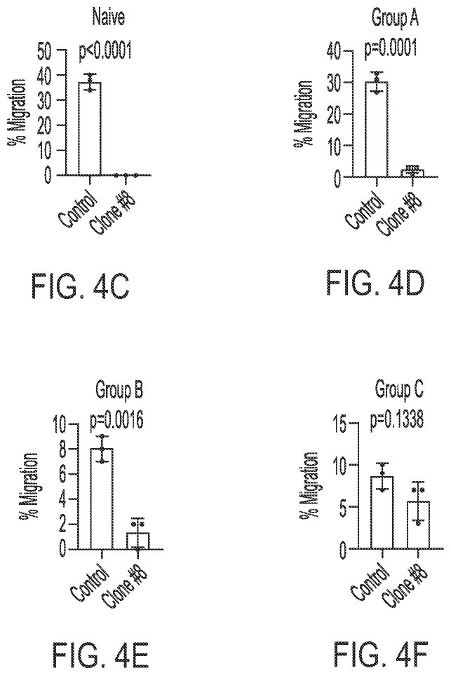

Monoclonal antibody blockade of human CCL21 function. Using transwell chemotaxis assays, the ability of each of the 33 monoclonal antibody clones to block CD3+CD4+ helper (h) T-cell migration from the top well to the lower well in response to a CCL21 gradient was measured (FIG. 3). A clone (Clone #8, termed C8 here) was identified that completely abrogated Th-cell chemotaxis towards CCL21 and another clone (Clone #9) was identified that partially blocked Th-cell chemotaxis towards CCL21. None of the other clones tested showed any significant inhibitory activity. Clones 8 and 9 were found to have the same variable region sequence, indicating that they had been derived from the same B-cell clone in the immunized mouse. Interestingly, clones #12-14, 25, 26 each enhanced chemotaxis towards CCL21 (FIG. 4A,C), implying that they improved presentation of CCL21 to its receptor CCR7 on Th-cells.

CCL21 promotes migration of naive helper T-cells but not memory T-cells-Flow cytometric analysis of T-cell subsets (example of gating strategy provided in FIG. 4) was used to assess the type of T-cell that responds to CCL21 and was blocked by anti-CCL21 C8 (FIG. 4). CD45RA and CD27 expression define naïve T-cells which have not yet been exposed to or responded to antigen (20; FIG. 4G). The results show that CD3+CD4+CD45RA+CD27+ naïve Th-cells migrated best to CCL21 and that this migration was nearly completely blocked by C8 (FIG. 4C,D). However, Th-cells that do not express CD27, which are cells that may have already responded to antigen, migrate with significantly less frequency to CCL21, and C8 was less successful in inhibiting migration (FIGS. 4E, F).

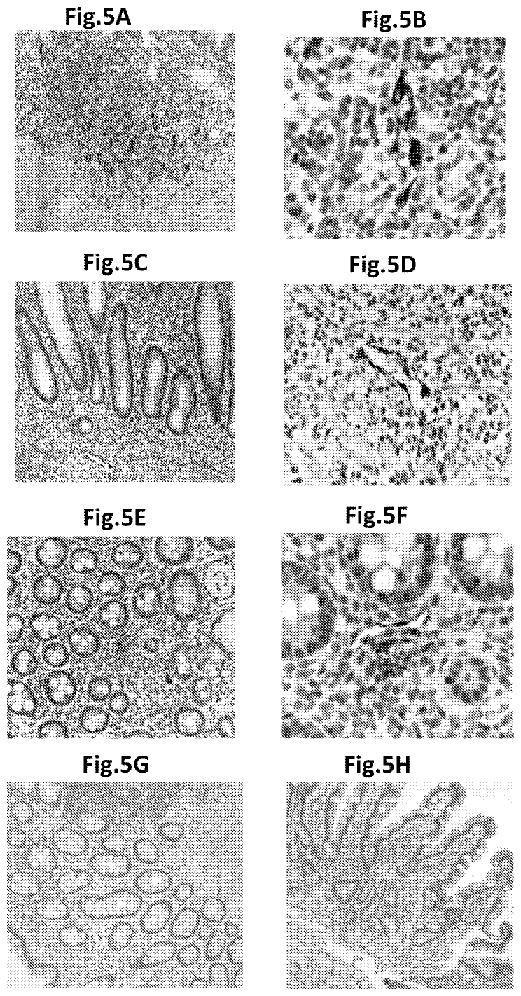

Expression of CCL21 in the endothelium of target tissues in intestinal autoimmune diseases. The binding of C8 was examined in biopsy specimens of intestinal autoimmune diseases (Crohn's disease, ulcerative colitis, and celiac sprue disease) using immunohistology (FIG. 5). Psoriasis was used as a positive control, since it is known that T-cell-infiltrative autoimmune diseases of the skin expressed CCL21 in the venule endothelium (15). Rheumatoid arthritis was used as a negative control, since this T-cell infiltration in this autoimmune disease is mediated by a distinct mechanism (7). CCL21 expression in the dermal venule endothelium was recognized by C8 in 6 of 8 cases of psoriasis, the positive control (FIG. 2). CCL21 expression in the submucosal venule endothelium was also recognized by C8 in 2 of 3 cases of Crohn's disease (FIG. 5A-B), in 4 of 5 cases of ulcerative colitis (FIG. 5C-D), and in 4 of 6 cases of celiac sprue (FIG. 5E-F). No expression of CCL21 was observed in any of the 6 cases of synovium in rheumatoid arthritis (FIG. 2).

Discussion

CCL21 expression was consistently found to be induced in the endothelium of T-cell infiltrative autoimmune diseases of the skin, such as psoriasis (Christopherson K W 2nd, et al. Blood. 2003 Feb. 1; 101(3):801-6). Most lymphocytes present in those autoimmune skin samples were CCR7+. Because CCL21 is a potent and specific T-cell chemokine, this raised the intriguing question of whether its endothelial induction mediated the aberrant T-cell infiltration of the skin in those diseases. As described herein, it was tested whether venule endothelium in IBD expressed CCL21, and whether a monoclonal antibody could be generated that could target this CCL21 expression in IBD. Such an antibody could be then be developed as a biological therapy in IBD.

The results described herein demonstrated that the CCL21-mediated migration of multiple types of naïve helper T-cells was fully inhibited by the clone #8 monoclonal antibody. The expression of CCL21 in the colonic endothelium raises the possibility that this expression could mediate the migration of naïve T-cells to the submucosa for local lymphoid auto-antigen presentation. Indeed, CCL21 also promotes migration of naïve dendritic cells as well (12,13). Thus, expression of CCL21 by submucosal venule endothelium could be an important target for disruption of the biology of IBD (Sakai Y, Kobayashi M. Pathol Int. 2015 July; 65 (7):344-54; Wang L, et al. J Intern Med. 2015 October; 278(4):369-95; Singh U P, et al. Cytokine. 2016 January; 77: 44-9; Yao D, et al. Inflamm Bowel Dis. 2019 Jul. 9. pii: izz149; and Danese S, Fiocchi C. Dig Dis. 2016; 34(1-2):43-50).

In general, as naïve CD4⁺ T-cells mature to antigen-responding cells, they lose expression of CCR7, CD45RA and CD27 (Ruth D. Fritsch, et al. J Immunol Nov. 15, 2005, 175 (10) 6489-6497) and the cell's ability to migrate towards CCL21; unsurprisingly, as CCR7 is the main chemokine receptor for CCL21. Th cells without much expression of CCL21 still had some migration to CCL21 and that migration was partially blocked by clone #8 (FIG. 4). This is likely due to the ability of CCL21 to use the atypical T-cell chemokine receptor CRL1/ACKR4 to promote migration as well as CCR7, although to a much lesser extent (Ulvmar M H, et al. TNat Immunol. 2014 July; 15(7):623-30).

There is still a high fraction of IBD patients that suffer multiple intermittent relapses despite high compliance to therapy. In addition, steroid-resistant IBD has few options for effective therapy. Thus, new therapies for high-risk IBD would be beneficial. It was tested whether a monoclonal antibody could be generated that blocked CCL21-mediated T-cell migration. This would suggest that such an avenue for therapy of IBD could be effective (Yao D, et al. Inflamm Bowel Dis. 2019 Jul. 9. pii: izz149; Zhang Y Z, Li Y Y. World J Gastroenterol. 2014 Jan. 7; 20(1):91-9; Zhang H, et al. Genet Mol Res. 2014 Apr. 29; 13(2):3337-45; and Danese S, Fiocchi C. Dig Dis. 2016; 34(1-2):43-50). In this study, a monoclonal antibody that blocked the migration of naïve T-cells towards CCL21 was identified and that CCL21 expression was found on the endothelium of inflamed gut venules. It is noted that murine CCL21 is fairly divergent from human CCL21, and C8 does not bind well with murine CCL21 (FIG. 1).

The timing of therapy with C8 might be important to be effective. It is possible that an CCL21 antibody might decrease relapse of IBD after remission, but might not treat active disease, since by that time it may be too late to prevent recruitment of naïve T-cells for antigen presentation because such T-cells would already be activated. Thus, these results suggest that CCL21 is important for recruitment of naïve T-cells (and dendritic cells) to sites of mucosal antigen presentation in IBD. Further, other gut inflammatory diseases such as graft versus host disease (GVHD) may also be amenable to therapy with CCL21 blockade.

The data described herein provides evidence that autoimmune disease of the gut may be distinct from other autoimmune diseases (Bryant V L, Slade C A. Immunol Cell Biol. 2015 April; 93(4):364-7; Kolaczkowska E, Kubes P. Nat Rev Immunol. 2013 March; 13(3):159-75; Sakai Y, Kobayashi M. Pathol Int. 2015 July; 65 (7):344-54; Wang L, et al. J Intern Med. 2015 October; 278(4):369-95; and Singh U P, et al. Cytokine. 2016 January; 77: 44-9). Gut autoimmune disease may result more from local auto-antigen presentation given that the gut Payer's patches are secondary lymphoid organs. Draining lymph nodes are less likely to be the site of activation of naïve lymphocytes in gut autoimmune disease compared to other target organ autoimmune disease (Wang L, et al. J Intern Med. 2015 October; 278(4):369-95; and Singh U P, et al. Cytokine. 2016 January; 77: 44-9; Yao D, et al. Inflamm Bowel Dis. 2019 Jul. 9. pii: izz149; Zhang Y Z, Li Y Y. World J Gastroenterol. 2014 Jan. 7; 20(1):91-9; Zhang H, et al. Genet Mol Res. 2014 Apr. 29; 13(2):3337-45; and Danese S, Fiocchi C. Endothelial Cell-Immune Cell Interaction in IBD. Dig Dis. 2016; 34(1-2):43-50). Thus, local expression of venule endothelium CCL21 might be important for activation of naive lymphocytes in IBD (Zhang Y Z, Li Y Y. World J Gastroenterol. 2014 Jan. 7; 20(1):91-9; Zhang H, et al. Genet Mol Res. 2014 Apr. 29; 13(2):3337-45; and Danese S, Fiocchi C. Dig Dis. 2016; 34(1-2):43-50). Taken together, aberrant microbiome stimulation of local tissue inflammatory cytokines could activate abnormal CCL21 endothelial expression, which may arrest rolling naïve lymphocytes, and induce their diapedesis, where they can be presented with auto-antigen (Hromas R, et al. J Immunol. 1997; 159:2554-2558; Nagira M, et al. J Biol Chem. 1997; 272:19518-19524; Comerford I, et al. Cytokine Growth Factor Rev. 2013 June; 24(3):269-83; Förster R, et al. Nat Rev Immunol. 2008 May; 8(5):362-71; Gunn M D, et al. Proc Natl Acad Sci USA. 1998; 95:258-263; and Danese S, Fiocchi C. Dig Dis. 2016; 34(1-2):43-50). The basic carboxy terminus of CCL21 interacts with endothelial heparinoids and hyaluronic acids, maintaining it in position, and presenting the amino terminus for venule lymphocyte CCR7 binding (Nagira M, et al. J Biol Chem. 1997; 272:19518-19524; Comerford I, et al. Cytokine Growth Factor Rev. 2013 June; 24(3):269-83; Förster R, et al. CCR7 and its ligands: balancing immunity and tolerance. Nat Rev Immunol. 2008 May; 8(5):362-71; and Gunn M D, et al. Proc Natl Acad Sci USA. 1998; 95:258-263). Blocking naïve T-cell transmigration from the blood to the gut submucosa may lead to a decrease in the pathologic damage of these tissues by cytotoxic T cells. The results described herein provides evidence that by blocking CCL21 function with a monoclonal antibody described herein, may be therapeutic strategy to treat IBD.

### Example 2: A Humanized Monoclonal Antibody Against the Endothelial Chemokine CCL21 for the Diagnosis and Treatment of Inflammatory Bowel Disease

Chemokines are small proteins that promote leukocyte migration during development, infection, and inflammation. The isolated chemokine CCL21, a potent chemo-attractant for naïve T-cells, naïve B-cells, and immature dendritic cells. CCL21, has a 37 amino acid carboxy terminal extension that is distinct from the rest of the chemokine family, which is thought to anchor it to venule endothelium where the amino terminus can interact with its cognate receptor, CCR7. Venule endothelium expressing CCL21 plays an important role in attracting naïve immune cells to sites of antigen presentation. A series of monoclonal antibodies to the amino terminus of CCL21 were generated that blocked the interaction of CCL21 with its receptor CCR7. A humanized clone was identified that blocked naïve T-cell migration towards CCL21, while memory effector T-cells were less affected. Using this monoclonal antibody, the results demonstrated that CCL21 is expressed in the mucosal venule endothelium of the large majority of inflammatory bowel diseases (IBD), including Crohn's disease, ulcerative colitis, and also in celiac disease. This expression correlated with active IBD in 5 of 6 cases, whereas none of 6 normal bowel biopsies had CCL21 expression. This study raises the possibility that this monoclonal antibody could be used to diagnose initial or recurrent of IBD. Significantly, this antibody could also be used for therapeutic intervention in IBD by selectively interfering with recruitment of naïve immune effector cells to sites of antigen presentation, without harming overall memory immunity.

A series of anti-CCL21 monoclonal antibodies to the amino terminus of human CCL21 (Love M, et al. Biochemistry. 2012; 51(3):733-5) were generated and a clone that completely blocked naïve T-cell migration towards CCL21 was identified. However, migration of memory T-cell towards CCL21 was less affected. Using this monoclonal antibody (C8), the results demonstrated that CCL21 is expressed in the mucosal venule endothelium of the majority of inflammatory bowel diseases, including Crohn's disease, ulcerative colitis, and celiac disease. This data show that this monoclonal antibody can serve as a diagnostic marker of active IBD, and also prevent initiation or recurrence of inflammatory bowel diseases by selectively interfering with recruitment of naïve immune effector cells to sites of antigen presentation, without harming overall memory immunity (Yao D, et al. Inflamm Bowel Dis. 2019; 25(10): 1595-1602; Zhang Y Z, Li Y Y. World J Gastroenterol. 2014; 20(1):91-9; and Zhang H, et al. Genet Mol Res. 2014; 13(2):3337-45; and Danese S, Fiocchi C. Dig Dis. 2016; 34(1-2):43-50).

Materials and methods. Monoclonal antibody generation. Two BALB/C mice (Jackson Labs, Bar Harbor, ME) were subcutaneously immunized with 2 mg each of human CCL21 recombinant full-length protein in complete Freund's adjuvant and boosted three times with the equivalent preparation before splenic harvest. Euthanasia was performed by CO₂ inhalation followed by cervical dislocation and splenocytes harvested in a sterile manner. One hundred ninety-six fused plasma cell clones were screened, and that by ELISA produced an antibody that reacted with human CCL21 for binding to the amino terminus of human CCL21 protein. Two peptides from the amino terminus of hCCL21 were tested to determine whether they interact with CCR7 and could compete off the antibody clone binding to CCL21 using slot blot. Full-length recombinant mouse CCL21 protein was purchased from R&D Systems. Full-length recombinant human CCL21 protein was purchased from Novus Biologicals, Centennial, CO, USA. CCL21 (8-20 aa) and CCL21 (43-56 aa) peptides were synthesized by New England Peptide (Gardner, MA, USA). When the best performing murine clone was identified, it was humanized via gene conversion using preassembled oligonucleotide mutagenesis (Adair J R, et al. Hum Antibodies Hybridomas. 1994; 5(1-2):41-7; and Carter P, et al. Proc Nat Acad Sci May 1992; 89(10): 4285-4289). Sixteen human IgG1-LALA clones were generated that had varied characteristics for solubility, isomerization, glycosylation, free cysteines, or deamidation. None could fix complement.

Human peripheral blood T-cell isolation. Normal donor human peripheral blood (PB) was collected. Volunteer donors were recruited by public notice, signed an informed consent, and donated blood under sterile precautions. Mononuclear cell layer was collected using Ficoll-Paque PLUS (GE Healthcare Bio-Sciences AB; Pittsburgh, PA) density gradient centrifugation. The CD3+ PB cells were then isolated using immunoaffinity selection with MiniMACS paramagnetic CD3 microbeads (Miltenyi Biotec; Auburn, CA) using two sequential LS columns (Miltenyi Biotec, Auburn, CA).

T-cell chemotaxis assays. T-cell chemotaxis was measured (Hromas R, et al. J Immunol. 1997; 159:2554-2558; and Fritsch R D, et al. J Immunol 2005; 175 (10) 6489-6497). Human PB T-cells acclimated to 37° C. were suspended in prewarmed RPMI (37° C.) with bovine serum albumin (BSA; Sigma-Aldrich; St. Louis, MO). Costar 24-well transwell plates with 6.5 mm diameter inserts with 5.0 μm pores (Sigma-Aldrich, St. Louis, MO) were prepared by placing 650111 of prewarmed RPMI with 0.5% BSA that contained 0, 1200 ng/mL rhCCL21 (R&D Systems, Minneapolis, MN) or 1200 ng/mL rhCCL21 pretreated for 1 hour with sample clones #7-39 in the bottom well and allowing plates to acclimate at 37° C. for half an hour prior to chemotaxis assay. Cells were suspended at 300,000 cells/100 μl prewarmed RPMI with 0.5% BSA and loaded to the top chamber of the transwell assay. Transwell plates were placed in a 37° C. incubator (95% humidity, 5% CO2) for 4 hours. Percent migration was determined using flow cytometry (counts determined by running samples for the same amount of time at the same speed) with background migration (cells that migrated toward media alone; always <4%) subtracted from total migrated cells.

Flow cytometry. To analyze which T cell populations migrated in the chemotaxis assays (Fritsch R D, et al. J Immunol 2005; 175 (10) 6489-6497), after counting by flow, cells were washed in PBS, incubated in fluorescently conjugated anti-human antibody cocktail for minutes at room temperature, washed in PBS, and then fixed in 1% formaldehyde. The samples were analyzed on an LSR II flow cytometer (BD Biosciences; San Jose, CA). Antibody concentrations were used as stated by manufacturer's instructions. Data analysis was performed using FlowJo 7.6.3 software (Tree Star; Ashland, OR). Gates were determined using fluorescence minus one control. The following markers were used: APC-H7 conjugated anti-human CD3 (clone SK7), PerCP-Cy5.5 conjugated anti-human CD4 (clone SK3), FITC conjugated anti-human CD45RA (clone HI100), Alexa Fluor® 647 conjugated anti-human CD197 (CCR7; clone 150503) (components of a Human Naïve/Memory T cell Panel Kit from BD Biosciences cat. #561438; San Jose, CA, USA), BV421 conjugated anti-human CD8 (clone RPA-T8; BD Biosciences, San Jose, CA), PE conjugated anti-human CD27 (clone M-T271; BD Biosciences; San Jose, CA). For all antibodies used in these studies, the validation for the relevant species and applications can be found on the indicated manufacturer's website.

Immunohistology. Immunohistology was performed (Christopherson K W 2nd, et al. Blood. 2003; 101(3):801-6). The University of Mississippi Medical Center anatomic pathology database was searched for cases of psoriasis (positive controls), inflammatory bowel disease (Crohn's disease and ulcerative colitis), celiac sprue, and rheumatoid arthritis (negative controls). Samples were anonymized before sending for immunohistology. Hematoxylin and eosin-stained slides were reviewed to confirm diagnoses in order to select blocks for immunohistochemical staining. For each case, 4-micron thick sections were cut from the paraffin-embedded formalin-fixed tissue blocks and placed on charged slides. Sections were deparaffinized in xylene and rehydrated through graded alcohols to distilled water before undergoing antigen retrieval. Immunohistochemistry was performed using the Discovery Ultra automated instrument (Roche; Indianapolis, IN, USA) per the manufacturer's instructions, with the CCL21 antibody clone 8 at a 1:1000 dilution, and anti-mouse conjugated with DAB. Positive controls of normal human lymph node and negative controls of like tissue with mouse IgG were also performed. Immunoreactivity was qualitatively assessed by 2 pathologists. Counter-staining was performed with hematoxylin and eosin. Reactions were judged negative if there was no capillary endothelial expression, rare capillaries expressing, or weak or blush discontinuous expression. Staining was read blind to diagnoses, and classified as positive if there was multifocal, strong, and nearly circumferential capillary endothelial expression.

Results and Discussion. Generation of anti-CCL21 monoclonal antibodies against the amino terminus of human CCL21. The amino terminus of CCL21 is the region that interacts with its receptor, CCR7 (Love M, et al. Biochemistry. 2012; 51(3):733-5). BALB/C mice were subcutaneously immunized with human CCL21 recombinant full-length protein in adjuvant and boosted three times before splenic harvest. One hundred ninety-six fused plasma cell clones that by ELISA produced an antibody that reacted with human CCL21 for western blot recognition of human CCL21 protein as a single band were screened (FIG. 1A). Using slot blot analysis, titer was screened and the specificity of these antibodies was characterized (FIG. 1B). The amino terminus of CCL21 is the protein region that interacts with its receptor CCR7, while carboxy terminus is thought to interact with endothelial extracellular heparinoids, anchoring the chemokine to the endothelial cell surface (Gunn M D, et al. Proc Natl Acad Sci USA. 1998; 95:258-263; Christopherson K W 2nd, et al. Blood. 2003; 101(3):801-6; and Love M, et al. Biochemistry. 2012; 51(3):733-5). Screening was then performed by assessing whether peptides homologous to the amino terminus of CCL21 could compete off the monoclonal antibody in the slot blot analysis. Peptides containing amino acids 8-20 and 43-56 from the amino terminal CCR7-interacting regions of CCL21 were used to compete against the monoclonal antibody clones binding to CCL21 (FIG. 1C) (Love M, et al. Biochemistry. 2012; 51(3):733-5). High titer antibodies were also assessed by western analysis for specific binding to human CCL21 and not the related chemokine CCL19 (FIG. 1D). After these screens were complete, 33 clones were left for analysis that were selectively bound to the amino terminus CCL21 at a high titer. These clones were then assessed for inhibition of T-cell migration in chemotaxis assays.

Anti-CCL21 Monoclonal antibody blockade of human CCL21 function. Transwell chemotaxis assays were used to measure the ability of each of the 33 monoclonal antibody clones to block CD3⁺/CD4⁺ helper (h) T-cell migration from the top well to the lower well in response to a CCL21 gradient (FIG. 3). One clone (Clone #8, also referred to as “C8”) was identified that completely abrogated Th-cell chemotaxis towards CCL21 and one that partially blocked Th-cell chemotaxis towards CCL21 (Clone #9). No other clones had any significant inhibitory activity. Interestingly, clones #12-14, 25, 26, each enhanced chemotaxis towards CCL21 (FIG. 3A), implying that they improved presentation of CCL21 to its receptor CCR7 on Th-cells. The Th-cell chemotaxis inhibition assays were repeated with C8 on three total normal donors and the results show that C8 inhibited CCL21-directed migration for each of the three normal Th-cell samples, indicating that this was a general finding, and not individualized.

CCL21 promotes migration of naive helper T-cells but not memory T-cells. Using flow cytometric analysis of T-cell subsets, the type of T-cell that responds to CCL21 and were blocked by anti-CCL21 C8 was assessed. CD45RA and CD27 expression define naïve T-cells which have not yet been exposed to or responded to antigen (FIGS. 4A, B) (Fritsch R D, et al. J Immunol 2005; 175 (10) 6489-6497). The results show that CD3⁺/CD4⁺/CD45RA⁺/CD27⁺ naïve Th-cells migrated best to CCL21 and that this migration was nearly completely blocked by C8 (FIG. 4C). However, Th-cells that do not express CD27, which are cells that may have already responded to antigen, migrate with significantly less frequency to CCL21, and C8 was less successful in inhibited migration (FIGS. 4D-F). Clone 8 (C8) completely abrogated T-cell migration towards CCL21, while clone 9 partially inhibited migration. Several clones promoted migration of T-cells, suggesting they enhanced CCL21 presentation to its receptor, CCR7. CCL21 has a minor non-canonical receptor CCRL1 that can be responsible for residual migration towards CCL21 in CCR7⁻ T-cells (FIGS. 4E, F) (Ulvmar M, et al. Nat Immunol 2014; 15, 623-630).

Immunohistologic analysis of the expression of CCL21 in the endothelium of intestinal autoimmune diseases. It was first tested whether C8 and another murine monoclonal antibody among the original cohort that failed to bind to human CCL21 bound to appropriate cells in a human tonsil lymph node (FIG. 2, left upper panel). It was found that C8 bound to appropriate cells in the lymph node-dendritic cells, endothelial cells and lymphocytes whereas the negative control monoclonal antibody did not (FIG. 2, left lower panel). It was also tested whether C8 bound specifically to the endothelium of active psoriasis as a positive control, since T-cell-infiltrative autoimmune diseases of the skin expressed CCL21 in the venule endothelium (Christopherson K W 2nd, et al. Blood. 2003; 101(3):801-6). There was CCL21 expression in the dermal venule endothelium recognized by C8 in 6 of 8 cases of psoriasis, the positive controls (FIG. 2, right upper panel) (Christopherson K W 2nd, et al. Blood. 2003; 101(3):801-6). Rheumatoid arthritis was used as a negative control since T-cell infiltration in this autoimmune disease is mediated by a distinct mechanism (Wang L, et al. J Intern Med. 2015; 278(4):369-95). C8 did not bind to the endothelium of any of the 6 cases of synovium in rheumatoid arthritis (FIG. 2, right lower panel).

Next, the binding of C8 in biopsy specimens of intestinal autoimmune diseases (Crohn's disease, ulcerative colitis, and celiac sprue disease) was examined using immunohistology (FIG. 5). There was CCL21 expression in the submucosal venule endothelium recognized by C8 in 2 of 3 cases of Crohn's disease (FIG. 5A, B), in 3 of 3 cases of ulcerative colitis (FIG. 5C, D), and in 4 of 6 cases of sprue (FIG. 5E, F). There was no expression of CCL21 in 3 normal duodenal biopsies (FIG. 5G) or in 3 normal colonic biopsy specimens (FIG. 5H). Using chi square analysis for comparison of CCL21 endothelial expression in the IBD samples with the normal bowel samples, the 5 positives in the 6 IBD samples have a p value of 0.049 for being different from the 0 of 6 positives in the normal bowel specimens.

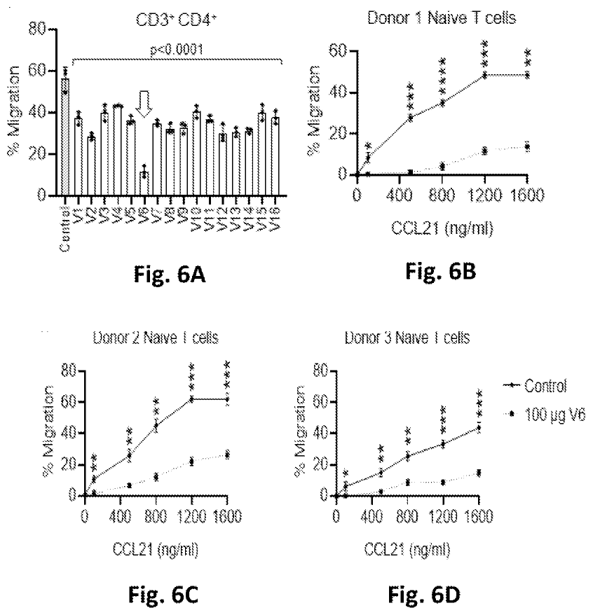

Humanization of C8 maintained inhibition of naïve T-cell migration towards CCL21. Sixteen humanized clones (V1 to V16) were generated from C8 (C8 comprises a light chain variable region amino acid sequence of SEQ ID NO: 7 and a heavy chain variable region amino acid sequence of SEQ ID NO: 14) and tested for inhibition of chemotaxis of helper T-cells by CCL21. While most of the clones decreased CD3⁺/CD4⁺ Th-cell chemotaxis, clone V6 was the most effective humanized clone at blocking helper T-cell migration towards CCL21 (FIG. 6). Clone V6 comprises a light chain variable region amino acid sequence of SEQ ID NO: 64 and a heavy chain variable region amino acid sequence of SEQ ID NO: 60. These data show that not all variations outside the CDRs during humanization were equal, and most harmed (e.g., decreased) the activity of C8 in blocking T-cell migration towards CCL21. Possible reasons for this observation are as follows: the structure of the human constant regions may have oriented the other humanized antibodies away from CCR7, in an opposite direction as C8; the murine constant regions may have had distinct glycosylation patterns compared to the humanized clones which may be important for interference with CCR7 interaction; aggregation of C8 may have been important for its interference between CCL21 and CCR7; and enhanced solubility was selected for the humanized clones. The humanized clones were each highly soluble, (88.7-99.3%, with V6 having 90.3% existing as a monomer in solution). This is advantageous for clinical antibody production but may have interfered with function. Additionally, the humanized clones, except for V6, may have motifs for deamidation and isomerization. Interestingly, there is one amino acid change between V6 (Thr) (SEQ ID NO: 64) and V7 (Ser) (SEQ ID NO: 65) in the VL region, but this site is facing externally and could potentially block interaction with CCR7 due to the steric hindrance of the added methyl group on Thr.

In sum, these data demonstrate that the murine-derived monoclonal antibody C8 can be humanized without loss of activity, improving its capability as a therapeutic agent.

In psoriasis, for example, it was found that CCL21 expression was consistently induced in the endothelium of T-cell infiltrative autoimmune diseases of the skin (Christopherson K W 2nd, et al. Blood. 2003; 101(3):801-6). Most lymphocytes present in those autoimmune skin samples were CCR7⁺ (Christopherson K W 2nd, et al. Blood. 2003; 101(3):801-6). Because CCL21 is a potent and specific naïve T-cell and dendritic cell chemokine, these data showed that CCL21's endothelial induction mediated the aberrant T-cell infiltration of the skin in those diseases. In this study, the results demonstrated that the CCL21-mediated migration of multiple types of naïve helper T-cells was fully inhibited by the C8 monoclonal antibody and its humanized counterpart V6. In general, as naïve CD4⁺ T-cells mature to antigen-responding cells, they lose expression of CCR7, CD45RA and CD27 (Fritsch R D, et al. J Immunol 2005; 175 (10) 6489-6497) and the cell's ability to migrate towards CCL21 decreases. Any residual chemotactic activity towards CCL21 after loss of CCR7 can be due to the minor receptor for CCL21, CCRL1 (Ulvmar M, et al. Nat Immunol 2014; 15, 623-630).

The expression of CCL21 in the inflamed intestinal endothelium raises two important clinically relevant points. First, the anti-CCL21 monoclonal antibody C8 recognized CCL21 venule endothelial expression in active IBD. Thus, C8 can serve as a diagnostic tool for early activation of IBD, before major tissue destruction or serve as a prognostic tool for severity of IBD, since IBD symptoms often do not mirror tissue biopsy histology.

The second clinically relevant point is the possibility that CCL21 expression in IBD could mediate the migration of naïve T-cells to the submucosa for local lymphoid auto-antigen presentation. Indeed, CCL21 also promotes migration of naïve dendritic cells as well, which could mature to locally present antigen to the incoming Th-cells (Comerford I, et al. Cytokine Growth Factor Rev. 2013; 24(3):269-83; and Förster R, et al. Nat Rev Immunol. 2008; 8(5):362-71). Thus, expression of CCL21 by submucosal venule endothelium may be an important target for disruption of the biology of IBD (Sakai Y, Kobayashi M. Pathol Int. 2015; 65(7):344-54; Wang L, et al. J Intern Med. 2015; 278(4):369-95; Singh U P, et al. Cytokine. 2016; 77:44-9; Yao D, et al. Inflamm Bowel Dis. 2019; 25(10): 1595-1602; Zhang Y Z, Li Y Y. World J Gastroenterol. 2014; 20(1):91-9; and Zhang H, et al. Genet Mol Res. 2014; 13(2):3337-45). The humanized anti-CCL21 monoclonal V6 antibody can thus serve as a biologic treatment to prevent the migration of T-cells and dendritic cells that ultimately would lead to mucosal destruction (Yao D, et al. Inflamm Bowel Dis. 2019; 25(10): 1595-1602; Zhang Y Z, Li Y Y. World J Gastroenterol. 2014; 20(1):91-9; and Zhang H, et al. Genet Mol Res. 2014; 13(2):3337-45).

It was also tested whether a monoclonal antibody could be generated that blocked CCL21-mediated T-cell migration. Such a finding would show that an avenue for therapy of IBD could be effective (Yao D, et al. Inflamm Bowel Dis. 2019; 25(10): 1595-1602; Zhang Y Z, Li Y Y. World J Gastroenterol. 2014; 20(1):91-9; and Zhang H, et al. Genet Mol Res. 2014; 13(2):3337-45). The results identified a monoclonal antibody that blocked the migration of naïve T-cells towards CCL21 and that identified expression of CCL21 on the endothelium of inflamed gut venules.

One strategy may be to administer a humanized anti-CCL21 monoclonal antibody (e.g., V6) to prevent relapse. In addition, administration of a humanized anti-CCL21 monoclonal antibody (e.g., V6) may also be useful in other gut inflammatory diseases such as graft versus host disease (GVHD).

The data described herein shows that autoimmune diseases of the gut may be distinct from other autoimmune diseases such as rheumatoid arthritis (Bryant V L, Slade C A. Immunol Cell Biol. 2015; 93(4):364-7; Kolaczkowska E, Kubes P. Nat Rev Immunol. 2013; 13(3):159-75; Sakai Y, Kobayashi M. Pathol Int. 2015; 65(7):344-54; Wang L, et al. J Intern Med. 2015; 278(4):369-95; and Singh U P, et al. Cytokine. 2016; 77:44-9). Gut autoimmune disease may be a result of local auto-antigen presentation given that the gut Payer's patches are secondary lymphoid organs. Draining lymph nodes are less likely to be the site of activation of naïve lymphocytes in gut autoimmune disease compared to other target organ autoimmune disease (Wang L, et al. J Intern Med. 2015; 278(4):369-95; Singh U P, et al. Cytokine. 2016; 77:44-9; Yao D, et al. Inflamm Bowel Dis. 2019; 25(10): 1595-1602; Zhang Y Z, Li Y Y. World J Gastroenterol. 2014; 20(1):91-9; Zhang H, et al. Genet Mol Res. 2014; 13(2):3337-45; and Danese S, Fiocchi C. Dig Dis. 2016; 34(1-2):43-50). Thus, local expression of venule endothelium CCL21 might play a role in activation of naive lymphocytes in IBD (Zhang Y Z, Li Y Y. World J Gastroenterol. 2014; 20(1):91-9; Zhang H, et al. Genet Mol Res. 2014; 13(2):3337-45; Danese S, Fiocchi C. Dig Dis. 2016; 34(1-2):43-50; and Fritsch R D, et al. J Immunol 2005; 175 (10) 6489-6497). For example, aberrant microbiome stimulation of local tissue inflammatory cytokines could activate abnormal CCL21 endothelial expression, which arrests rolling naïve Th-cells, and induces their diapedesis, where they can be presented with auto-antigen (Hromas R, et al. J Immunol. 1997; 159:2554-2558; Nagira M, et al. J Biol Chem. 1997; 272:19518-19524; Comerford I, et al. Cytokine Growth Factor Rev. 2013; 24(3):269-83; Förster R, et al. Nat Rev Immunol. 2008; 8(5):362-71; and Gunn M D, et al. Proc Natl Acad Sci USA. 1998; and Danese S, Fiocchi C. Dig Dis. 2016; 34(1-2):43-50). The basic, positive-charged carboxy terminus of CCL21 interacts with the negative-charged endothelial heparinoids and hyaluronic acids, maintaining CCL21 in position, and presenting its amino terminus for venule lymphocyte CCR7 binding (Nagira M, et al. J Biol Chem. 1997; 272:19518-19524; Comerford I, et al. Cytokine Growth Factor Rev. 2013; 24(3):269-83; Förster R, et al. Nat Rev Immunol. 2008; 8(5):362-71; Gunn M D, et al. Proc Natl Acad Sci USA. 1998; 95:258-263; and Love M, et al. Biochemistry. 2012; 51(3):733-5). Blocking naïve T-cell transmigration from the blood to the gut submucosa may lead to a decrease in the pathologic damage of these tissues by locally activated T-cells. And, as evidenced by the results described herein, blocking CCL21 endothelial function with any of the anti-CCL21 monoclonal antibodies disclosed herein can provide a therapeutic strategy in IBD.

Conclusion. Using a screening method, a murine monoclonal antibody against the human endothelial chemokine CCL21 that blocked naïve and partially activated T-cell chemotaxis towards CCL21 was identified. This monoclonal antibody identified expression of CCL21 in active inflammatory diseases of the bowel. However, there was no expression in normal bowel, thus, it may be useful as a diagnostic marker for active IBD. Multiple distinct humanized clones were generated using the hypervariable region of the C8 murine monoclonal antibody, and one of these, V6 was effective at blocking T-cell chemotaxis towards CCL21. Thus, this humanized monoclonal antibody against CCL21 (e.g., V6) can be an effective therapeutic agent for diagnosis of actively inflamed bowel and useful in preventing relapse of IBD.

1. An isolated antibody comprising a light chain variable region and a heavy chain variable region, wherein the light chain variable region comprises a complementarity determining region light chain 1 (CDRL1) amino acid sequence of SEQ ID NO: 1 or SEQ ID NO: 68; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10.

2. The isolated antibody of claim 1, comprising a light chain variable region amino acid sequence of SEQ ID NO: 7.

3. The isolated antibody of claim 1, comprising a heavy chain variable region amino acid sequence of SEQ ID NO: 14.

4. An isolated antibody comprising a light chain variable region amino acid sequence of SEQ ID NO: 7 and a heavy chain variable region amino acid sequence of SEQ ID NO: 14.

5. The isolated antibody of claim 2, wherein a light chain variable region has an amino acid sequence that is at least 90% identical to amino acid sequence of SEQ ID NO: 7.

6. The isolated antibody of claim 3, wherein a heavy chain variable region has an amino acid sequence that is at least 90% identical to amino acid sequence of SEQ ID NO: 14.

7. The isolated antibody of claim 1, wherein the antibody is recombinantly engineered, chimerized, or humanized.

8. The isolated antibody of claim 1, wherein the antibody is a Fab, an Fab′, an F(ab′)2, a Fv, a scFv, a diabody or fragments thereof.

9. The isolated antibody of claim 1, wherein the antibody binds to human CCL21 and does not cross-react with mouse CCL21.

10. The isolated antibody of claim 1, wherein the antibody binds to CCL21 with an affinity of greater than or equal to 42 nM.

11. The isolated antibody of claim 1, wherein the antibody selectively binds to human CCL21 and inhibits binding of human CCL21 to human CCR7.

12. An isolated antibody comprising a light chain variable region and a heavy chain variable region, wherein the light chain variable region comprises a complementarity determining region light chain 1 (CDRL1) amino acid sequence of SEQ ID NO: 1 or SEQ ID NO: 68; a determining region light chain 2 (CDRL2) amino acid sequence of SEQ ID NO: 2; and a determining region light chain 3 (CDRL3) amino acid sequence of SEQ ID NO: 3; and wherein the heavy chain variable region comprises a complementarity determining region heavy chain 1 (CDRH1) amino acid sequence of SEQ ID NO: 8; a complementarity determining region heavy chain 2 (CDRH2) amino acid sequence of SEQ ID NO: 9; and a complementarity determining region heavy chain 3 (CDRH3) amino acid sequence of SEQ ID NO: 10, wherein one or more of the CDRL1, CDRL2, CDRL3, CDRH1, CDRH2, or CDRH3 comprise 1, 2, 3, 4, or 5 conservative amino acid substitutions.

13. An isolated antibody comprising a light chain variable region amino acid sequence of SEQ ID NO: 7 and a heavy chain variable region amino acid sequence of SEQ ID NO: 14, wherein the isolated antibody comprises 1, 2, 3, 4, or 5 conservative amino acid substitutions in the light or heavy chain variable region amino acid sequences.

14. The isolated antibody of claim 1, wherein the antibody comprises a heavy chain variable region, wherein the heavy chain variable region comprises one or more complementarity determining region (CDRHs) CDRH1, CDRH2 and CDRH3 with amino acid sequences that have 0, 1, 2, 3, 4, or 5 conservative amino acid substitutions in 1, 2 or 3 CDRHs having the amino acid sequences of SEQ ID NO: 8, SEQ ID NO: 9, and SEQ ID NO: 10, respectively; and/or
wherein the antibody has a light chain variable region comprising one or more complementarity determining region (CDRLs) CDRL1, CDRL2 and CDRL3 with the amino acid sequences that have 0, 1, 2, 3, 4, or 5 conservative amino acid substitutions in 1, 2 or 3 CDRLs having the amino acid sequences of SEQ ID NO: 1 or SEQ ID NO: 68, SEQ ID NO: 2, and SEQ ID NO: 3, respectively.

15. The isolated antibody of claim 1, wherein the antibody specifically binds to an epitope of CCL21 comprising SEQ ID NO: 67.

16.-19. (canceled)

20. The isolated antibody of claim 1, wherein the antibody is linked to a detectable label.

21. (canceled)

22. The isolated antibody of claim 1, wherein the antibody is an IgG, an IgM, an IgA antibody or antigen binding fragment thereof.

23.-37. (canceled)

38. A composition comprising the isolated antibody of claim 1, and at least one pharmaceutically acceptable carrier or diluent.

39.-53. (canceled)

54. The isolated antibody of claim 1, comprising a light chain variable region amino acid sequence of SEQ ID NO: 63, SEQ ID NO: 64, SEQ ID NO: 65, or SEQ ID NO: 66.

55.-57. (canceled)

58. The isolated antibody of claim 1, comprising a heavy chain variable region amino acid sequence of SEQ ID NO: 59, SEQ ID NO: 60, SEQ ID NO: 61, or SEQ ID NO: 62.

59.-64. (canceled)


SEQUENCE LISTING (70 sequences)

SEQ ID NO: 1 - protein, 11 aa, Artificial Sequence
  Synthetic construct
HASQNINVWLS

SEQ ID NO: 2 - protein, 7 aa, Artificial Sequence
  Synthetic construct
KASNLHT

SEQ ID NO: 3 - protein, 9 aa, Artificial Sequence
  Synthetic construct
HQGHSFPLT

SEQ ID NO: 4 - protein, 6 aa, Artificial Sequence
  Synthetic construct
QNINVW

SEQ ID NO: 5 - protein, 3 aa, Artificial Sequence
  Synthetic construct
KAS

SEQ ID NO: 6 - protein, 9 aa, Artificial Sequence
  Synthetic construct
HQGHSFPLT

SEQ ID NO: 7 - protein, 107 aa, Artificial Sequence
  Synthetic construct
DIQMNQSPSSLSASLGDTVTITCHASQNINVWLSWYQQKPGNIPKLLIYKASNLHTGVPSRFSGSGSGTGFTLTISSLHPEDIATYYCHQGHSFPLTFGAGTKLELK

SEQ ID NO: 8 - protein, 5 aa, Artificial Sequence
  Synthetic construct
DFYLN

SEQ ID NO: 9 - protein, 17 aa, Artificial Sequence
  Synthetic construct
WIDPENGDTEYAPKFQG

SEQ ID NO: 10 - protein, 5 aa, Artificial Sequence
  Synthetic construct
FDYDY

SEQ ID NO: 11 - protein, 8 aa, Artificial Sequence
  Synthetic construct
GFNIKDFY

SEQ ID NO: 12 - protein, 8 aa, Artificial Sequence
  Synthetic construct
IDPENGDT

SEQ ID NO: 13 - protein, 7 aa, Artificial Sequence
  Synthetic construct
NAFDYDY

SEQ ID NO: 14 - protein, 114 aa, Artificial Sequence
  Synthetic construct
EVQLQQSGAEIVRSGASVRLSCTASGFNIKDFYLNWVKQRPEQGLDWIGWIDPENGDTEYAPKFQGKATMTADTSSNTIYLHLSSLTSEDTAVYYCNAFDYDYWGQGTTLTVSS

SEQ ID NO: 15 - protein, 114 aa, Artificial Sequence; identical to SEQ ID NO: 14
  Synthetic construct

SEQ ID NO: 16 - protein, 5 aa, Artificial Sequence
  Synthetic construct
EFYLN

SEQ ID NO: 17 - protein, 5 aa, Artificial Sequence
  Synthetic construct
DFYIN

SEQ ID NO: 18 - protein, 5 aa, Artificial Sequence
  Synthetic construct
EFYIN

SEQ ID NO: 19 - protein, 5 aa, Artificial Sequence
  Synthetic construct
DFYLQ

SEQ ID NO: 20 - protein, 5 aa, Artificial Sequence
  Synthetic construct
EFYLQ

SEQ ID NO: 21 - protein, 5 aa, Artificial Sequence
  Synthetic construct
EFYIQ

SEQ ID NO: 22 - protein, 17 aa, Artificial Sequence
  Synthetic construct
WIEPENGDTEYAPKFQG

SEQ ID NO: 23 - protein, 17 aa, Artificial Sequence
  Synthetic construct
WIDPDNGDTEYAPKFQG

SEQ ID NO: 24 - protein, 17 aa, Artificial Sequence
  Synthetic construct
WLDPENGDTEYAPKFQG

SEQ ID NO: 25 - protein, 17 aa, Artificial Sequence
  Synthetic construct
WIDPEQGDTEYAPKFQG

SEQ ID NO: 26 - protein, 17 aa, Artificial Sequence
  Synthetic construct
WIDPENGDTDYAPKFQG

SEQ ID NO: 27 - protein, 17 aa, Artificial Sequence
  Synthetic construct
WEDPENGDTEYAPKFNG

SEQ ID NO: 28 - protein, 17 aa, Artificial Sequence
  Synthetic construct
WIDPENGDTEYAPRFQG

SEQ ID NO: 29 - protein, 17 aa, Artificial Sequence
  Synthetic construct
WIDPEQGDTEYAPKFQG

SEQ ID NO: 30 - protein, 17 aa, Artificial Sequence
  Synthetic construct
WIEPDNGDTDYAPKFQG

SEQ ID NO: 31 - protein, 5 aa, Artificial Sequence
  Synthetic construct
FEYDY

SEQ ID NO: 32 - protein, 5 aa, Artificial Sequence
  Synthetic construct
FDYEY

SEQ ID NO: 33 - protein, 5 aa, Artificial Sequence
  Synthetic construct
FDFDY

SEQ ID NO: 34 - protein, 5 aa, Artificial Sequence
  Synthetic construct
FDYDF

SEQ ID NO: 35 - protein, 5 aa, Artificial Sequence
  Synthetic construct
YDYDY

SEQ ID NO: 36 - protein, 5 aa, Artificial Sequence
  Synthetic construct
FDFDY

SEQ ID NO: 37 - protein, 5 aa, Artificial Sequence
  Synthetic construct
FEYEY

SEQ ID NO: 38 - protein, 11 aa, Artificial Sequence
  Synthetic construct
HASQNIQVWLS

SEQ ID NO: 39 - protein, 11 aa, Artificial Sequence
  Synthetic construct
HASQNINVWIS

SEQ ID NO: 40 - protein, 11 aa, Artificial Sequence
  Synthetic construct
HASQNLNVWLS

SEQ ID NO: 41 - protein, 11 aa, Artificial Sequence
  Synthetic construct
HVSQNINLWLS

SEQ ID NO: 42 - protein, 11 aa, Artificial Sequence
  Synthetic construct
HASQNINVWLS

SEQ ID NO: 43 - protein, 11 aa, Artificial Sequence
  Synthetic construct
WASQNINVWLS

SEQ ID NO: 44 - protein, 7 aa, Artificial Sequence
  Synthetic construct
RASNLHT

SEQ ID NO: 45 - protein, 7 aa, Artificial Sequence
  Synthetic construct
KLSNLHT

SEQ ID NO: 46 - protein, 7 aa, Artificial Sequence
  Synthetic construct
KASNIHT

SEQ ID NO: 47 - protein, 7 aa, Artificial Sequence
  Synthetic construct
KASQLHT

SEQ ID NO: 48 - protein, 7 aa, Artificial Sequence
  Synthetic construct
KASNLWT

SEQ ID NO: 49 - protein, 7 aa, Artificial Sequence
  Synthetic construct
KASNLHS

SEQ ID NO: 50 - protein, 7 aa, Artificial Sequence
  Synthetic construct
KATNLHT

SEQ ID NO: 51 - protein, 7 aa, Artificial Sequence
  Synthetic construct
RASQLHT

SEQ ID NO: 52 - protein, 9 aa, Artificial Sequence
  Synthetic construct
HNGHSFPLT

SEQ ID NO: 53 - protein, 9 aa, Artificial Sequence
  Synthetic construct
HQGHTFPLT

SEQ ID NO: 54 - protein, 9 aa, Artificial Sequence
  Synthetic construct
HQGHSFPIT

SEQ ID NO: 55 - protein, 9 aa, Artificial Sequence
  Synthetic construct
HQGHSFPLS

SEQ ID NO: 56 - protein, 13 aa, Artificial Sequence
  Synthetic construct
SLLILVLAFGIPR

SEQ ID NO: 57 - protein, 13 aa, Artificial Sequence
  Synthetic construct
KYSQRKIPAKVVR

SEQ ID NO: 58 - protein, 107 aa, Artificial Sequence; identical to SEQ ID NO: 7
  Synthetic construct

SEQ ID NO: 59 - protein, 114 aa, Artificial Sequence
  Synthetic construct
EVQLVQSGAEVKKPGASVKVSCKASGFNIKDFYLNWVRQAPGQGLEWIGWIDPENGDTEYAPKFQGRVTMTADTSSNTIYMELSSLRSEDTAVYYCNAFDYDYWGQGTTVTVSS

SEQ ID NO: 60 - protein, 114 aa, Artificial Sequence
  Synthetic construct
QVQLVQSGAEVKKPGASVKVSCKASGFNIKDFYLNWVRQAPGQGLEWMGWIDPENGDTEYAPKFQGRVTMTADTSTNTIYMELSSLRSEDTAVYYCNAFDYDYWGQGTTVTVSS

SEQ ID NO: 61 - protein, 114 aa, Artificial Sequence
  Synthetic construct
EVQLVQSGSELKKPGASVKVSCKASGFNIKDFYLNWVRQAPGQGLEWIGWIDPENGDTEYAPGFTGRFVMSADTSSNTIYLQISSLKAEDTAVYYCNAFDYDYWGQGTTVTVSS

SEQ ID NO: 62 - protein, 114 aa, Artificial Sequence
  Synthetic construct
QVQLVQSGSELKKPGASVKVSCKASGFNIKDFYLNWVRQAPGQGLEWMGWIDPENGDTEYAPGFTGRFVFSADTSVNTIYLQISSLKAEDTAVYYCNAFDYDYWGQGTTVTVSS

SEQ ID NO: 63 - protein, 107 aa, Artificial Sequence
  Synthetic construct
DIQMTQSPSSLSASVGDRVTITCQASQNINVWLSWYQQKPGKAPKLLIYKASNLHTGVPSRFSGSGSGTDFTFTISSLQPEDIATYYCHQGHSFPLTFGGGTKLEIK

SEQ ID NO: 64 - protein, 107 aa, Artificial Sequence
  Synthetic construct
DIQMTQSPSSVSASVGDRVTITCRASQNINVWLSWYQQKPGKAPKLLIYKASNLHTGVPSRFSGSGSGTDFTLTISSLQPEDFATYYCHQGHSFPLTFGGGTKLEIK

SEQ ID NO: 65 - protein, 107 aa, Artificial Sequence
  Synthetic construct
DIQMTQSPSSVSASVGDRVTITCRASQNINVWLSWYQQKPGKAPKLLIYKASNLHSGVPSRFSGSGSGTDFTLTISSLQPEDFATYYCHQGHSFPLTFGGGTKLEIK

SEQ ID NO: 66 - protein, 107 aa, Artificial Sequence
  Synthetic construct
DIVMTQSPATLSLSPGERATLSCRASQNINVWLSWYQQKPGQAPRLLIYKASNLHTGIPARFSGSGSGTDFTLTISSLEPEDFAVYYCHQGHSFPLTFGGGTKLEIK

SEQ ID NO: 67 - protein, 13 aa, Artificial Sequence
  Synthetic construct
KYSQRKIPAKVVR

SEQ ID NO: 68 - protein, 11 aa, Artificial Sequence
  Synthetic construct
RASQNINVWLS

SEQ ID NO: 69 - protein, 11 aa, Artificial Sequence
  Synthetic construct
QASQNINVWLS

SEQ ID NO: 70 - protein, 17 aa, Artificial Sequence
  Synthetic construct
WIDPENGDTEYAPGFTG